SEQUENCE LISTING (85 sequences)

SEQ ID NO: 1 - RNA, 16 nt, artificial sequence
  D arm
gcgcaaucgguagcgc

SEQ ID NO: 2 - RNA, 38 nt, artificial sequence
  mitochondrial vector
gcgcaaucgguagcgccucuuuacagugcuuaguucuc

SEQ ID NO: 3 - DNA, 36 bp, artificial sequence
  mitochondiral vector
gcgcaaucgguagcgcgatgatgtggcctttggagt

SEQ ID NO: 4 - DNA, 36 bp, artificial sequence
  mitochondrial vector
gcgcaaucgguagcgcactccaaaggccacatcatc

SEQ ID NO: 5 - RNA, 18 nt, artificial sequence
  F-hairpin
gagcccccuacagggcuc

SEQ ID NO: 6 - RNA, 21 nt, artificial sequence
  example of nucleic acid for treating mitochondrial disease
ucuuuacagugcuuaguucuc

SEQ ID NO: 7 - DNA, 21 bp, artificial sequence
  example of nucleic acid for treating mitochondrial disease
tctttacagtgcttagttctc

SEQ ID NO: 8 - DNA, 12 bp, artificial sequence
  example of nucleic acid for treating mitochondrial disease
ttacagtgctta

SEQ ID NO: 9 - DNA, 13 bp, artificial sequence
  example of nucleic acid for treating mitochondrial disease
agtaagcactgta

SEQ ID NO: 10 - RNA, 16 nt, artificial sequence
  example of nucleic acid for treating mitochondrial disease
auguggccuuuggagu

SEQ ID NO: 11 - RNA, 20 nt, artificial sequence
  example of nucleic acid for treating mitochondrial disease
gaugauguggccuuuggagu

SEQ ID NO: 12 - RNA, 25 nt, artificial sequence
  example of nucleic acid for treating mitochondrial disease
augauguggccuuuggaguagaaac

SEQ ID NO: 13 - RNA, 16 nt, artificial sequence
  example of nucleic acid for treating mitochondrial disease
acuccaaaggccacau

SEQ ID NO: 14 - RNA, 20 nt, artificial sequence
  example of nucleic acid for treating mitochondrial disease
acuccaaaggccacaucauc

SEQ ID NO: 15 - RNA, 26 nt, artificial sequence
  example of nucleic acid for treating mitochondrial disease
guuucuacuccaaaggccacaucauc

SEQ ID NO: 16 - DNA, 20 bp, artificial sequence
  example of nucleic acid for treating mitochondrial disease
gatgatgtggcctttggagt

SEQ ID NO: 17 - DNA, 17 bp, artificial sequence
  example of nucleic acid for treating mitochondrial disease
actccaaaggccacatc

SEQ ID NO: 18 - DNA, 71 bp, artificial sequence
  Mitochondrial Targeting Sequence Insertion
gccacgcggttctttgacgcaagctgctgtaaggcttgttagggggtccctgcgcaaaacctcctgggcag

SEQ ID NO: 19 - DNA, 71 bp, artificial sequence
  Mitochondrial Targeting Sequence Insertion
ctgcccaggaggttttgcgcagggaccccctaacaagccttacagcagcttgcgtcaaagaaccgcgtggc

SEQ ID NO: 20 - DNA, 45 bp, artificial sequence
  Thrombin Cleavage Site and 66His Tag Deletion
ctttaagaaggagatatacatatgttgacgcaagctgctgtaagg

SEQ ID NO: 21 - DNA, 45 bp, artificial sequence
  Thrombin Cleavage Site and 66His Tag Deletion
ccttacagcagcttgcgtcaacatatgtatatctccttcttaaag

SEQ ID NO: 22 - DNA, 59 bp, artificial sequence
  His Tag Insertion at the C-terminus of preKARS2
caacagttggcagttctgtccaccatcaccatcaccattgagacccagctttcttgtac

SEQ ID NO: 23 - DNA, 59 bp, artificial sequence
  His Tag Insertion at the C-terminus of preKARS2
gtacaagaaagctgggtctcaatggtgatggtgatggtggacagaactgccaactgttg

SEQ ID NO: 24 - DNA, 75 bp, artificial sequence
  nucleotides amplified from yeast tRK1 T7-transcript
taatacgactcactatagcgcaatcggtagcgcctctttacagtgcttagttctcgagccccctacagggctctt

SEQ ID NO: 25 - DNA, 69 bp, artificial sequence
  nucleotides amplified from yeast tRK1 T7-transcript
taatacgactcactatagcgcaatcggtagcgcagtaagcactgtaaatgagccccctacagggctctt

SEQ ID NO: 26 - DNA, 55 bp, artificial sequence
  nucleotides amplified from yeast tRK1 T7-transcript
taatacgactcactatagcgcaatcggtagcgcctctttacagtgcttagttctc

SEQ ID NO: 27 - DNA, 60 bp, artificial sequence
  nucleotides amplified from yeast tRK1 T7-transcript
taatacgactcactataggtctttacagtgcttacttctcgagccccctacagggctcca

SEQ ID NO: 28 - RNA, 58 nt, artificial sequence
  nucleotides amplified from yeast tRK1 T7-transcript
gcgcaaucgguagcgccucuuuacagugcuuaguucucgagcccccuacagggcucuu

SEQ ID NO: 29 - RNA, 52 nt, artificial sequence
  nucleotides amplified from yeast tRK1 T7-transcript
gcgcaaucgguagcgcaguaagcacuguaaaugagcccccuacagggcucuu

SEQ ID NO: 30 - RNA, 38 nt, artificial sequence; identical to SEQ ID NO: 2
  nucleotides amplified from yeast tRK1 T7-transcript

SEQ ID NO: 31 - RNA, 43 nt, artificial sequence
  nucleotides amplified from yeast tRK1 T7-transcript
ggucuuuacagugcuuacuucucgagcccccuacagggcucca

SEQ ID NO: 32 - DNA, 21 bp, artificial sequence
  siRNA1
caacttgctcctttcacagcg

SEQ ID NO: 33 - DNA, 21 bp, artificial sequence
  siRNA2
aaggacaagtcattttctgat

SEQ ID NO: 34 - RNA, 43 nt, artificial sequence; identical to SEQ ID NO: 31
  mitichondrial vector

SEQ ID NO: 35 - DNA, 34 bp, artificial sequence
  anti-tRK1 probe
gagtcatacgcgctaccgattgcgccaacaaggc

SEQ ID NO: 36 - DNA, 15 bp, artificial sequence
  anti-HF RNA probe
tggagccctgtaggg

SEQ ID NO: 37 - DNA, 18 bp, artificial sequence
  anti-mt tRNAVal probe
gttgaaatctcctaagtg

SEQ ID NO: 38 - DNA, 20 bp, artificial sequence
  anti-cyt 5.8S rRNA probe
aagtgacgctcagacaggca

SEQ ID NO: 39 - DNA, 34 bp, artificial sequence; identical to SEQ ID NO: 35
  probe

SEQ ID NO: 40 - DNA, 20 bp, artificial sequence
  probe
ggccgcaagtgcgttcgaag

SEQ ID NO: 41 - DNA, 21 bp, artificial sequence
  probe
catccaagtactaccaggccc

SEQ ID NO: 42 - DNA, 17 bp, artificial sequence
  probe
tctccggtttacaagac

SEQ ID NO: 43 - DNA, 17 bp, artificial sequence
  probe
gctaagtaagcactgta

SEQ ID NO: 44 - DNA, 67 bp, artificial sequence
  primer
taatacgactcactatagcgcaatcggtagcgcactccaaaggccacatgagccccctacagggctc

SEQ ID NO: 45 - DNA, 67 bp, artificial sequence
  primer
taatacgactcactatagcgcaatcggtagcgcatgtggcctttggagtgagccccctacagggctc

SEQ ID NO: 46 - DNA, 71 bp, artificial sequence
  primer
taatacgactcactatagcgcaatcggtagcgcactccaaaggccacatcatcgagccccctacagggctc

SEQ ID NO: 47 - DNA, 71 bp, artificial sequence
  primer
taatacgactcactatagcgcaatcggtagcgcgatgatgtggcctttggagtgagccccctacagggctc

SEQ ID NO: 48 - DNA, 76 bp, artificial sequence
  primer
taatacgactcactatagcgcaatcggtagcgcgtttctactccaaaggccacatcatgagccccctacagggctc

SEQ ID NO: 49 - DNA, 76 bp, artificial sequence
  primer
taatacgactcactatagcgcaatcggtagcgcatgatgtggcctttggagtagaaacgagccccctacagggctc

SEQ ID NO: 50 - DNA, 25 bp, artificial sequence
  primer
gggatccataatacgactcactata

SEQ ID NO: 51 - DNA, 15 bp, artificial sequence
  primer
aagagccctgtaggg

SEQ ID NO: 52 - RNA, 52 nt, artificial sequence
  primer
gcgcaaucgguagcgcacuccaaaggccacaugagcccccuacagggcucuu

SEQ ID NO: 53 - RNA, 52 nt, artificial sequence
  primer
gcgcaaucgguagcgcauguggccuuuggagugagcccccuacagggcucuu

SEQ ID NO: 54 - RNA, 56 nt, artificial sequence
  primer
gcgcaaucgguagcgcacuccaaaggccacaucaucgagcccccuacagggcucuu

SEQ ID NO: 55 - RNA, 56 nt, artificial sequence
  primer
gcgcaaucgguagcgcgaugauguggccuuuggagugagcccccuacagggcucuu

SEQ ID NO: 56 - RNA, 61 nt, artificial sequence
  primer
gcgcaaucgguagcgcguuucuacuccaaaggccacaucaugagcccccuacagggcucuu

SEQ ID NO: 57 - RNA, 61 nt, artificial sequence
  primer
gcgcaaucgguagcgcaugauguggccuuuggaguagaaacgagcccccuacagggcucuu

SEQ ID NO: 58 - DNA, 19 bp, artificial sequence
  primer
gttcttgtagttgaaatac

SEQ ID NO: 59 - DNA, 22 bp, artificial sequence
  primer
catacctctcacttcaacctcc

SEQ ID NO: 60 - DNA, 18 bp, artificial sequence
  probe
gaacctctgactgtaaag

SEQ ID NO: 61 - DNA, 21 bp, artificial sequence
  probe
cgctacctctcttcctcgtgg

SEQ ID NO: 62 - DNA, 22 bp, artificial sequence
  primer
catacctctcacttcaacctcc

SEQ ID NO: 63 - DNA, 25 bp, artificial sequence
  primer
aggcgtttgtgtatgatatgtttgc

SEQ ID NO: 64 - DNA, 21 bp, artificial sequence
  primer
cgataaaccccgatcaacctc

SEQ ID NO: 65 - DNA, 23 bp, artificial sequence
  primer
ggttataatttttcatctttccc

SEQ ID NO: 66 - RNA, 76 nt, artificial sequence
  tRK1 D-arm and F-hairpin
gccuuguuggcgcaaucgguagcgcguaugacucuuaaucauaagguuagggguucgagcccccuacagggcucca

SEQ ID NO: 67 - RNA, 43 nt, artificial sequence
  hairpin Fig 5C
gggccgcacacacagcacacagacaccaacacacacggcccgc

SEQ ID NO: 68 - RNA, 38 nt, artificial sequence; identical to SEQ ID NO: 2
  synthetic

SEQ ID NO: 69 - DNA, 38 bp, artificial sequence
  synthetic
gcgcaaucgguagcgcctctttacagtgcttagttctc

SEQ ID NO: 70 - DNA, 29 bp, artificial sequence
  synthetic
gcgcaaucgguagcgcttacagtgcttac

SEQ ID NO: 71 - DNA, 29 bp, artificial sequence
  synthetic
gcgcaaucgguagcgcagtaagcactgta

SEQ ID NO: 72 - DNA, 33 bp, artificial sequence
  synthetic
ttacagtgcttacgagcccccuacagggcucuu

SEQ ID NO: 73 - DNA, 33 bp, artificial sequence
  synthetic
gtaagcactgtaagagcccccuacagggcucuu

SEQ ID NO: 74 - RNA, 39 nt, artificial sequence
  anti-replicative RNA
gcgcaaucgguagcgcuucgagcccccuacagggcucuu

SEQ ID NO: 75 - RNA, 20 nt, artificial sequence
  target sequence of anti-replicative RNA
ccuuuggaguagaaaccugu

SEQ ID NO: 76 - RNA, 20 nt, artificial sequence
  target sequence of anti-replicative RNA
gccuuuggaguagaaaccug

SEQ ID NO: 77 - RNA, 20 nt, artificial sequence
  target sequence of anti-replicative RNA
ggccuuuggaguagaaaccu

SEQ ID NO: 78 - RNA, 20 nt, artificial sequence
  target sequence of anti-replicative RNA
ugcgguuucgaugauguggc

SEQ ID NO: 79 - DNA, 39 bp, artificial sequence
  L-strand of target sequence of anti-replicative RNA
acgccaaagctactacaccggaaacctcatctttggaca

SEQ ID NO: 80 - DNA, 39 bp, artificial sequence
  H-strand of target sequence of anti-replicative RNA
tgcggtttcgatgatgtggcctttggagtagaaacctgt

SEQ ID NO: 81 - RNA, 56 nt, artificial sequence
  RNA complementary to mutated ND5 gene
gcgcaaucgguagcgcugcgguuucgaugauguggcgagcccccuacagggcucuu

SEQ ID NO: 82 - RNA, 56 nt, artificial sequence
  RNA complementary to mutated ND5 gene
gcgcaaucgguagcgcgguuucgaugauguggccuugagcccccuacagggcucuu

SEQ ID NO: 83 - RNA, 52 nt, artificial sequence; identical to SEQ ID NO: 29
  mitochondrial vector

SEQ ID NO: 84 - DNA, 16 bp, artificial sequence
  cholesterol carrier
gcgctaccgattgcgc

SEQ ID NO: 85 - RNA, 35 nt, artificial sequence
  mitochondrial vector
aguaagcacuguaaagagcccccuacagggcucca

(19) United States
(12) Patent Application Publication
(10) Pub. No.: US 2015/0361450 A1
(43) Pub. Date: Dec. 17, 2015

(54) VECTOR FOR THERAPY OF MITOCHONDRIAL DISEASE

(71) Applicants: UNIVERSITE DE STRASBOURG, Strasbourg (FR); CENTRE NATIONAL DE LA RECHERCHE SCIENTIFIQUE, Paris (FR)

(72) Inventors: IVAN TARASSOV, STRASBOURG (FR); NINA ENTELLIS, STRASBOURG (FR)

(21) Appl. No.: 14/739,043

(22) Filed: Jun. 15, 2015

Related U.S. Application Data
(60) Provisional application No. 62/011,755, filed on Jun. 13, 2014.

(51) Int. Cl. C12N 15/85

(52) U.S. Cl. CPC C12N 15/85

(57) ABSTRACT

The present invention relates to mitochondrial RNA vectors with which a nucleic acid of interest may be imported into human mitochondria, in particular to suppress negative effects of mutations in mtDNA by affecting the level of heteroplasmy.

### CROSS-REFERENCE TO RELATED APPLICATION

This application claims the benefit of U.S. Provisional Application Ser. No. 62/011,755, filed Jun. 13, 2014, the disclosure of which is hereby incorporated by reference in its entirety, including all figures, tables and amino acid or nucleic acid sequences.

The Sequence Listing for this application is labeled “Seq-List.txt” which was created on Jun. 15, 2015 and is 17 KB. The entire contents of the sequence listing is incorporated herein by reference in its entirety.

### FIELD OF THE INVENTION

Defects in mitochondrial genomes can cause a wide range of clinical disorders, mainly neuromuscular diseases. Up to now, no efficient therapeutic treatment has been developed against this class of pathologies. Since most of deleterious mitochondrial mutations are heteroplasmic, meaning that wild-type and mutated forms of mitochondrial DNA (mtDNA) coexist in the same cell, the same cell, the shift in proportion between mutant and wild-type molecules could restore mitochondrial functions. The present invention relates to a mitochondrial RNA vector with which a nucleic acid of interest may be imported into human mitochondria to suppress negative effects of mutations in mtDNA by affecting the level of heteroplasmy.

### BACKGROUND OF THE INVENTION

RNA is increasingly used in therapeutic applications, including the agents of RNA interference, catalytically active RNA molecules and RNA aptamers that bind proteins and other ligands (1). Involved in many essential cellular pathways ranging from cellular respiration to apoptosis, mitochondria are subcellular organelles essential for eukaryotic cells containing their proper genome, mtDNA. Human mtDNA is a closed circular double-stranded molecule of 16.5 kb able to replicate autonomously and encoding only 13 polypeptides, 2 ribosomal RNA (12S and 16S) and 22 tRNA, the vast majority of mitochondrial proteins and several RNAs being encoded in the nucleus and imported from the cytoplasm. Multiple alterations may occur in the mitochondrial genome (deletions, duplications, point mutations) resulting in severe impact on cellular respiration and therefore leading to many diseases, essentially muscular and neurodegenerative disorders. To date, more than 300 pathologic diseases were shown to be caused by defects in mtDNA (2). The majority of these mutations are heteroplasmic, meaning that mtDNA coexists in 2 forms, wild-type and mutated, in the same cell. The occurrence and severity of pathologic effects depend on the heteroplasmy level, clinical symptoms appearing at the threshold of the order of 60 to 80%, depending on the mutation and the type of cells (3). Various strategies have been proposed to address these pathologies, unfortunately for the vast majority of cases no efficient treatment is currently available. In some cases, defects may be rescued by targeting into mitochondria nuclear DNA-expressed counterparts of the affected molecules, an approach called allotopic strategy (4,5). Allotropic expression of mtDNA-encoded polypeptides has been demonstrated in yeast, but in mammalian mitochondria results are contradictory. Another version of allotropic approach has exploited RNA mitochondrial import pathway, which is the only known natural mechanism of nucleic acid delivery into mitochondria (6), to develop two successful models of allotopic rescue of a mtDNA mutation by targeting recombinant tRNA into mitochondria and partially rescue negative effects of mutations in either mitochondrial tRNA-Lys or tRNA-Leu, causing MERRF and MELAS diseases, respectively (7,8).

Alternative strategy that might be termed as “anti-genomic”, which consists in addressing into mitochondria endonucleases specifically eliminating mutated mtDNA, was also described (9,10). The approach described here, referred to as anti-replicative, aims to induce a shift in heteroplasmy level by targeting specifically the replication of mutant mtDNA, thus giving a propagative advantage to wild-type genomes. This strategy has first been tested in vitro using peptide nucleic acids (PNAs) with high affinity to mutant mtDNA (11). It was demonstrated that PNA oligomers complementary to mutant mtDNA can specifically inhibit its replication in vitro, however, it remained non-applicable to living cells due to impossibility to introduce PNA molecules into mitochondria in vivo (12).

To overcome this impossibility to introduce PNA molecules into mitochondria in vivo, it was proposed to use RNA mitochondrial import.

### SUMMARY OF THE INVENTION

The present invention provides short synthetic RNAs comprising just one of the two domains of yeast tRNA^{Lys}CUU (tRK1) alternative structure, D-arm or F-hairpin. These molecules, fused to oligonucleotide stretches complementary to the mtDNA mutated region, were able to shift a heteroplasmy level in cells containing a large deletion or point mutation mtDNA, providing the first validation of the anti-replicative approach in vivo.

Accordingly, the present invention relates to mitochondrial vector comprising or consisting essentially of a first nucleic acid sequence linked to a second nucleic acid sequence,

wherein the first nucleic acid sequence is the sequence of D-arm as disclosed in SEQ ID NO: 1 or a sequence having at least 75% sequence identity with SEQ ID NO: 1 or the sequence of F-hairpin as disclosed in SEQ ID NO: 5 or a sequence having at least 75% sequence identity with SEQ ID NO: 5;

wherein the second nucleic acid sequence is a sequence of interest; and

wherein the mitochondrial vector does not comprise both sequences SEQ ID NOs: 1 and 5 or sequences having at least 75% sequence identity with SEQ ID NOs: 1 and 5.

Preferably, the first nucleic acid sequence is a sequence having at least 90% sequence identity with SEQ ID NOs: 1 or 5. More preferably, the first nucleic acid sequence is a sequence having at least 95% sequence identity with SEQ ID NOs: 1 or 5. Preferably, the first nucleic acid sequence is made of ribonucleotides.

Preferably, the second nucleic acid sequence is a wild-type mitochondrial DNA sequence or an altered mitochondrial DNA sequence or a complementary sequence thereof. More preferably, the second nucleic acid sequence is an altered mitochondrial DNA sequence or a complementary sequence thereof. Optionally, the second nucleic acid sequence of interest is between 10 to 30 nucleotides in length. Optionally, the second nucleic acid sequence comprises deoxyribonucleotides, ribonucleotides or a mixture thereof, and/or 2′-O-methylated ribonucleotide(s) and/or 3′-3′ or 5′-5′ inverted nucleotide.

Preferably, the mitochondrial vector is between 26 to 48 nucleotides in length.

Preferably, the sequence of D-arm as disclosed in SEQ ID NO: 1 or a sequence having at least 75% sequence identity with SEQ ID NO: 1 is linked at the 5′ end of the second nucleic acid sequence. More preferably, the sequence of D-arm as disclosed in SEQ ID NO: 1 or a sequence having at least 75% sequence identity with SEQ ID NO: 1 is at the 5′ end of the mitochondrial vector.

Preferably, the sequence of F-hairpin as disclosed in SEQ ID NO: 5 or a sequence having at least 75% sequence identity with SEQ ID NO: 1 is linked at the 3′ end of the second nucleic acid sequence. More preferably, the sequence of F-hairpin as disclosed in SEQ ID NO: 5 or a sequence having at least 75% sequence identity with SEQ ID NO: 5 is at the 3′ end of the mitochondrial vector or is located before the 1-5 nucleotides of the 3′ end of the mitochondrial vector.

Optionally, the mitochondrial vector is conjugated with delivery system such as a cholesterol.

The present invention also relates to a pharmaceutical composition comprising a mitochondrial vector of the present invention and a pharmaceutically acceptable carrier or excipient.

The present invention further relates to a method of treating in a subject a mitochondrial disease caused by a mutation in a gene in the mitochondrial genome which comprises administering to the subject the mitochondrial vector of the present invention, wherein the second nucleic acid sequence is a sequence comprising the mutation of the gene or its complementary sequence. Preferably, the mutation in a gene in the mitochondrial genome is selected in the group consisting of: large deletions causing KSS (Kearns Sayre Syndrome) or Pearson's disease (common deletion), point mutations in tRNAleu-A3243G, A3251 G, A3303G, T3250C, T3271 C and T3394C; tRNAlys-A8344G, G11778A, G8363A, T8356C; ND1-G3460A; ND4-A10750G, G14459A; ND6-T14484A; 12S rRNA-A1555G; MTTS2-C12258A; ATPase 6-T8993G, T8993C; tRNASer(UCN)-T7511C; 11778 and 14484, LHON mutations as well as mutations or deletions in ND2, ND3, ND5, cytochrome b, cytochrome oxidase I-III, and ATPase 8.

Finally, the present invention relates to a kit comprising a mitochondrial vector according to the present invention.

### BRIEF DESCRIPTION OF THE FIGURES

The patent or application file contains at least one drawing executed in color. Copies of this patent or patent application publication, with color drawing(s), will be provided by the Office upon request and payment of the necessary fee.

FIGS. 1A-1C: Predicted structures of the yeast tRNA^{Lys}CUU (tRK1) and small synthetic RNAs. (A) Two alternative structures of tRK1, as in (14). The cloverleaf structure is shown at the left, the F-structure at the right. The tRK1 amino acceptor stem is in red, the D-arm in blue and the T-arm in purple. (B) Secondary structures of small synthetic “anti-replicative” RNAs composed of the tRK1 D-arm (in blue) and the F helix-loop structure (in red), separated by oligonucleotide stretches complementary to the heavy or light strands of human mitochondrial DNA (14). (C) Truncated RNA molecules derived from FD-L RNA lacking either the D-arm of tRK1 (HF RNA) or the F-hairpin (HD RNA). The nucleotides added to the 59-end of HF RNA to improve T7-transcription are underlined. For HF RNA, only one secondary structure (dG=211.6 kcal/mol) was predicted by Mfold, for HD RNA, a structure with the minimal initial dG=25.9 kcal/mol is shown.

FIGS. 2A-2D: Interaction of the purified KARS2 and preKARS2 proteins with RNAs tested by EMSA. After incubation of 32P-labeled tRK1 (A), FD-L and FD-H RNAs (B) or the truncated HD and HF RNAs (C) with increasing concentrations of the recombinant proteins (indicated above the panels, in nM), the complex formation was visualized by autoradiography. In each assay, the bottom band corresponds to the free RNA species, the RNA-protein complex is marked with an arrowhead. The deduced dissociation constants (Kd) for each RNA are given at the bottom of the panel (in nM). A representative of at least three independent experiments is shown for each RNA. (D) North-Western hybridization. Membrane stripes, containing equal amounts of preKARS2, were incubated with labeled RNAs (indicated above). Lane 1, no competitor added; lane 2, hybridization in the presence of 30× molar excess of nonspecific competitor (rRNA E. coli); lane 3, hybridization in the presence of 10× molar excess of nonlabeled RNA FD-R. Left panel represents the membrane stained with Ponceau Red.

FIGS. 3A-3C: Import of RNA into isolated HepG2 mitochondria. Autoradiographies of RNA isolated from purified mitochondria and separated in denaturing 10% PAAG are presented. (A) Import of yeast tRK1, +*, yeast enolase was added instead of rabbit one. (B) Import of the small synthetic RNAs FD-L and FD-H. (C) Import of the truncated HD and HF RNAs. The name and position of the full-size RNA is indicated by an arrow on the left of each panel. Input, 2-5% of the RNA used for each assay (as indicated above the lane), corresponding to 60-150 fmoles of labeled RNA. Mitochondria (+) corresponds to the complete import assay, Mitochondria (−) to the mock import assay without mitochondria used as a control for non-specific protein-RNA aggregation. The RNA import efficiency was calculated by comparing the signal with the input and is indicated below each lane. A representative of three independent experiments is presented for each RNA, ±SD indicated.

FIGS. 4A-4D: Implication of preKARS2 in the RNA mitochondrial import in vivo. (A) Western blot analysis of preKARS2 downregulation by RNA interference (Si). The level of preKARS2 in the cells transiently transfected with siRNAs against preKARS2 (Si) compared to the control cells transfected with a control siRNA (Ctrl) is indicated below the panel. The antibodies used for immunodecoration are shown on the right. (B) Northern blot hybridization of the total and purified mitochondrial (mtRNA) RNAs isolated from the control cells (Ctrl) and the cells transfected with siRNAs against preKARS2 (Si), in 32 h after transfection with tRK1, FD-L or FD-H RNA, as indicated. The hybridization probes are shown on the right. The mt tRNA^{Val} probe was used as loading control, and the cytosolic 5.8S rRNA probe was used to confirm the absence of cytosolic RNA contamination in the mitochondrial RNA preparations. The relative RNA import efficiencies, taken as 1 for the control cells, are shown below each panel (see Methods for the import efficiency calculation). For each RNA, the results of at least three independent experiments are shown at the lower panel, ±SD indicated. (C) Western blot analysis of preKARS2 overexpression (OE), the relative level of overexpression is indicated below the panel. Ctrl, control cells transfected with an empty vector. (D) Analysis of the in vivo import of tRK1 and the small synthetic FD-L and FD-H RNAs into mitochondria of the control cells (Ctrl) and the preKARS2-overexpressing (OE) cells in 48 h after transfection with the corresponding RNAs. All indications are as in B.

FIGS. 5A-5E: Import of small truncated RNAs into mitochondria. (A) In vivo import of the truncated HF (left panel) and HD (right panel) RNAs in mitochondria of the control cells and the cells overexpressing preKARS2. (B) In vivo import of HF and HD RNAs in the control cells and the cells with downregulation of preKARS2. Hybridization probes are indicated on the right of the panels. Overexpression and downregulation of preKARS2 were confirmed by Western blot as in FIG. 4A, C. The relative import efficiencies are shown on the right panels, ±SD calculated from three independent experiments. (C) Secondary structure (on the left) of the artificial control RNA, predicted by Mfold. The control RNA in vitro (middle panel, indications are as in FIG. 3) and in vivo (right panel) import tests. (D) OD₂₈₀ absorption profile of HepG2 proteins separated by gel filtration on a Sephacryl G-200 column. (E) Import of HF RNA (upper panel) and HD RNA (lower panel) into isolated HepG2 mitochondria in the presence of proteins from the gel filtration fractions indicated above the lanes. Input, 2-5% of RNA used for each assay. The RNA import efficiencies calculated by comparing with the inputs, in fmoles of imported RNA per 0.1 mg of mitochondrial protein, are given below each lane. On each panel, a representative of at least three independent experiments is shown, ±SD indicated.

FIG. 6: Structures and nucleotide modifications of synthetic oligonucleotides.

FIG. 7: Stability of synthetic oligonucleotides in transiently transfected cybrid cells. Northern hybridization of total RNA isolated in 2-6 days after cell transfection (“D2-6”, as indicated above the lanes) with various SO-specific ³²P-labelled probes (the SO is indicated above each panel, the probe—at the left). Probes used for hybridization: D-loop or KSSpart, specific for SO used for transfection; 5S, against 5S rRNA to quantify the level of SO in the cells. Below each panel, a half-life time for corresponding SO is indicated; ±SD was calculated from n=3 independent experiments.

FIG. 8: Mitochondrial import of synthetic oligonucleotides in transiently transfected cybrid cells. On the left panel, an example of Northern hybridization of RNA extracted from purified mitochondria 48 h after transfection. Above the autoradiographs, SO used for transfection are indicated. On the left, hybridization probes: D-loop, specific for SO used for the transfection; 5.8S, against 5.8S rRNA to check the absence of cytosolic contamination in mitochondrial samples; tRNA^{Thr}, against mitochondrial tRNA to normalize the level of recombinant RNA to amount of loaded material. On the right panel, the diagram shows relative efficiencies of mitochondrial import. Import efficiency of D22L RNA was taken as 1. In all series ±SD=0.1, resulting from n=3 independent experiments.

FIG. 9: Ability of synthetic oligonucleotides to discriminate wild type and mutant mtDNA. Southern hybridization of wild-type (WT) or mutant (KSS) mtDNA fragments with labelled SO (indicated above each panel) under physiological conditions. The Ethidium Bromide stained gel is presented in the upper panel.

FIG. 10: Effect of synthetic oligonucleotides on heteroplasmy level in transfected cybrid cells. Time dependence of KSS deletion heteroplasmy level followed during 6-8 days after transfection of cybrid cells with various SO, indicated above each graph. ±SD was calculated from at least three independent experiments; *, statistically significant data.

FIG. 11: Design of anti-replicative RNAs targeting A13514>G mutation in human mtDNA. Human mtDNA genetic map and sequence of the mutated region of ND5 gene are presented, A13514>G mutation shown in bold; FD-RNA stem-loop import determinants are in grey. Design of recombinant RNA FD20H containing 20-mer corresponding to the H-strand of mtDNA is shown, see text for details.

FIGS. 12A-12C: Ability of recombinant molecules to discriminate wild type and mutant mtDNA. (A) Predicted secondary structures of anti-replicative molecules. Stem-loop import determinants are shown in grey. D20H-DNA and D20L-DNA, chemically synthesized chimeric molecules, RNA insertion has been replaced by DNA one. (B) Southern hybridization of wild type (WT) or mutant (A13514G) mtDNA fragments with labelled recombinant RNAs (indicated at the left) under physiological conditions. Long exp., radiogram after longer exposition for FD16H and FD16L. The specific radioactivity of all the RNA probes were comparable. (C) Graphical representation of hybridization specificity of different recombinant molecules (indicated below the graphs). The hybridization specificity was calculated for each RNA as 1−(ratio between hybridization signals for wild type and mutated DNA fragments). Thus, for recombinant RNA annealed only to mutant DNA fragment, the hybridization specificity value reached 1, and for RNA annealed equally to mutant and wild-type fragments, this value was close to zero. ±SD calculated from 3 independent experiments.

FIGS. 13A-13B: Confocal microscopy of human cells transfected with fluorescently labelled RNA. (A) Confocal microscopy of 143B cells transfected with Alexa-Fluor 488 labelled FD20H RNA (green) at various time periods after transfection (as indicated above the panels). Control, cells transfected with Alexa Fluor 488-5-UTP labelled RNA, which is not imported into mitochondria, 3 days post transfection. TMRM, visualization of mitochondrial network by red staining Below the panels, quantification of RNA co-localization with mitochondria, estimated for multiple cells and 6-10 optical sections in two independent experiments. PC, Pearson's correlation coefficient; M1 and M2, Manders' overlap coefficients, representing the percentage of green fluorescence co-localized with the red one (for M1) and the percentage of red fluorescence co-localization with the green one (for M2). (B) Magnification of the image corresponding to Day 4 post transfection. A 3D reconstruction of the confocal microscopy image by ImageJ software is presented in Supplementary video files (two channels and merge).

FIGS. 14A-14B: Anti-replicative RNA stability and mitochondrial import in transiently transfected cybrid cells. (A) On the top, an example of Northern hybridization of total RNA isolated in 3-6 days after cell transfection (as indicated below) with various recombinant molecules (indicated above the panels). Probes used for hybridization: D-loop, specific for all recombinant molecules used for transfection; 5S, against 5S rRNA to quantify the level of recombinant RNA in the cells. On the bottom, time-dependence of RNA decay, ±SD calculated from at least 4 independent experiments. (B) Mitochondrial import of recombinant molecules in transiently transfected cybrid cells. Northern hybridizations of RNA extracted from cells (Total RNA) or purified mitoplasts (Mito RNA) 48 h after transfection. Above, RNAs used for transfection are indicated. On the left, hybridization probes: D-loop, specific for recombinant molecules used for the transfection; Cyt 5.8S, against 5.8S rRNA to check the absence of cytosolic RNA in mitochondrial samples; snRNA U3, demonstrating that the mitoplast fraction was not contaminated by the small nuclear RNAs; Mit tRNA^{Val}, to normalize the level of recombinant RNA to amount of loaded material. Import efficiency was estimated as a ratio of mitochondrial to total signal for the D-loop probe after normalization of both signals to those for the mitochondrial tRNA^{Val} probe.

FIGS. 15A-15C: The effect of recombinant RNA on heteroplasmy level in transfected cybrid cells. (A) Time dependence of A13514G mutation heteroplasmy level (axis Y) followed during 7-8 days. D0-D8 (axis X), days after transfection of cells with different recombinant molecules. Mut, mock-transfected cybrid A13514G cells; WT, mock-transfected 143B cells; FD20L, FD20H or FD20L-DNA, cybrid cells transfected with corresponding recombinant molecule; Fibro 20H, primary fibroblasts transfected with FD20H RNA. Data are expressed as mean±s.d. for 3-5 independent experiments. Unpaired t-test between values for Mut and transfected cells; *P<0.02, **P<0.003, ***P<0.0009. (B) An example of RFLP analysis for DNA samples isolated in 3-8 days (as indicated above) after cybrid cells transfection with recombinant molecules indicated at the left. Digestion control was performed for each reaction, as shown at the right panel. For this, 600-bp PCR fragment of mtDNA was cleaved by HaeIII to 350 and 250-bp fragments in the same tube as 125-bp PCR fragment spanning A13514G mutation site. The A13514G mutation creates a HaeIII-specific cleavage site, giving the fragment of 80 bp. Size of fragments is indicated for each panel. (C) Evaluation of total and energised mitochondria content in 143B (Wild-type) and cybrid cells (A13514G) transfected with RNA FD20H during 8 days after cell transfection. MitoTracker Green and TMRM fluorescence normalized to the number of cells, values of wild-type cells were taken as 1.

FIG. 16: Conjugate Cholesterol-mitochondrial vector with different sort of biodegradable linkers, and a fluorescent probe. Effect of molecules on heteroplasmic rate of mtDNA mutation (MELAS). Cells: Fibroblasts.

### DETAILED DESCRIPTION OF THE INVENTION

In some embodiments of the present disclosure, a mitochondrial vector delivers polynucleotides to mitochondria. Mitochondria contain the molecular machinery for the conversion of energy from the breakdown of glucose into adenosine triphosphate (ATP). The energy stored in the high-energy phosphate bonds of ATP is then available to power cellular functions. Mitochondria are constituted by >1000 proteins, many lipids forming a double membrane, DNA, RNA and many metabolites. These organelles have an outer membrane surrounding an inner membrane that folds into a scaffolding (cristae) for oxidative phosphorylation and electron transport enzymes. Most mitochondria have flat shelf-like cristae, but those in steroid secreting cells may have tubular cristae. The mitochondrial matrix contains the enzymes of the citric acid cycle, fatty acid oxidation and mitochondrial nucleic acids.

Mitochondrial DNA in humans is double stranded and circular. Mitochondrial RNA comes in three standard varieties: ribosomal, messenger and transfer, but each is specific to the mitochondria. Some protein synthesis occurs in the mitochondria on mitochondrial ribosomes that are different from cytoplasmic ribosomes. Other mitochondrial proteins are made on cytoplasmic ribosomes with a signal sequence (cleavable or non-cleavable N-terminal or non-cleavable internal) that directs them to the mitochondria. The metabolic activity of the cell is related to the number of cristae and the number of mitochondria within a cell. Cells with high energetic activity, such as heart muscle, have many well developed mitochondria. New mitochondria are formed from preexisting mitochondria when they grow and divide.

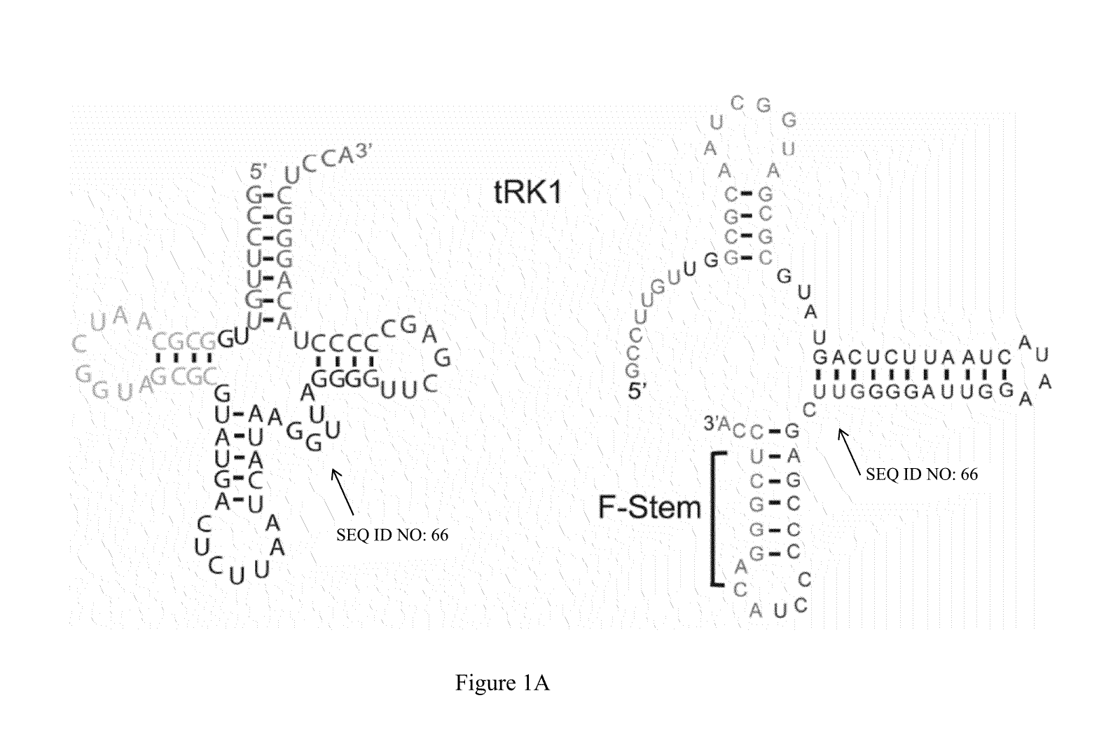

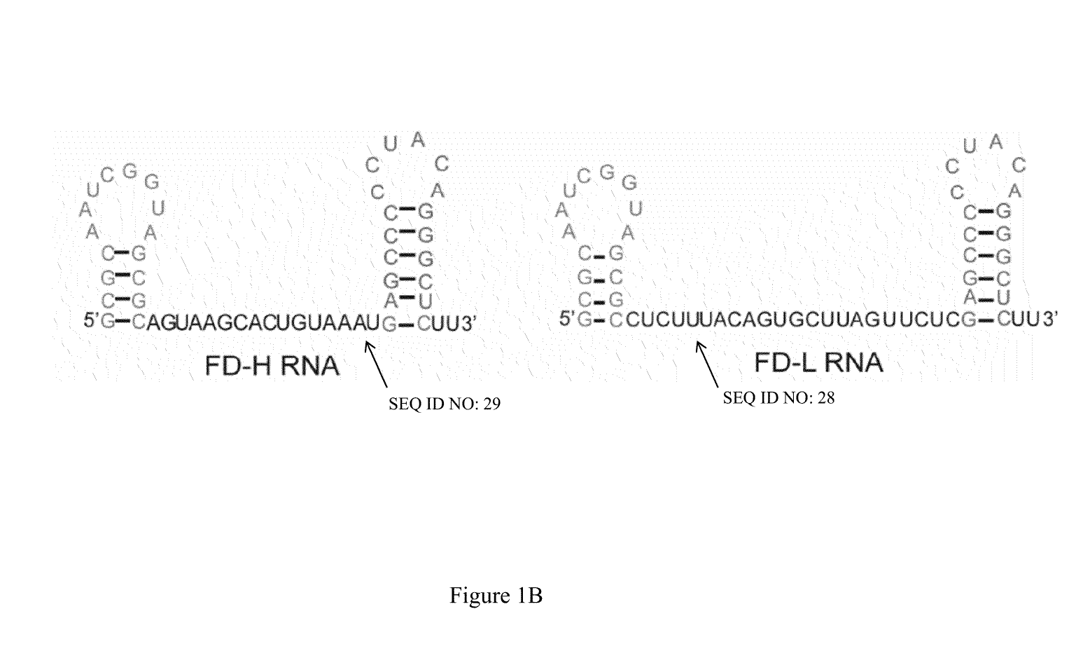

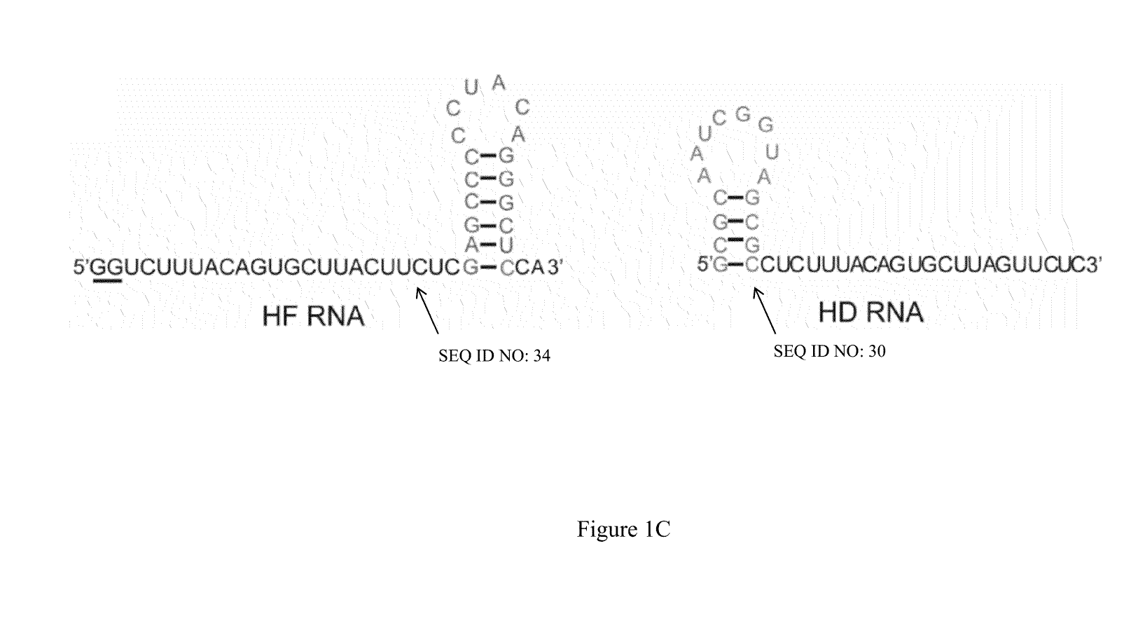

In some embodiments of the present disclosure, a mitochondrial vector comprising a first nucleic acid sequence derived from tRK1. In yeast Saccharomyces cerevisiae, the cytosolic tRNA^{Lys}CUU (further referred to as tRK1) is transcribed from nuclear genes and then unequally redistributed between the cytosol (97-98%) and mitochondria (2-3%). The mitochondrial pathway was shown to be essential for mitochondrial translation at elevated temperatures, when the mtDNA-encoded isoacceptor tRNA^{Lys}UUU becomes under-modified at the wobble position of the anticodon and loses its capacity to recognize the lysine AAG codon. The mitochondrial targeting of tRK1 in yeast in vitro and in vivo was shown to depend on the cytosolic precursor of mitochondrial lysyl-tRNA synthetase (preMSKlp), which serves as a carrier, and the glycolytic enzyme enolase (Eno2p). Analysis of conformational rearrangements in the RNA by in-gel FRET approach permitted to demonstrate that binding to the protein factors and the subsequent RNA import require formation of an alternative structure, different from the classic L-form tRNA model. In the complex with Eno2p, tRK1 adopts a particular conformation characterized by bringing together the 39-end and the TYC loop and forming a structure referred to as F-hairpin (FIG. 1A). The data suggested that only those RNAs that are able to form a stable alternative F-stem proceed to the mitochondrial import pathway involving specific interactions with the carrier protein, preMSKlp, and membrane receptors.

Exploiting these data, a set of small RNA molecules based on either the D or F-hairpin sequence or both, with a surprisingly high efficiency of import not only into yeast but also into human mitochondria in vitro and in vivo, have been constructed. The mitochondrial import of the small RNA molecules are not dependent on preKARS2 (contrary to the full-size tRK1). This opened a possibility to design a new vector system capable to target therapeutic oligonucleotides, in particular oligoribonucleotides, into deficient human mitochondria. In some embodiments of the present disclosure, the nucleic acid sequence derived from tRK1 is two alternative structures D or F-hairpin (FIG. 1).

The present invention relates to a mitochondrial vector comprising either the D-arm or the F-hairpin sequence or a derivative thereof, which is called the first nucleic acid sequence, and a second nucleic acid sequence. The mitochondrial vector includes the D-arm sequence or a derivative thereof, or the F-hairpin sequence or a derivative thereof, but not both D-arm and F-hairpin sequences.

In one embodiment, the D-arm sequence consists of or consists essentially of the nucleotides of SEQ ID NO: 1 (5′GCGCAAUCGGUAGCGC3′) or a sequence having at least 75%, 80%, 85%, 90%, 93%, 95%, 96%, 97%, 98% or 99% sequence identity thereto wherein said sequence. In particular, the D-arm sequence can have 1, 2, 3 or 4 nucleotide substitution, deletion or addition with respect to the sequence of SEQ ID NO: 1. Preferably, it may have 1 or 2 nucleotide substitution, deletion or addition with respect to the sequence of SEQ ID NO: 1. In a particular embodiment, the D-arm sequence consists of the nucleotides of SEQ ID NO: 1. By “consists essentially of” is intended that the D-arm sequence may comprise 1, 2, 3 or 4 additional nucleotides.

In this embodiment, the D-arm sequence is at the 5′ end of the mitochondrial vector or is about at the 5′ end. Optionally, the mitochondrial vector may comprise additional 1-5 nucleotides at the 5′ end, for instance 1, 2, 3, 4 or 5. Preferably, the D-arm sequence is at the 5′ end of the mitochondrial vector and there are no additional nucleotides at the 5′ end.

In a preferred embodiment, the D-arm sequence is made of or essentially made of ribonucleotides. By “essentially made of” is intended that it may include 1 or 2 deoxyribonucleotides.

Optionally, the D-arm sequence may comprise nucleotide modifications. For instance, it may comprise backbone modifications such as methyphophonate, phosphorothioate, phosphordithioate, formacetal, 3′-thioformacetal, sulfone, or sulfamate. More preferably, it may comprise 1-3 phosphorothioates at the 5′ end of the D-arm sequence. In addition or alternatively, the 5′ end may comprise a 5′-5′ inverted nucleotide, in particular a 5′-5′ inverted thymidine.

In this embodiment, the D-arm sequence is preferably linked to the second nucleic acid sequence at its 3′ end. It can be linked directly or through a linker.

In another embodiment, the F-hairpin sequence consists of or consists essentially of the nucleotides of SEQ ID NO: 5 (5′GAGCCCCCUACAGGGCUC3′) or a sequence having at least 75%, 80%, 85%, 90%, 93%, 95%, 96%, 97%, 98% or 99% sequence identity thereto wherein said sequence. In particular, the F-hairpin sequence can have 1, 2, 3 or 4 nucleotide substitution, deletion or addition with respect to the sequence of SEQ ID NO: 5. Preferably, it may have 1 or 2 nucleotide substitution, deletion or addition with respect to the sequence of SEQ ID NO: 5. In a particular embodiment, the F-hairpin sequence consists of the nucleotides of SEQ ID NO: 5. By “consists essentially of” is intended that the D-arm sequence may comprise 1, 2, 3 or 4 additional nucleotides.

In this embodiment, the F-hairpin sequence is at the 3′ end of the mitochondrial vector or is about at the 3′ end. Optionally, the mitochondrial vector may comprise additional 1-5 nucleotides at the 3′ end, for instance 1, 2, 3, 4 or 5. Preferably, the F-hairpin sequence is either at the 3′ end of the mitochondrial vector and there is no additional nucleotide at the 3′ end. More preferably, the F-hairpin sequence is at the 3′ end of the mitochondrial vector but with two additional nucleotide at the 3′ end.

In a preferred embodiment, the F-hairpin sequence is made of or essentially made of ribonucleotides. By “essentially made of” is intended that it may include 1 or 2 deoxyribonucleotides.

Optionally, the F-hairpin sequence may comprise nucleotide modifications. For instance, it may comprise backbone modifications such as methyphophonate, phosphorothioate, phosphordithioate, formacetal, 3′-thioformacetal, sulfone, or sulfamate. More preferably, it may comprise 1-3 phosphorothioates at the 3′ end of the F-hairpin sequence. Optionally, it may further comprise a 3′-3′ inverted nucleotide at the 3′ end, in particular a 3′-3′ inverted thymidine.

In this embodiment, the F-hairpin sequence is preferably linked to the second nucleic acid sequence at its 5′ end. It can be linked directly or through a linker.

The mitochondrial vector does not comprise both D-arm and F-hairpin. In addition, the mitochondrial vector does not comprise the combination of a first nucleic acid sequence and a second nucleic acid sequence that can be found in nature.

The second nucleic acid sequence can be any nucleic acid sequence of interest, in particular any sequence for which its importation within the mitochondria may have an interest. The interest can be a therapeutic interest or a scientific interest for research. For instance, the nucleic acid sequence can be a sequence capable of modulating the replication or expression of a mitochondrial gene. In particular, the sequence is capable of decreasing the replication or expression of the mitochondrial gene. For instance, the second nucleic acid sequence can be antisense molecule.

In one embodiment, the second nucleic acid sequence has a size of less than 50 nucleotides, preferably between 10 and 30 nucleotides, more preferably between 15 and 25 nucleotides.

In some embodiments of the present disclosure, a second nucleic acid sequence may be a wild-type sequence of a mitochondrial gene or a fragment thereof or a complement thereof or an altered sequence, i.e. a wild-type sequence having one or more mutations, substitutions, additions and/or deletions. For instance, the mutation can be a point mutation and the additions and/or deletions can be of at least 1, 2, 5, or 10 nucleotides.

Optionally, the second nucleic acid sequence may comprise one or several mismatches when compared to the targeted mitochondrial sequence. More preferably, the second nucleic acid sequence is fully complementary of the targeted mitochondrial sequence. The second nucleic acid molecule can comprise the L-strand sequence or the H-strand sequence of a segment of the mitochondrial gene.

In a preferred embodiment, the second nucleic acid sequence is designed so as to be specific of one of the wild-type or mutated sequence. In other words, the second nucleic acid sequence has a binding which is able to discriminate between the wild-type sequence and the mutated sequence. Several methods are available to the person skilled in the art for designing such a second nucleic acid sequence, including in silico and in vitro tools. In addition, the second nucleic acid sequence is designed for having a low probability of alternative structure in the context of the mitochondrial vector. More specifically, the second nucleic acid sequence has a low probability to adopt a secondary structure or with the D-arm or F-hairpin sequence. When the second nucleic acid sequence targets a mutated mitochondrial gene, the sequence complementary to the mutation is preferably located within the center of the second nucleic acid sequence.

Exemplary mtDNA mutations that can be addressed by the present disclosure include but are not limited to: tRNAleu-A3243G, A3251G, A3303G, T3250C, T3271C and T3394C; tRNAlys-A8344G, G11778A, G8363A, T8356C; ND1-G3460A; ND4-A10750G, G14459A; ND6-T14484A; 12S rRNA-A1555G; MTTS2-C12258A; ATPase 6-T8993G, T8993C; tRNASer(UCN)-T7511C; 11778 and 14484, LHON mutations as well as mutations or deletions in ND2, ND3, NDS, cytochrome b, cytochrome oxidase I-III, and ATPase 8. Other mtDNA mutations are disclosed on MITOMAP, which reports published and unpublished data on human mitochondrial DNA variation, www.mitomap.org/MITOMAP. All type of mutations and rearrangements can be addressed if the mutation in heteroplasmic.

Optionally, the second nucleic acid sequence comprises deoxyribonucleotides, ribonucleotides or a mixture thereof. In a particular embodiment, the second nucleic acid sequence is made of or essentially made of deoxyribonucleotides.

The second nucleic acid sequence may further comprise modified nucleotides including, but not limited to, 5-fluorouracil, 5-bromouracil, 5-chlorouracil, 5-iodouracil, hypoxanthine, xanthine, 4-acetylcytosine, 5-(carboxyhydroxylmethyl) 5-carboxymethylaminomethyl-2-thiouridine, 5-carboxymethylaminomethyluracil, dihydrouracil, [beta]-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, [beta]-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. In a preferred embodiment, the second nucleic acid sequence comprises 2′-O-methylated ribonucleotide(s), more preferably 2′-O-methylated pyrimidine(s), and more specifically 2′-O-methylated uracil(s). In a preferred embodiment, the modified nucleotides are introduced at the nuclease sensitive sites.

Optionally, when the second nucleic acid sequence is at the 3′ end of the mitochondrial vector, it may comprise a 3′-3′ inverted nucleotide (e.g., thymidine) and, when the second nucleic acid sequence is at the 5′ end of the mitochondrial vector, it may comprise a 5 ‘-5’ nucleotide (e.g., thymidine).

Optionally, the second nucleic acid sequence may comprise backbone modifications such as methyphophonate, phosphorothioate, phosphordithioate, formacetal, 3′-thioformacetal, sulfone, or sulfamate. More preferably, it may comprise 1-3 phosphorothioates at the free end of the F second nucleic acid sequence. Especially, when the second nucleic acid sequence is at the 3′ end of the mitochondrial vector, it may comprise 1-3 phosphorothioates at the 3′ end and, when the second nucleic acid sequence is at the 5′ end of the mitochondrial vector, it may comprise 1-3 phosphorothioates at the 5′ end. Non-exhaustive examples of second nucleic acid sequences of interest for treating a mitochondrial disease include:

(SEQ ID NO: 6)
UCUUUACAGUGCUUAGUUCUC;
its DNA equivalent
(SEQ ID NO: 7)
TCTTTACAGTGCTTAGTTCTC;
and a fragment of the DNA
(SEQ ID NO: 8)
equivalent TTACAGTGCTTA
or its complementary sequence
(SEQ ID NO: 9)
AGTAAGCACTGTA;
(SEQ ID NO: 10)
AUGUGGCCUUUGGAGU
or
(SEQ ID NO: 11)
GAUGAUGUGGCCUUUGGAGU
or
(SEQ ID NO: 12)
AUGAUGUGGCCUUUGGAGUAGAAAC
or
(SEQ ID NO: 13)
ACUCCAAAGGCCACAU
or
(SEQ ID NO: 14)
ACUCCAAAGGCCACAUCAUC
or
(SEQ ID NO: 15)
GUUUCUACUCCAAAGGCCACAUCAUC
or
(SEQ ID NO: 16)
GATGATGTGGCCTTTGGAGT
or
(SEQ ID NO: 17)
ACTCCAAAGGCCACATC.

Preferably, the mitochondrial vector is between 26 to 48 nucleotides in length, more preferably between 30 to 40 nucleotides in length.

In one embodiment, the mitochondrial vector comprises a loop (e.g., either a D-arm or a F-hairpin) and a second nucleic acid sequence which is linear. The mitochondrial vector can be conjugated with a delivery system which facilitates endocytosis or cellular uptake. The delivery system is preferably conjugated at the free end of the second nucleic acid sequence, the end which is not linked to the D-arm or the F-hairpin.

In particular, the delivery system may be lipophilic molecules such as cholesterol, single or double chain fatty acids, or ligands which target cell receptor enabling receptor mediated endocytosis, such as folic acid and folate derivatives or transferrin (Goldstein et al., Ann. Rev. Cell Biol. 1985 1:1-39; Leamon & Lowe, Proc Natl Acad Sci USA. 1991, 88: 5572-5576.). Fatty acids may be saturated or unsaturated and be in C₄-C₂₈, preferably in C₁₄-C₂₂, still more preferably being in C₁₈ such as oleic acid or stearic acid. In particular, fatty acids may be octadecyl or dioleoyl. Fatty acids may be found as double chain forms linked with in appropriate linker such as a glycerol, a phosphatidylcholine or ethanolamine and the like. As used herein, the term “folate” is meant to refer to folate and folate derivatives, including pteroic acid derivatives and analogs. The analogs and derivatives of folic acid suitable for use in the present invention include, but are not limited to, antifolates, dihydrofolates, tetrahydrofolates, folinic acid, pteropolyglutamic acid, 1-deza, 3-deaza, 5-deaza, 8-deaza, 10-deaza, 1,5-deaza, 5,10 dideaza, 8,10-dideaza, and 5,8-dideaza folates, antifolates, and pteroic acid derivatives. Additional folate analogs are described in US2004/242582. The delivery system may be tocopherol, sugar such as galactose and mannose and their oligosaccharide, peptide such as RGD and bombesin, and proteins such as integrin. Accordingly, the delivery system may be selected from the group consisting of single or double chain fatty acids, folates and cholesterol. More preferably, the molecule delivery system is cholesterol or its derivatives.

In one specific embodiment, the mitochondrial vector consists of or consists essentially of the nucleotides of SEQ ID NO: 2 (5′GCGCAAUCGGUAGCGCCUCUUUACAGUGCUUAGUUCUC3′) or a sequence having at least 75%, 80%, 85%, 90%, 93%, 95%, 96%, 97%, 98% or 99% sequence identity thereto wherein said sequence. This mitochondrial vector is used for deletion strategy for treating a mitochondrial disease caused by a mutation in a gene in the mitochondrial genome. More particular, this mitochondrial vector is suitable for treating Kearns-Sayre syndrome (KSS) in a subject, in particular subject having a deletion spanned from 8363 to 15,438. In another specific embodiment, the mitochondrial vector consists of or consists essentially of the nucleotides of SEQ ID NO: 3 (5′GCGCAAUCGGUAGCGCGATGATGTGGCCTTTGGAGT3′) or a sequence having at least 75% 80%, 85%, 90%, 93%, 95%, 96%, 97%, 98% or 99% sequence identity thereto wherein said sequence. This mitochondrial vector is used for point mutation strategy for treating a mitochondrial disease caused by a mutation in a gene in the mitochondrial genome. More particular, this mitochondrial vector is suitable for treating a mitochondrial disease caused in a subject, in particular a mitochondrial disease caused a point mutation in the mtDNA ND5 gene, especially A13514>G mutation, such as MELAS-like and Leigh syndromes.

In a further specific embodiment, the mitochondrial vector consists of or consists essentially of the nucleotides of SEQ ID NO: 4 (5′GCGCAAUCGGUAGCGCACTCCAAAGGCCACATCATC3′) or a nucleotides having at least 75%, 80%, 85%, 90%, 93%, 95%, 96%, 97%, 98% or 99% sequence identity thereto wherein said sequence. This mitochondrial vector is used for point mutation strategy for treating a mitochondrial disease caused by a mutation in a gene in the mitochondrial genome. More particular, this mitochondrial vector is suitable for treating a mitochondrial disease caused in a subject, in particular a mitochondrial disease caused a point mutation in the mtDNA ND5 gene, especially A13514>G mutation, such as MELAS-like and Leigh syndromes.

In an additional embodiment, the mitochondrial vector consists of or consists essentially of the nucleotides of SEQ ID NO: 34 (5 ‘GGUCUUUACAGUGCUUACUUCUCGAGCCCCCUACAGGGCUCCA3’) or a sequence having at least 75%, 80%, 85%, 90%, 93%, 95%, 96%, 97%, 98% or 99% sequence identity thereto wherein said sequence. This D-hairpin sequence is used for deletion strategy for treating a mitochondrial disease caused by a mutation in a gene in the mitochondrial genome. More particular, this mitochondrial vector is suitable for treating Kearns-Sayre syndrome (KSS) in a subject, in particular subject having a deletion spanned from 8363 to 15,438.

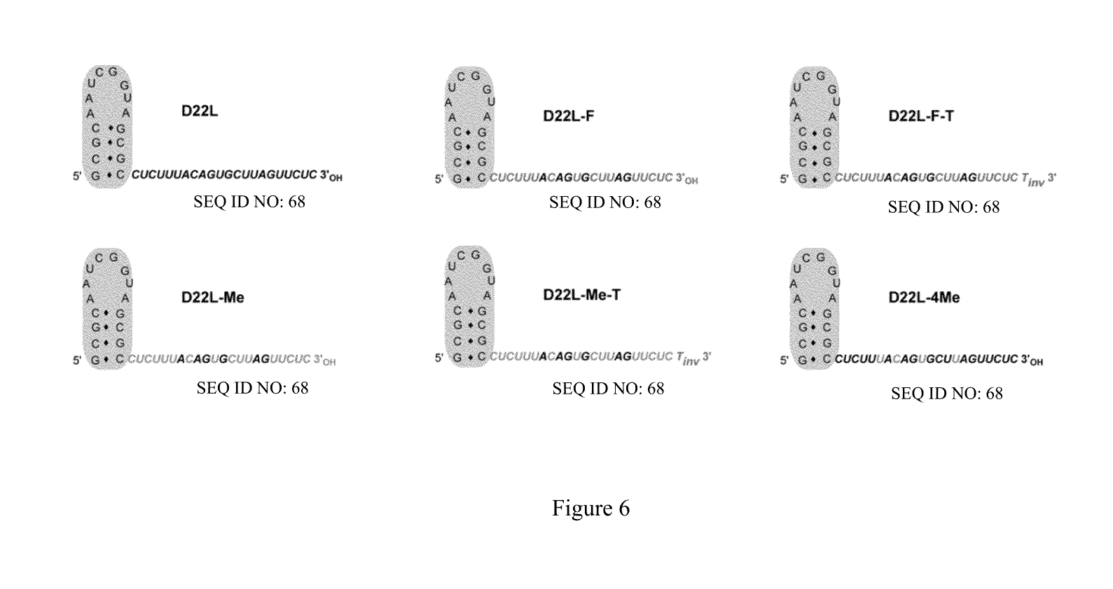

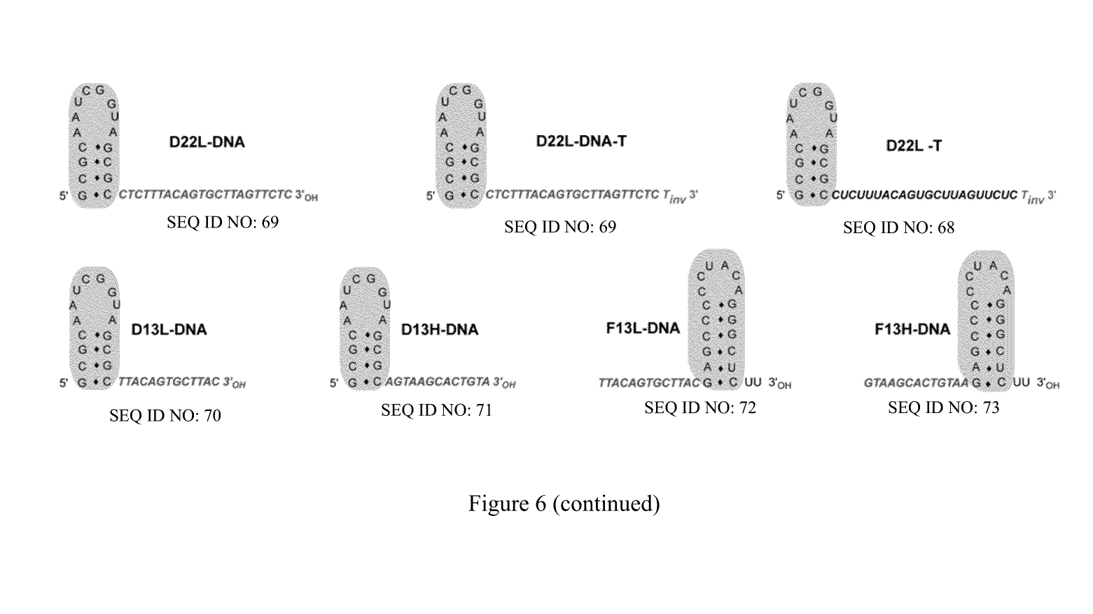

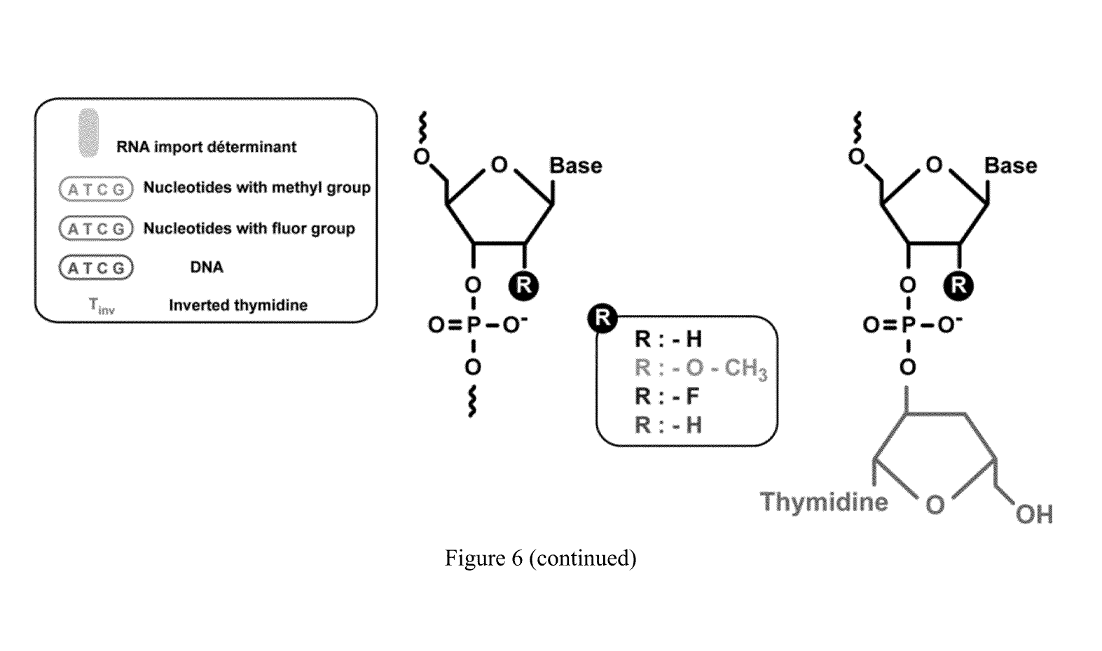

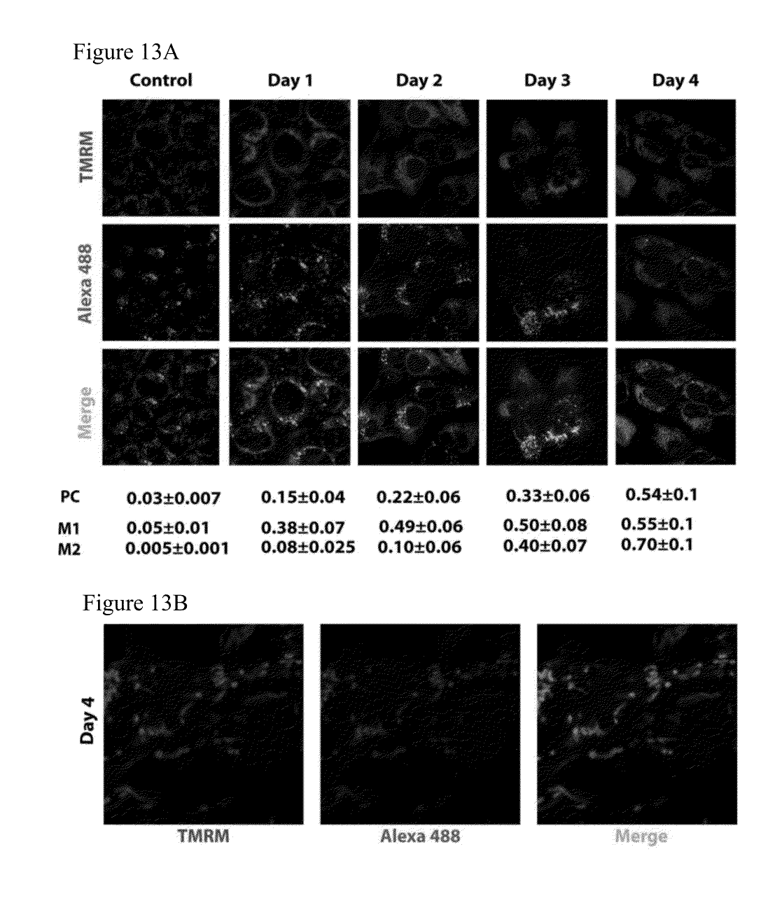

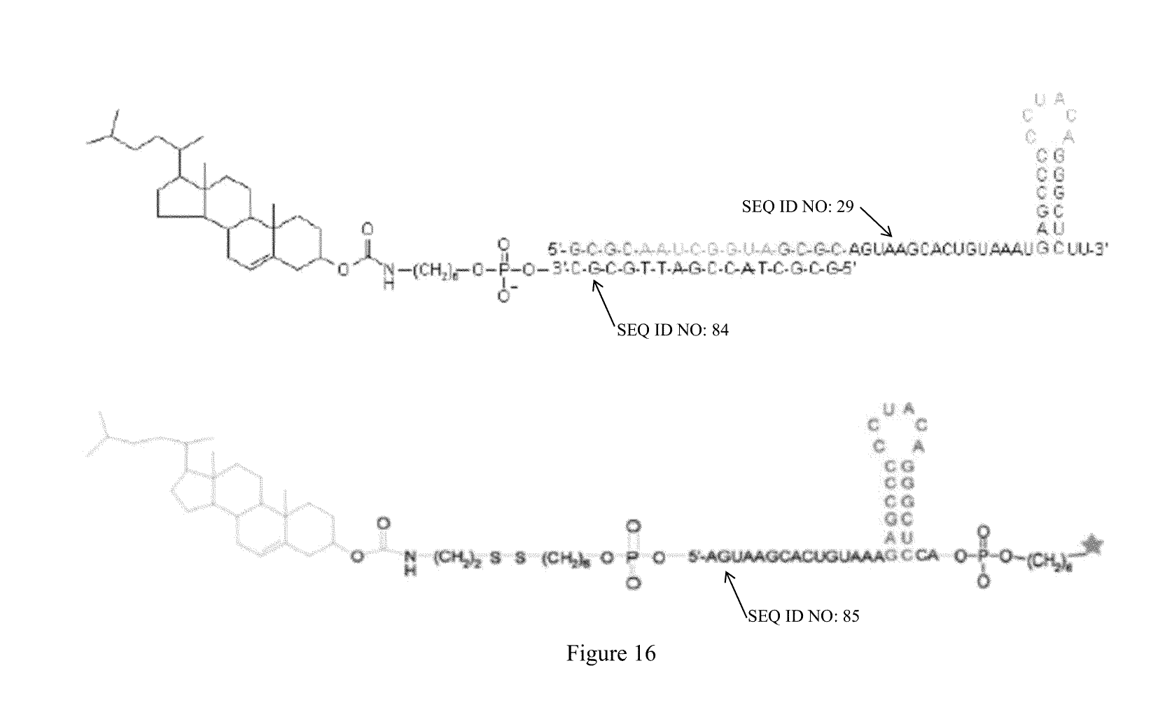

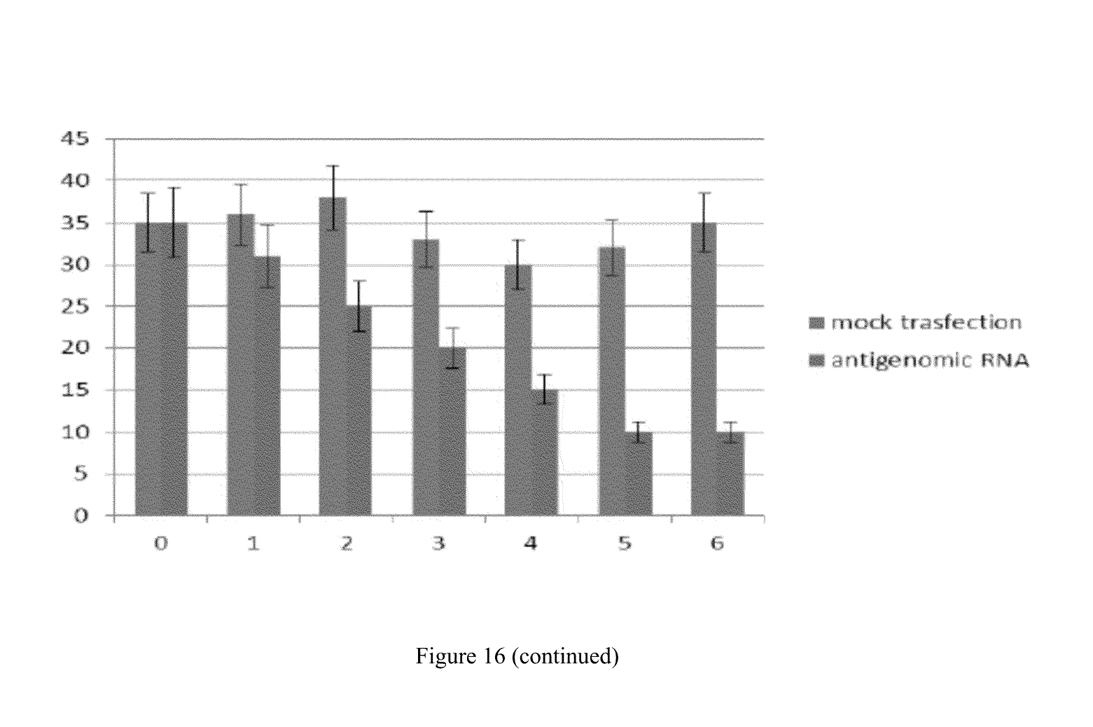

In addition, the present invention relates to any mitochondrial vector disclosed in the present application, such as HF and HD of FIG. 1, the vector of FIG. 6, D20H-DNA and D20L-DNA of FIG. 13 and the vectors of FIG. 16.

In some embodiments of the present disclosure, a mitochondrial vector is used for therapy of mitochondrial diseases.

Given the importance of mitochondria in human disease, cell proliferation, cell death, and aging, the present disclosure also encompasses the manipulation of the mitochondrial genome to supply the means by which known mitochondrial diseases (LHON, MELAS, MERRF, KSS, etc.) and putative mitochondrial diseases (aging, Alzheimer's, Parkinson's, Diabetes, Heart Disease) can be treated.

Mitochondrial diseases result from failures of the mitochondria, specialized compartments present in every cell of the body except red blood cells. Cell injury and even cell death are result from mitochondrial failure.

Thus, embodiments of the present disclosure are directed to treating a mitochondrial disease by introducing a mitochondrial vector comprising a first nucleic acid sequence of the present invention derived from tRK1, directly or indirectly linked to a second nucleic acid sequence, which may be a wild-type mitochondrial DNA sequence or an altered mitochondrial DNA sequence. The present disclosure encompasses manipulating and alteration of proportion between mutant and wild-type mitochondrial genomes of the mammalian cell to treat diseases caused by mitochondrial genetic defects or abnormalities.

Exemplary mitochondrial diseases include but are not limited to: Alpers Disease; Barth syndrome; p-oxidation defects; carnitine-acyl-carnitine deficiency; carnitine deficiency; co-enzyme Q10 deficiency; Complex I deficiency; Complex II deficiency; Complex III deficiency; Complex IV deficiency; Complex V deficiency; cytochrome c oxidase (COX) deficiency; Chronic Progressive External Ophthalmoplegia Syndrome (CPEO); CPT I Deficiency; CPT II deficiency; Glutaric Aciduria Type II; lactic acidosis; Long-Chain Acyl-CoA Dehydrongenase Deficiency (LCAD); LCHAD; mitochondrial cytopathy; mitochondrial DNA depletion; mitochondrial encephalopathy; mitochondrial myopathy; Mitochondrial Encephalomyopathy with Lactic Acidosis and Stroke like episodes (MELAS); Myoclonus Epilepsy with Ragged Red Fibers (MERRF); Maternally Inherited Leigh's Syndrome (MILS); Myogastrointestinal encephalomyopathy (MNGIE); Neuropathy, ataxia and retinitis pigmentosa (NARP); Leber's Hereditary Optic Neuropathy (LHON); Progressive external ophthalmoplegia (PEO); Pearson syndrome; Kearns-Sayre syndrome (KSS); Leigh's syndrome; intermittent dysautonomia; pyruvate carboxylase deficiency; pyruvate dehydrogenase deficiency; respiratory chain mutations and deletions; Short-Chain Acyl-GoA Dehydrogenase Deficiency (SCAD); SCHAD; and Very Long-Chain Acyl-CoA Dehydrongenase Deficiency (VLCAD); Pearson's Disease.

Some mitochondrial diseases are a result of problems in the respiratory chain in the mitochondria. The respiratory chain consists of four large protein complexes: I, II, III and IV (cytochrome c oxidase, or COX), ATP synthase, and two small molecules that ferry around electrons, coenzyme Q10 and cytochrome c. The respiratory chain is the final step in the energy-making process in the mitochondrion where most of the ATP is generated. Mitochondrial encephalomyopathies that can be caused by deficiencies in one or more of the specific respiratory chain complexes include MELAS, MERFF, Leigh's syndrome, KSS, Pearson, PEO, NARP, MILS and MNGIE. The mitochondrial respiratory chain is made up of proteins that come from both nuclear and mtDNA. Although only 13 of roughly 100 respiratory chain proteins come from the mtDNA, these 13 proteins contribute to every part of the respiratory chain except complex 11, and 24 other mitochondrial genes are required just to manufacture those 13 proteins. Thus, a defect in either a nuclear gene or one of the 37 mitochondrial genes can cause the respiratory chain to break down.

Depending on which cells are affected, symptoms may include loss of motor control, muscle weakness and pain, gastrointestinal disorders and swallowing difficulties, poor growth, cardiac disease, liver disease, diabetes, respiratory complications, seizures, visual/hearing problems, lactic acidosis, developmental delays and susceptibility to infection.

One embodiment of the present disclosure provides a method for restoring or increasing respiratory chain function in host cells including introducing a mitochondrial vector comprising a first nucleic acid sequence of the present invention derived from tRK1, directly or indirectly linked to a second nucleic acid sequence, which may be a wild-type mitochondrial DNA sequence or an altered mitochondrial DNA sequence.

In some embodiments of the present disclosure, a mitochondrial vector is conjugated with a delivery system. Examples of delivery system include but are not limited to lipid conjugated oligonucleotides (FIG. 16), conjugated or not with a biodegradable link.

In some embodiments of the present disclosure, a mitochondrial vector in pharmaceutical composition, and with a pharmaceutically acceptable carrier or excipient.

The compositions provided herein may be administered in a physiologically acceptable carrier to a host. Preferred methods of administration include systemic or direct administration to a cell. The compositions can be administered to a cell or patient, as is generally known in the art for gene therapy applications. In gene therapy applications, the compositions are introduced into cells in order to transfect mitochondria “Gene therapy” includes both conventional gene therapy where a lasting effect is achieved by a single or repeated treatment, and the administration of gene therapeutic agents, which involves the one or more time administration of therapeutically effective RNA or RNA containing chimeric molecules.

Therapeutic formulations are prepared for storage by mixing the active ingredient having the desired degree of purity with optional physiologically acceptable carriers, excipients or stabilizers, in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamin, asparagine, arginine or lysine; monosaccharides, disaccharides and other carbohydrates including glucose, mannose, or dextrins; cheating agents such as EDTA; sugar alcohols such as mannitol or sorbitol; salt-forming counterions such as sodium; and/or nonionic surfactants such as Tween, Pluronics or PEG.

The compositions of the present disclosure can be administered parenterally. As used herein, “parenteral administration” is characterized by administering a pharmaceutical composition through a physical breach of a subject's tissue. Parenteral administration includes administering by injection, through a surgical incision, or through a tissue-penetrating non-surgical wound, and the like. In particular, parenteral administration includes subcutaneous, intraperitoneal, intravenous, intraarterial, intramuscular, intrasternal injection, and kidney dialytic infusion techniques.

Parenteral formulations can include the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Parenteral administration formulations include suspensions, solutions, emulsions in oily or aqueous vehicles, pastes, reconsitutable dry (i. e. powder or granular) formulations, and implantable sustained-release or biodegradable formulations. Such formulations may also include one or more additional ingredients including suspending, stabilizing, or dispersing agents. Parenteral formulations may be prepared, packaged, or sold in the form of a sterile injectable aqueous or oily suspension or solution. Parenteral formulations may also include dispersing agents, wetting agents, or suspending agents described herein. Methods for preparing these types of formulations are known. Sterile injectable formulations may be prepared using non-toxic parenterally-acceptable diluents or solvents, such as water, 1, 3-butane diol, Ringer's solution, isotonic sodium chloride solution, and fixed oils such as synthetic monoglycerides or diglycerides. Other parentally-administrable formulations include microcrystalline forms, liposomal preparations, and biodegradable polymer systems. Compositions for sustained release or implantation may include pharmaceutical acceptable polymeric or hydrophobic materials such as emulsions, ion exchange resins, sparingly soluble polymers, and sparingly soluble salts.

Pharmaceutical compositions may be prepared, packaged, or sold in a buccal formulation. Such formulations may be in the form of tablets, powders, aerosols, atomized solutions, suspensions, or lozenges made using known methods, and may contain an orally dissolvable or degradable composition and/or one or more additional ingredients as described herein.

As used herein, “additional ingredients” include one or more of the following: excipients, surface active agents, dispersing agents, inert diluents, granulating agents, disintegrating agents, binding agents, lubricating agents, sweetening agents, flavoring agents, coloring agents, preservatives, physiologically degradable compositions (e. g., gelatin), aqueous vehicles, aqueous solvents, oily vehicles and oily solvents, suspending agents, dispersing agents, wetting agents, emulsifying agents, demulcents, buffers, salts, thickening agents, fillers, emulsifying agents, antioxidants, antibiotics, antifungal agents, stabilizing agents, and pharmaceutically acceptable polymeric or hydrophobic materials. Other “additional ingredients” which may be included in the pharmaceutical compositions are known.

Dosages and desired concentrations of the modified vectors disclosed herein in pharmaceutical compositions of the present disclosure may vary depending on the particular use envisioned. The determination of the appropriate dosage or route of administration is well within the skill of an ordinary physician. Animal experiments provide reliable guidance for the determination of effective doses for human therapy.

In a particular embodiment, a kit, or pharmaceutical pack, comprises the mitochondrial vector and one or more of the ingredients of the pharmaceutical compositions of the invention. In addition, the compositions of the present invention may be employed in conjunction with other therapeutic compositions.

### EXAMPLES

### Induced tRNA Import into Human Mitochondria: Implication of a Host Aminoacyl-tRNA-Synthetase

### Example 1

### PreKARS2 Binds tRK1 and Artificial Importable RNA Molecules

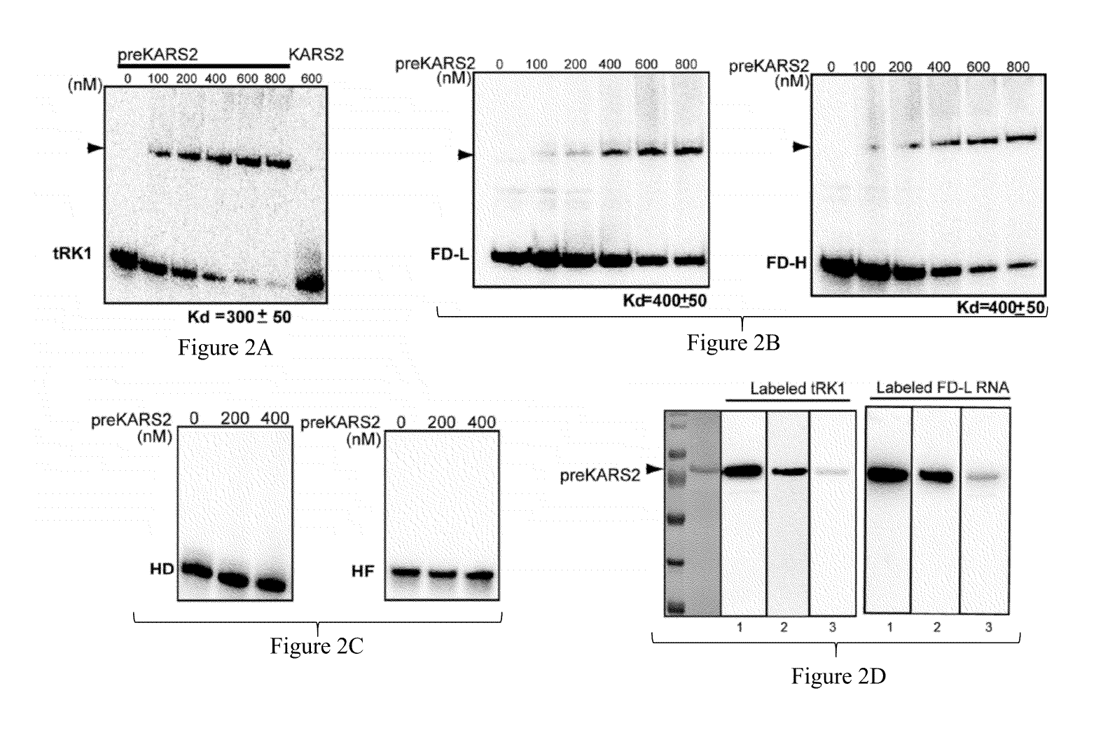

To study the implication of the cytosolic precursor of human mitochondrial lysyl-tRNA synthetase (preKARS2) in the mitochondrial import of the yeast cytosolic tRNA^{Lys} CUU (tRK1), inventors first analysed the interaction of the recombinant preKARS2 with a T7-transcript of tRK1 by EMSA (FIG. 2), using labeled RNA and increasing concentrations of the protein, as described (Entelis N, Brandina I, Kamenski P, Krasheninnikov I A, Martin R P, et al. (2006)). A glycolytic enzyme, enolase, is recruited as a cofactor of tRNA targeting toward mitochondria in Saccharomyces cerevisiae. Genes Dev 20: 1609-1620. The apparent Kd of the complex was estimated as 300+/250 nM. Thus, the affinity of preKARS2 to tRK1 is only slightly lower than that of its yeast homolog, preMSKlp, with the apparent Kd previously evaluated as 280+/260 nM. Noteworthy, the recombinant protein lacking the mitochondrial targeting presequence predicted by Mitoprot and thus corresponding to the mature mitochondrial enzyme KARS2 was not able to interact with tRK1 (FIG. 2A). This finding parallels our previous study suggesting a particular way of interaction between tRK1 and yeast preMSKlp which does not lead to the tRNA aminoacylation.

Previous analysis of RNA aptamers imported into human mitochondria permitted us to design short synthetic RNAs comprising two domains of the tRK1 alternative structure (FIG. 1 A, B) and characterized by a high efficiency of mitochondrial targeting [12,17]. The molecules referred to as FD-L and FD-H, containing the D-arm and F-hairpin parts of tRK1 separated by 17-22 nucleotide stretches, were able to form complexes with the recombinant preKARS2 with the apparent Kd of 400+/250 nM, indicating a lower but still important affinity to preKARS2 (FIG. 2B). The specificity of the interaction was verified by North-Western hybridization in the presence of specific and nonspecific competitors (FIG. 2D). The data show that 306 molar excess of cold E. coli rRNA only partially decreased the interaction of preKARS2 with labeled tRK1 and FD-L RNA, whereas the 106 molar excess of cold FD-R RNA completely abolished this interaction.

To study more precisely the role of each of the two stem-loop RNA domains, we constructed truncated FD-L RNA molecules (FIG. 1C) lacking either the D-arm (HF RNA) or the F-hairpin (HD RNA) of tRK1. Neither molecule was able to interact with preKARS2 (FIG. 2C), indicating the importance of the simultaneous presence of the D-arm and the F-hairpin for the RNA affinity to preKARS2.

### Example 2

### PreKARS2 can Direct the RNA Import into Isolated Human Mitochondria

Previously, inventors suggested that preKARS2 might replace preMsk1p in the import of tRK1 into human mitochondria [19]. To demonstrate this directly, the in vitro import test was performed by incubating the proteins and the labelled RNA with purified mitochondria from HepG2 cells, as described [22]. Inventors tested the recombinant preKARS2 in combination with rabbit enolase, since our previous study of the tRK1 import into yeast mitochondria had shown that yeast enolase recognizes the imported tRNA and favours its binding to preMSKlp [10].

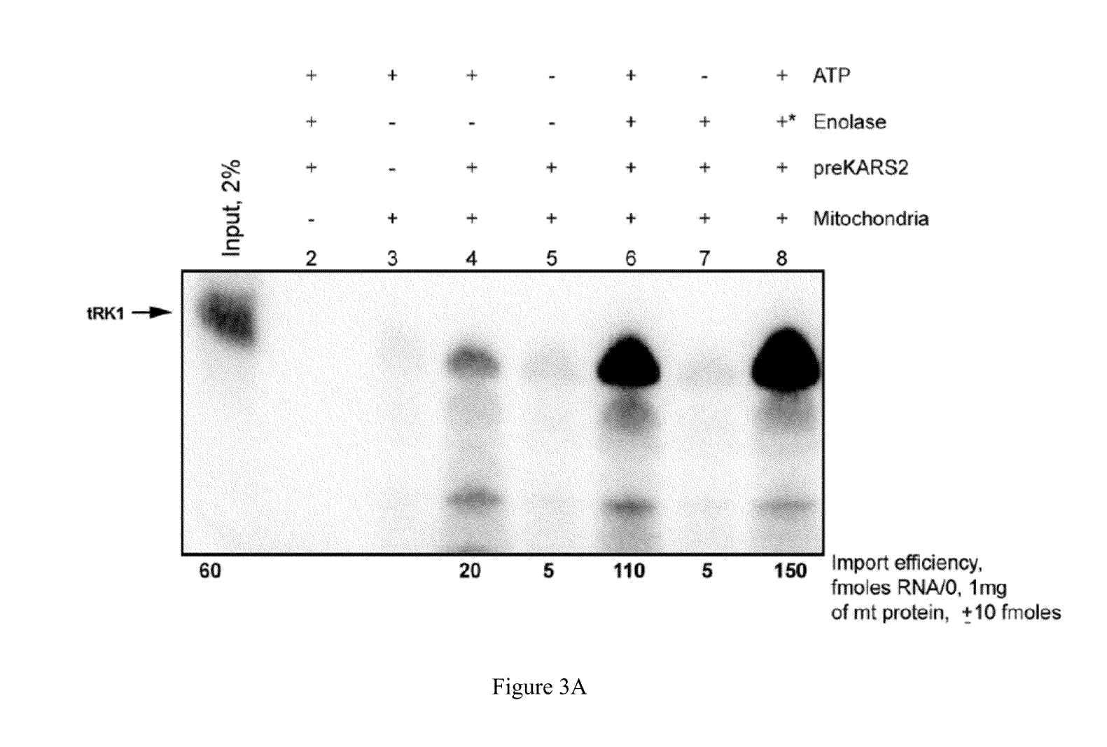

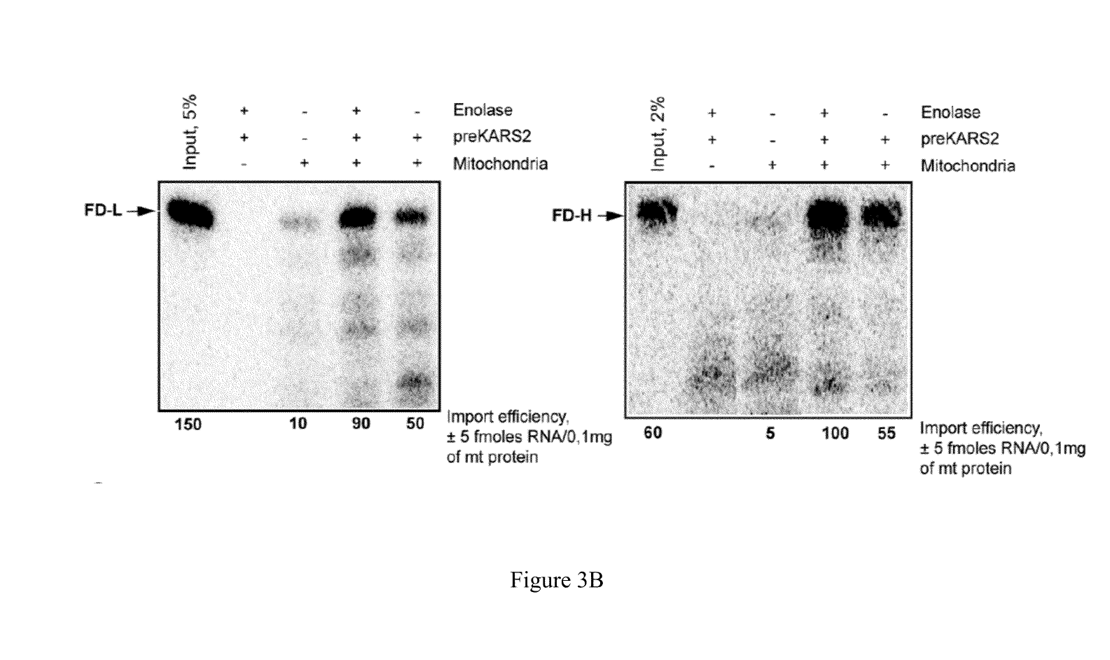

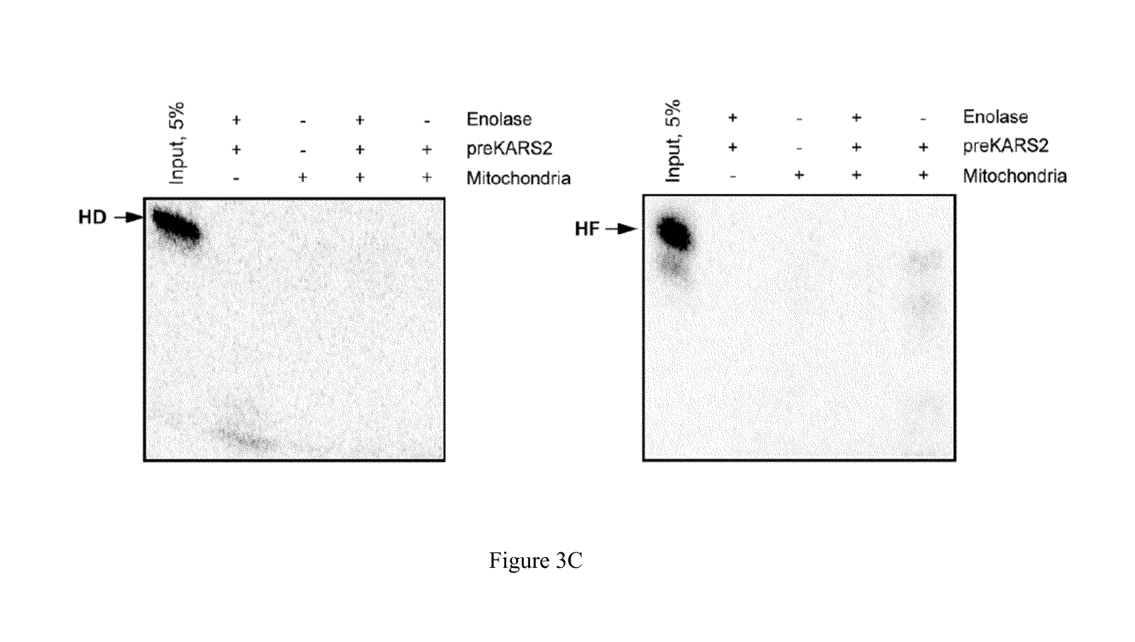

Purified human mitochondria were not able to internalize the external tRK1 in the absence of protein factors (FIG. 3). Control reactions without mitochondria or in the absence of ATP (FIG. 3A) demonstrate that the proteins do not protect the RNA from nuclease digestion. Upon addition of mitochondria and the recombinant preKARS2, a portion of tRK1 and the small artificial RNAs FD-L and FD-H has been protected from nuclease degradation (FIG. 3 A, B), thus indicating their import into the mitochondria. The amount of the imported RNA was determined by comparison of the band density of the protected full-size RNA isolated from the mitoplasts after the import assay with that of an aliquot of the input labelled RNA, as shown in FIG. 3. As it was demonstrated previously [18,19], only a minor fraction (1-5%) of the tRK1 added to the import mixture is transported into the isolated human organelles, corresponding to the in vivo situation in yeast [6] [23].

The amount of the imported RNA increased upon addition of rabbit enolase to the import mixture in combination with preKARS2, however, the effect of enolase was dependent on the RNA structure. tRK1 was very poorly imported with preKARS2 alone but its import has been significantly improved upon addition of either rabbit or yeast enolase (FIG. 3A), demonstrating the interchangeability of the yeast and mammalian targeting systems. The recombinant human enolase (hEnol) had the same effect on the tRK1 import in vitro as the rabbit one (not shown).

In contrast to the situation with tRK1, the level of mitochondrial import of the FD-L and FD-H RNA molecules was rather high in the presence of preKARS2 alone and has only been slightly improved upon rabbit enolase addition (FIG. 3B). These data are in agreement with our model suggesting that only in the alternative F-conformation tRK1 acquires a high enough affinity to preMsk1p (FIG. 1A), and the RNA-chaperone activity of enolase is necessary for this structural rearrangement [12]. According to this suggestion, the presence of enolase should not be so important for the FD-L and FD-H RNA molecules, since they do not need the structural rearrangements for the interaction with preKARS2 and mitochondrial targeting.

As expected, the truncated RNA molecules HF and HD, which cannot interact with preKARS2, have not been directed into human mitochondria by this protein, independently of the presence of rabbit enolase (FIG. 3C).

### Example 3

### Implication of preKARS2 in the RNA Mitochondrial Import In Vivo

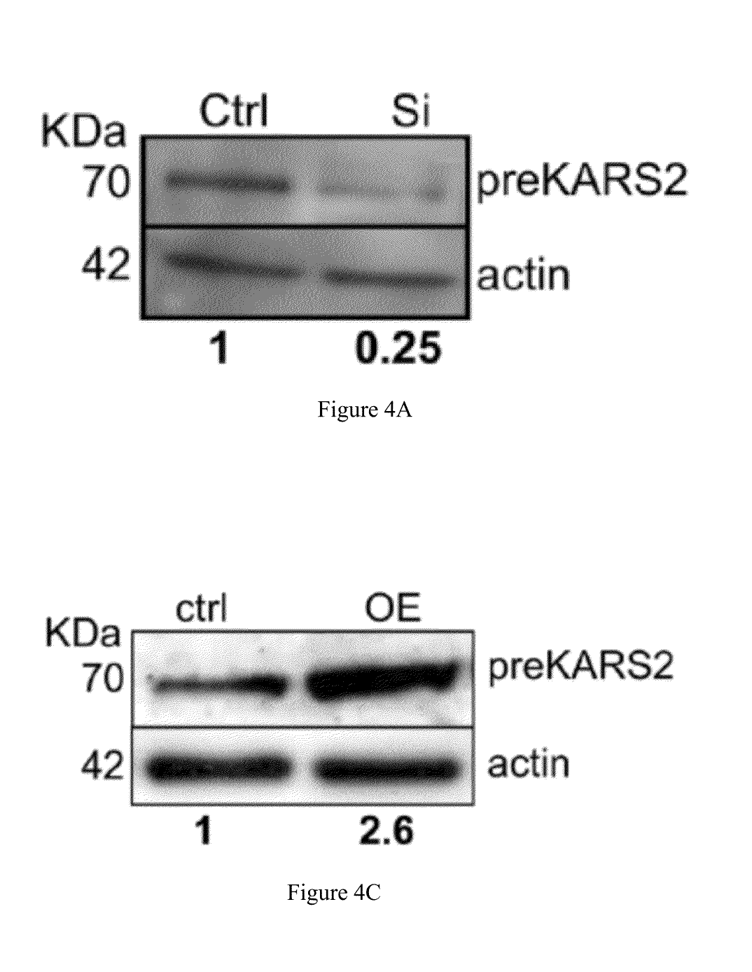

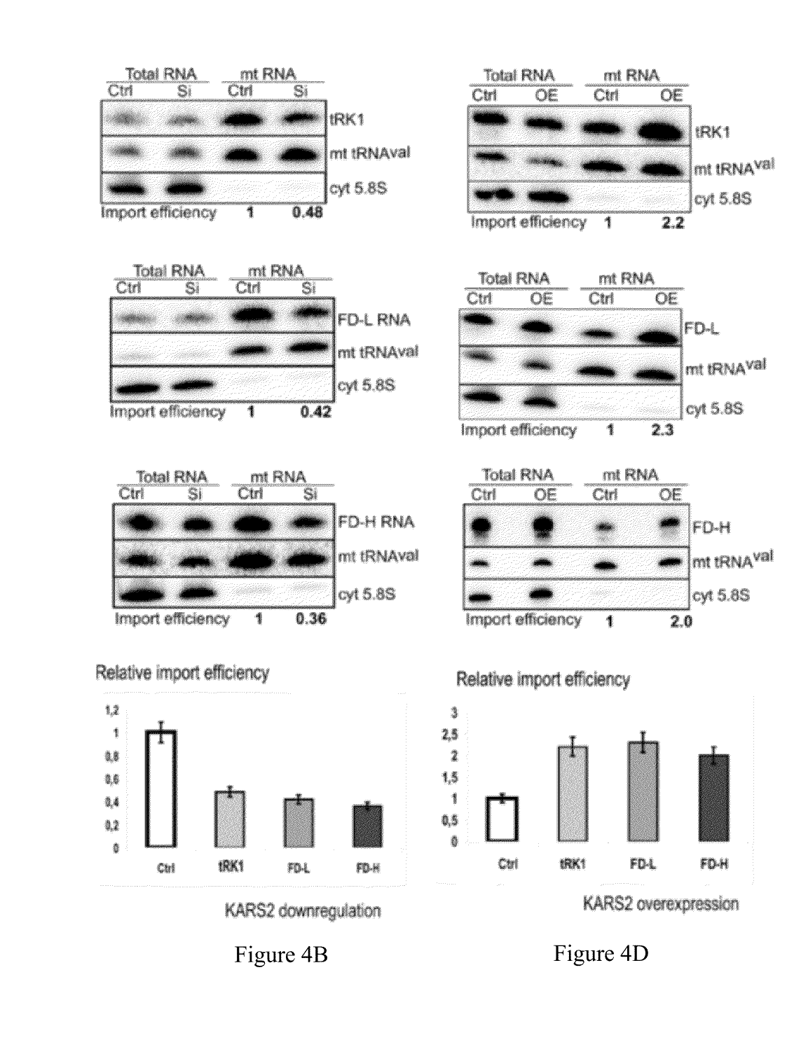

To compare the in vitro and in vivo import requirements, the role of preKARS2 in the mitochondrial RNA targeting was studied in cultured human cells. For this, the inventors used the in vivo import assay on the cells transfected with RNA molecules, as described (9). To downregulate preKARS2, cultured human HepG2 cells were transiently transfected with a mixture of two siRNAs specifically designed against the part of the preKARS2 mRNA corresponding to the mitochondrial targeting sequence. Three days after the second transfection (see Methods section for details), a drop of more than 70% was observed for preKARS2 by Western blot (FIG. 4A). To evaluate the effect of the preKARS2 downregulation on the RNA import into mitochondria, the cells were transfected with purified T7-transcripts of tRK1, FD-L or FD-H. The whole cell RNA and mitochondrial RNA were isolated from the control and preKARS2-downregulated cells and analysed by Northern blot hybridization (FIG. 4B). The absence of signal in the mitochondrial RNA after hybridization with the probe against the cytoplasmic 5.8S rRNA indicates that the treatment of mitochondria with ribonuclease and digitonin removed all contamination by cytoplasmic RNA. The amount of tRK1 molecules internalized by the cells was quantified by Northern blot hybridization using known amounts of T7-transcripts loaded on the same gel as standards. By this approach, it could estimate that 10.860.5% of the tRK1 added to the cells were internalized and could be detected in the full-size form 48 h after transfection. This value corresponds to 2.6±0.2×10⁶ RNA molecules per cell, which number is in the range of most abundant cellular RNAs, for example, 5S rRNA, estimated previously as 3.6±0.5×10⁶ RNA molecules per cell. The number of tRK1 molecules in the mitochondrial fraction corresponded to 4.6±0.4×10⁴ RNA molecules per cell, giving 2.5±0.3% of the molecules imported into mitochondria from the cellular pool, which perfectly correlates with our in vitro data.

Inventors observed a clear difference in the mitochondrial RNA import between the control and preKARS2-downregulated cells: the tRK1 import decreased 2-fold, and a 2.5-3-fold reduction was observed for the small artificial FD-L and FD-H RNAs import (FIG. 4B).

To confirm the role of preKARS2 as a mitochondrial targeting factor for tRK1 and its derivatives, the inventors tested the RNA mitochondrial import in cells overexpressing preKARS2. For this, they used HeLa Tet-Off cells transiently transfected with a plasmid expressing preKARS2 (generous gift of M. Mirande, Gif-sur-Yvette, France). In 48 h after transfection, a 2- to 3-fold increase of the preKARS2 protein amount in the cell extract was detected (FIG. 4C), in agreement with previously published data. The cells overexpressing the preKARS2 protein were transfected with tRK1, FD-L or FD-H, and the mitochondrial RNA import was analysed by Northern blot hybridization (FIG. 4D), compared to control cells transfected with an empty vector. The mitochondrial import of all three RNA molecules, tRK1, FD-L and FD-H, increased 2-fold in the cells over-expressing preKARS2, confirming that the amount of the RNA molecules penetrating into mitochondria in human cells depends on the level of the preKARS2 protein expression.

All the data presented above clearly indicate the role of the human mitochondrial lysyl-tRNA synthetase preKARS2 in the mitochondrial targeting of yeast tRK1 and the artificial RNA molecules containing two structural elements of the tRK1 alternative “import-active” fold, the D-arm and the F-hairpin.

### Example 4

### Mitochondrial Import of Truncated RNA Molecules is not Dependent on preKARS2

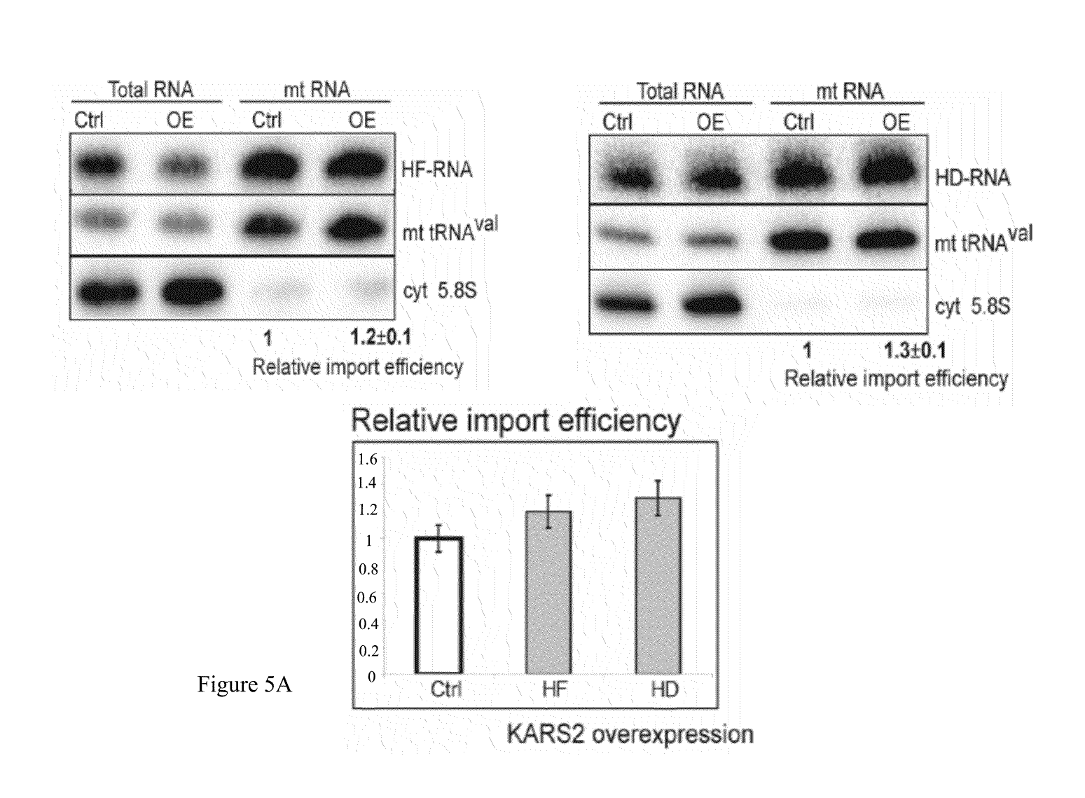

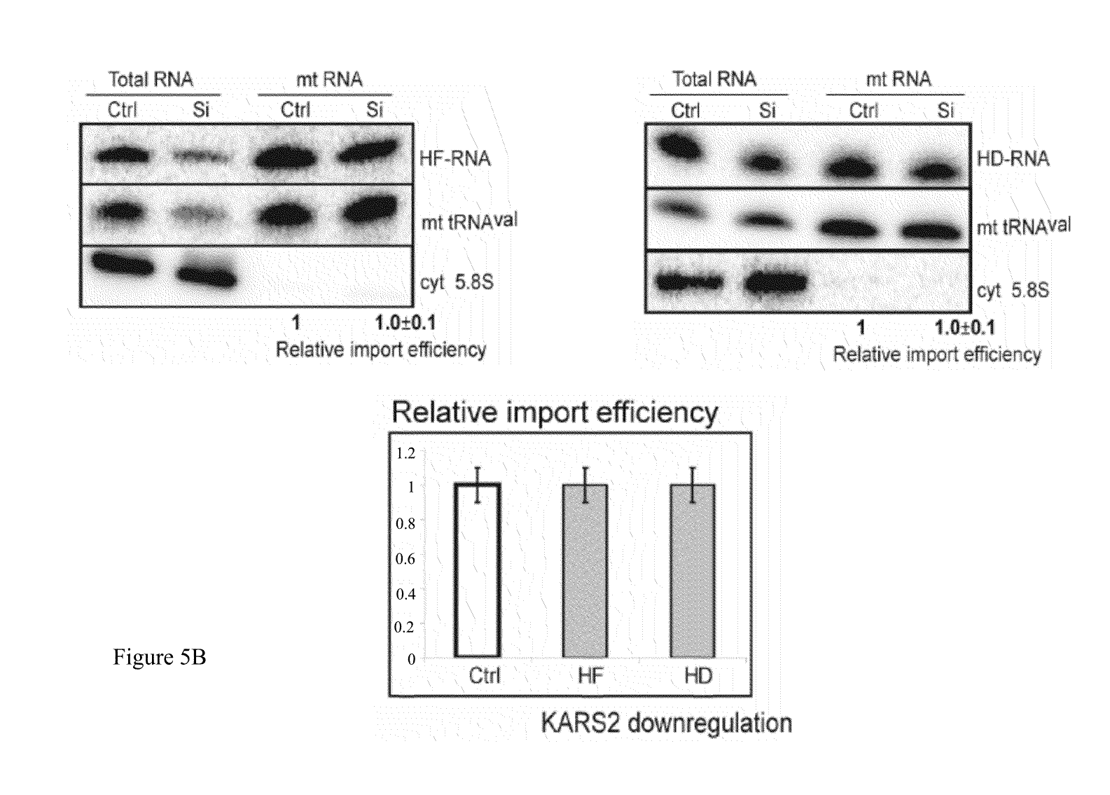

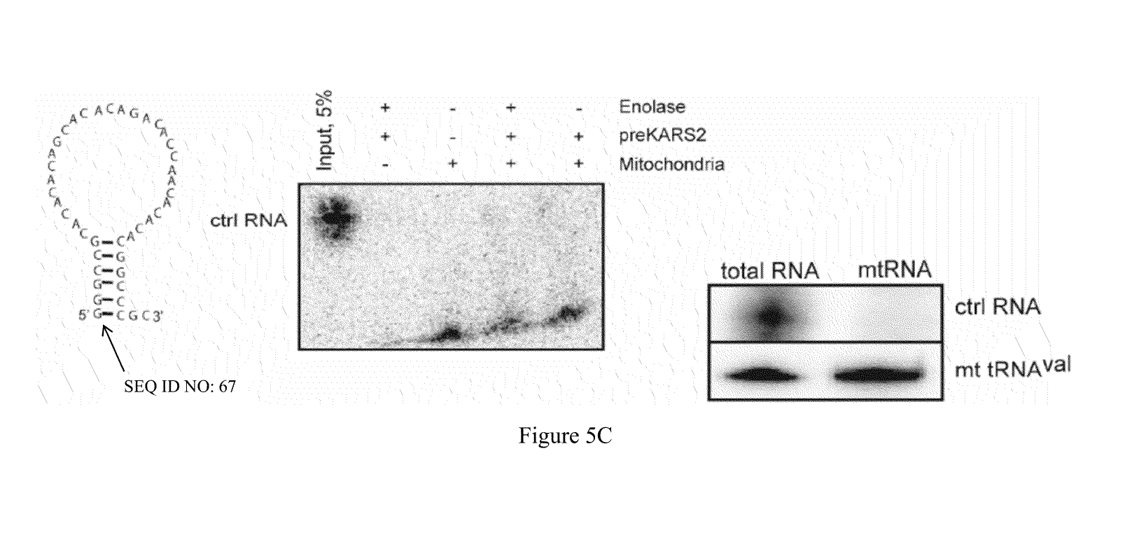

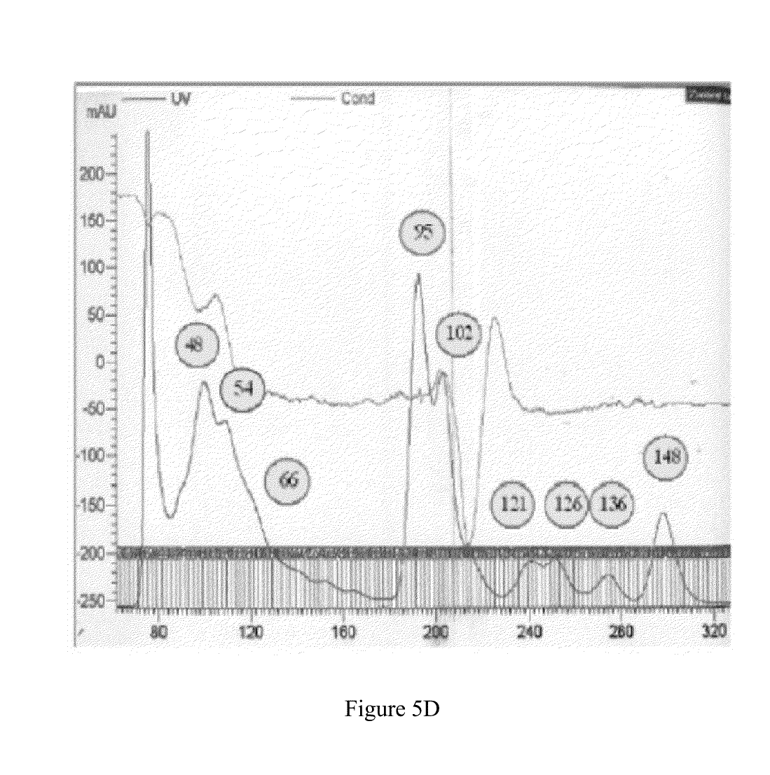

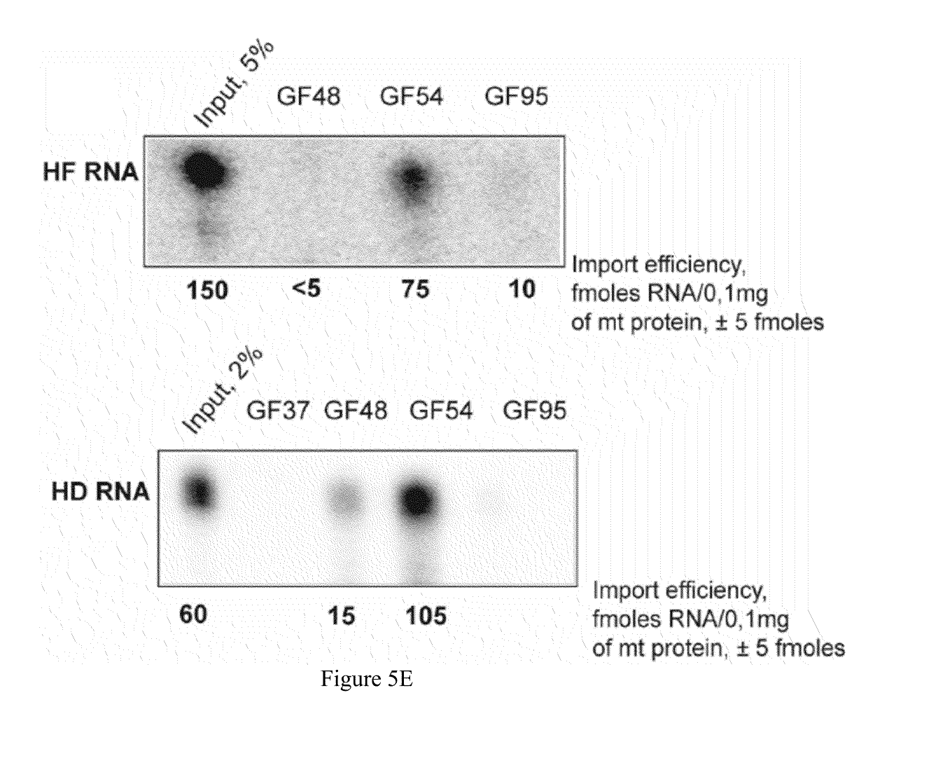

Surprisingly, the small artificial RNA molecules containing either the D-arm or the F-hairpin (referred to as HD and HF, FIG. 1C), which were not imported into isolated human mitochondria in vitro, were internalized by mitochondria in vivo (FIG. 5A, B). A possible explanation of this discrepancy could be that our in vitro import conditions may not allow for a correct (predicted) folding of the short truncated RNA molecules. Nevertheless, the same RNAs internalized by cells were able to be folded and imported into mitochondria.

To verify the specificity of our import test, the inventors designed an artificial control RNA of a size similar to that of the HF and HD molecules (43 nt) but unrelated to yeast tRK1 and containing a short G-C stem and a long unstructured loop (FIG. 5C). This control RNA was not able to interact with the recombinant preKARS2 and be imported into isolated human mitochondria in the presence of the purified proteins, preKARS2 and rabbit enolase (FIG. 5C, middle panel). Contrary to HD and HF RNAs, the control RNA was not detected in mitochondria of transfected HepG2 cells (FIG. 5C, right panel), indicating that not any short RNA molecule can be imported, but only those containing the structural import determinants.

As has been shown above, the HD and HF RNA molecules lack the capacity to interact with the recombinant preKARS2 and be imported into isolated human mitochondria in the presence of preKARS2 and rabbit enolase (FIG. 3C). In agreement with these data, the in vivo import of these RNAs was not dependent on preKARS2, since no change in the amount of the RNA molecules transported into mitochondria was observed when preKARS2 had been transiently downregulated or overexpressed (FIG. 5A, B). This suggests implication of other protein factor(s) in the import of these RNAs into mitochondria in vivo.

To check if the mitochondrial targeting of the truncated RNAs is still dependent on protein factors, inventors isolated crude proteins from HepG2 cells, fractionated them by gel-filtration and tested the main peaks, each representing a mixture of many proteins, for their ability to direct RNA into isolated human mitochondria (FIG. 5D). The inventors detected an efficient import of both truncated RNAs in the presence of one protein fraction (FIG. 5E), thus demonstrating that the in vitro mitochondrial import of the HD and HF RNA molecules is dependent on protein factors. All presented data show that RNA targeting into human mitochondria is a flexible process, allowing to import not only a full-size yeast tRNA but also its truncated versions. Import of tRK1 and the RNAs containing both tRK1 import determinants depends on the preKARS2 protein. Shorter truncated molecules were shown to be imported with the help of other, so far unidentified protein factor(s).

Conclusion 1: Induced tRNA Import into Human Mitochondria

Implication of a Host Aminoacyl-tRNA-Synthetase

PreKARS2 as a tRK1 Carrier to Human Mitochondria

In human cells, a subset of small non-coding RNA is imported into mitochondria from the cytosol[26], including some tRNAs (either in a natural or an artificial manner), the RNA components of RNase P and MRP endonuclease, and 5S rRNA. Analysis of the cryptic tRNA import pathway, allowing the targeting of the yeast tRNALys CUU into human mitochondria, performed in the present study demonstrated a similarity between the tRK1 import mechanisms in yeast and human cells. In yeast cells, preMSKlp and Eno2p were identified as the tRK1 mitochondrial targeting factors. A similar tRNA import pathway in human cells involves the orthologous proteins, preKARS2 and enolase. Moreover, the alternative folding of tRK1 as a determinant for the mitochondrial targeting in yeast (9) seems to be relevant in human cells as well, since the inventors show that artificial RNA molecules containing two hairpin structures characteristic for the tRK1 alternative F-fold (FIG. 1A) can be efficiently imported into human mitochondria in vitro and in vivo, in a manner clearly dependent on the preKARS2 protein (FIG. 3, 4).

Aminoacyl-tRNA-synthetases are a group of enzymes responsible for the specific attachment of amino acids to their cognate tRNAs, thus performing a key step of translation. In human cells, one gene KARS1 codes for both mitochondrial and cytosolic lysyl-tRNA-synthetases which are produced from two mRNAs generated by alternative splicing. PreKARS2 possesses a specific N-terminal sequence of 49 amino acid residues, which is the only difference from KARS1. The situation is the opposite in yeast S. cerevisiae where the mitochondrial and cytosolic lysyl-tRNA-synthetases are encoded by distinct genes, MSK1 and KRS1. PreMSKlp plays an essential role in the mitochondrial targeting of the cytosolic tRNA^{Lys} CUU (tRK1) in yeast.

Previously, it has been demonstrated that human preKARS2 overexpressed in yeast can partially complement the growth defect associated with the loss of MSK1 and can additionally facilitate the import of tRK1 into isolated yeast mitochondria [21]. Here the inventors demonstrate the direct interaction of preKARS2 (but not of its mature form) with yeast tRK1 and the involvement of this protein in the tRK1 import into human mitochondria in vivo.

Recently and rather surprisingly, the mature mitochondrial enzyme KARS2 was shown to interact with the human cytosolic tRNALys3 with an apparent Kd of 250+/240 nM, but the presence of the mitochondrial targeting sequence in preKARS2 completely abolished the RNA-binding properties of the protein (Kd 0.1 mM for preKARS2). Since in human cells no import of tRNA^{Lys} into mitochondria had been observed, the apparent discrepancy between these and our data clearly indicates a different mode of preKARS2 interaction with either the nonimportable cytosolic tRNA^{Lys} or the importable tRK1. This is in agreement with our hypothesis that only the alternative fold of tRNA can be recognized by the precursor of mitochondrial lysyltRNA-synthetase functioning as an RNA mitochondrial carrier. Thus, only yeast tRK1 and some specially designed RNA molecules capable to adopt the alternative conformation can interact with preKARS2 and be targeted into human mitochondria.

RNA Targeting into Mitochondria: A Species-Specific or a Universal Mechanism?

In general, each known case of RNA mitochondrial import appears somewhat special and thus not sufficient to establish a common RNA import mechanism. The results of the present work, together with our previous data, enable us to revisit the paradigm of ‘extremely diversified’ RNA import pathways and to propose several rules which can be, if not universal, at least largely applicable to various RNA import systems.

Firstly, to be imported into mitochondria, an RNA should escape from the cytosolic channelling. According to this model, no free diffusion of macromolecules inside the cell is normally possible since all its components are well arranged in space and their movements are strictly regularized (channelled). Channelling was studied in detail on the example of tRNAs. It was found that, starting from the very transcription event, a tRNA molecule is trapped in a standard sequence of events (processing, modification, nuclear export, translation) assured by protein components that function in a chain. They hand the tRNA from one to another avoiding its release into solution. To make an RNA exit from the standard circuit, a well regulated deviation has to be provided by a special mitochondrial targeting factor which has a specific affinity to the cargo RNA. For example, in yeast cells, tRK1 is probably captured from the translation cycle by the glycolytic enzyme enolase and redirected to the mitochondrial surface. The same event apparently exists in the artificial tRK1 import pathway in human cells. In the case of the 5S rRNA import, this function is performed by the cytosolic precursor of mitochondrial ribosomal protein L18 (preMRP-L18). To assure the irreversible RNA withdrawing from the cytosolic channelling, the protein factor should possess a chaperone activity to change the RNA conformation, as has been shown for tRK1 in the complex with yeast enolase or for 5S rRNA and preMRP-L18. The next step of the pathway is a rapid discharge of the chaperone by another mitochondrial import factor. Examples of such a cascade were described in the yeast import mechanism where tRK1 is quickly transferred from enolase to the precursor of lysyl-tRNA synthetase. A very similar case was observed for 5S rRNA in human cells where the mitochondrial enzyme rhodanese accepts 5S rRNA from preMRP-L18. For both mechanisms, a significant decrease in the apparent dissociation constant for the complex between the second protein factor and the RNA was found. Then, the second import factor works as a carrier transporting the RNA molecule into the mitochondria. The mechanism of RNA translocation across the double mitochondrial membranes is not yet understood. Most probably, it exploits the standard mitochondrial pre-protein localisation apparatus, since carriers usually have signals of mitochondrial localisation and it appears the most obvious way to reach the organelles. Nevertheless, one cannot exclude alternative translocation mechanisms via different membrane channels.

Thus, for all RNA import systems in which the pre-mitochondrial (targeting) step of RNA import has been investigated, several universally present features can be outlined. Namely, in order to direct a cytosolic RNA to mitochondria one needs necessarily two protein factors, the first with a chaperone activity to withdraw the RNA from the cytosolic channeling, the second possessing the signal of mitochondrial localisation to target the RNA into the mitochondria. One of these proteins should be cognate, interacting with the imported RNA in a specific way and thus determining the selectivity of the RNA import (preLysRS for tRK1, preMRP-L18 for 5S rRNA). The other protein factor may be unrelated to RNA metabolism and hardly expected to participate in RNA transport, performing thereafter a “second job”, as enolase and rhodanese. Concerning enolase, many non-glycolytic “moonlighting” functions of this protein are known. In E. coli, enolase is an integral component of the RNA degradosome; in yeast, it was identified as Hsp48 and participates in formation of vacuoles; enolase is found in the eye lens of many organisms and as a plasminogen-binding receptor expressed on the surface of a variety of eukaryotic cells. Thus, the tRNA import into mitochondria seems to be one of many different functions of this enzyme.

The common rules described herein can be applied in the search for RNA import pathways in various eukaryotes. For instance, it appears that in plant cells, precursors of dually targeted cognate aminoacyl-tRNA synthetases combine both RNA targeting functions and thus may be the only essential tRNA import factors. Probably, the same situation may be found in Trypanosoma brucei, where the cytosolic elongation factor eEF1a assures the specific targeting of almost all tRNAs to mitochondria.

Mitochondrial Import of Small RNA Molecules

The general rules of RNA import formulated above presume a certain flexibility of the pathway. Indeed, various RNA molecules able to interact with import factors can be targeted into mitochondria even in organisms naturally importing only a very restricted number of RNA species, as the inventors see here for the RNAs FD-H and FD-L. On the other hand, various proteins might perform the function of RNA import factors in certain conditions.

This possibility is clearly demonstrated in the present work since the short truncated RNA molecules HD and HF, which have lost the capacity to interact with preKARS2, apparently can be targeted into human mitochondria with the help of other, still unidentified protein(s). This hypothesis is also in agreement with a recent publication claiming that preMSKlp may be dispensable for the tRK1 import into yeast mitochondria. One can suggest that in the yeast strain used in this study, lacking the MSK1 gene and thus devoid of actively respiring mitochondria, the small amount of tRK1 detected in the pro-mitochondria could be imported by a backup pathway with a help of alternative targeting protein(s).

Recently, a subset of microRNAs, small non-coding RNAs that associate with Argonaute proteins to regulate gene expression at the post-transcriptional level, has been localized to human mitochondria as well as the AGO2 protein [45]. At least a part of these miRNAs and their precursors were supposed to be imported from the cytoplasm by an unknown mechanism. It would be tempting to hypothesize that small structured RNA molecules, such as HD and HF, might be recognized by the machinery of the miRNA import and targeted to mitochondria by AGO2 and/or another component of the RNA-inducible silencing complex (RISC), which could thus perform the “second job” as mitochondrial targeting factors, similarly to the enzymes enolase or rhodanese. This exiting possibility remains to be explored in future studies.

### Characterization of Chemically Modified Oligonucleotides Targeting a Pathogenic Mutation in Human Mitochondrial DNA

### Example 5

### Stability of Modified Oligonucleotides in Human Cells

Chemical substitutions at the 2′-hydroxyl group with 2′-OMe, 2′-F and 2′-deoxy in combination with terminus capping chemistry often improve the stability of therapeutic oligonucleotides. To test this methodology, the inventors used a small artificial RNA D22L (FIG. 6), which was previously shown to be imported into human mitochondria. This molecule represents the fusion of the D-arm of imported tRNALys (tRK1) (9) with a 22-nucleotide sequence corresponding to a fragment of human mtDNA at the boundaries of a large deletion found in patients with Kearns Sayre Syndrome (KSS). Since this anti-genomic part of the molecule (referred to as KSSpart) should be the most sensitive to nucleases, a set of D22L derivatives containing substitutions at the 2′-hydroxyl group with or without addition of inverted 3′-3′ thymidine nucleotide at the 3′ terminus (FIG. 6) was synthesized. All the pyrimidine nucleotides of the KSS-part contained 2′-OMe (FIG. 6, green pyrimidines) and 2′—F groups (FIG. 6, blue pyrimidines) in the versions D22L-Me and D22L-F correspondingly. In the D22L-4Me molecule, only four nucleotides localized in the nuclease-sensitive sites contained the 2′-OMe groups. In D22L-DNA molecules, all the nucleotides of KSS-part were in 2′-deoxy form, thus forming a set of chimerical RNA-DNA molecules. Inventors also synthesized and tested chimerical RNA-DNA molecules containing RNA F-hairpin structure as the mitochondrial import determinant and DNA KSS part of 13 nucleotides complementary to either H- or L-strand of mtDNA (F13H and F13L correspondingly).

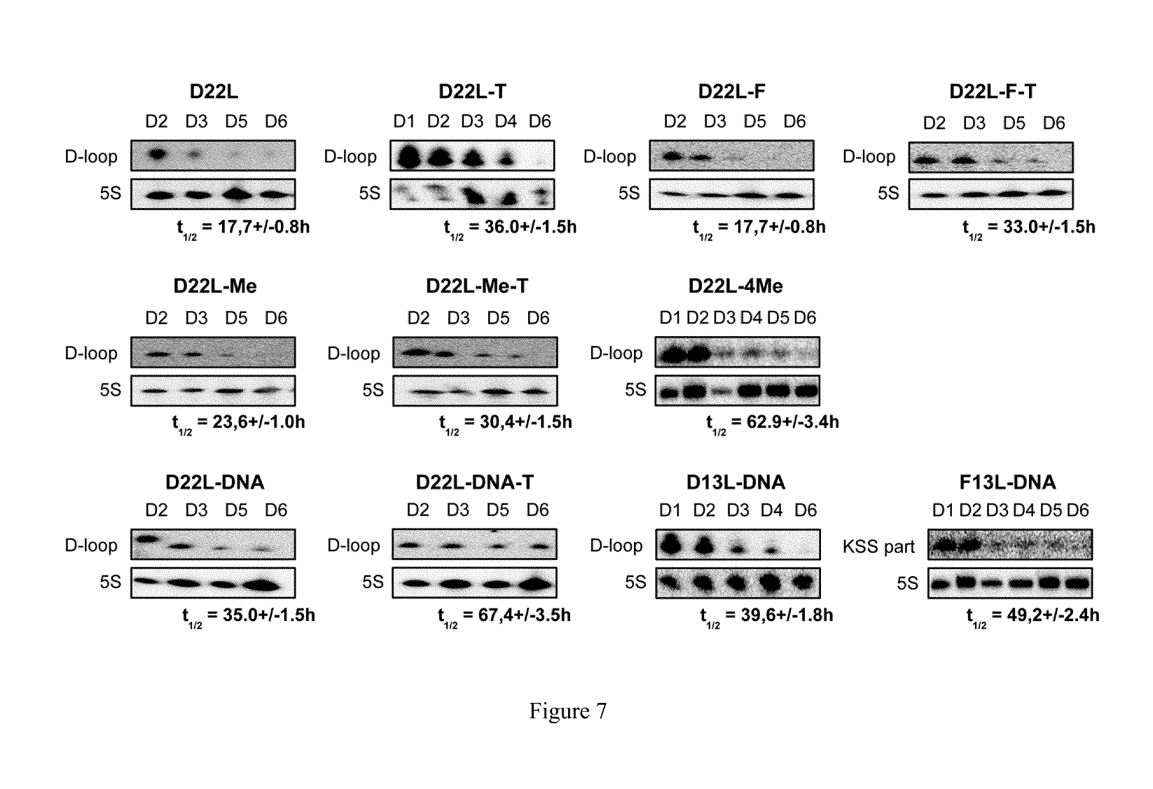

All versions were tested for their stability in human cells. For this, cultured cybrid cells were transfected with synthetic oligonucleotides (referred to as SO). The transfection procedure did not lead to a detectable decrease of viability of the cells, suggesting the absence of SO toxicity. The amounts of SO internalized by cells and their degradation rates were evaluated by Northern hybridization of total cellular RNA with specific probes at different time points spanning a 6-day period after transfection (FIG. 7). The nonmodified RNA molecule D22L demonstrated a rather low stability in cells, characterized by a half-life period of 17.7+/−0.8 h. On the other hand, protection of its 3′-terminus by inverted 3′-3′ thymidine resulted in an increase of stability to 36.0+/−5 h. Surprisingly, 20-modifications of all the pyrimidine nucleotides in D22LMe and D22L-F molecules did not lead to further increase of their stability comparing to D22L, while 20-O-methylation in only four positions, representing the U-A, C-A and U-G sites, previously identified as major cleavage sites for serum endoribonucleases, resulted in a strongly increased lifetime of the D22L-4Me molecule. A strong effect on SO stability was also detected for molecules containing a DNA KSS-part. For these chimerical SO, increased stability was detected if the 3′ terminus had been protected by RNA hairpin structure in F13L-DNA or by inverted 3′-3′ thymidine in D22L-DNA-T (FIGS. 6 and 7).

The above data indicate that an increase of anti-replicative molecules lifetime in transfected cells can be achieved either by the use of RNA-DNA chimerical versions and RNA molecules bearing 2′-OMe groups in nuclease-sensitive sites.

### Example 6

### Mitochondrial Import of SO and mtDNA Heteroplasmy Analysis

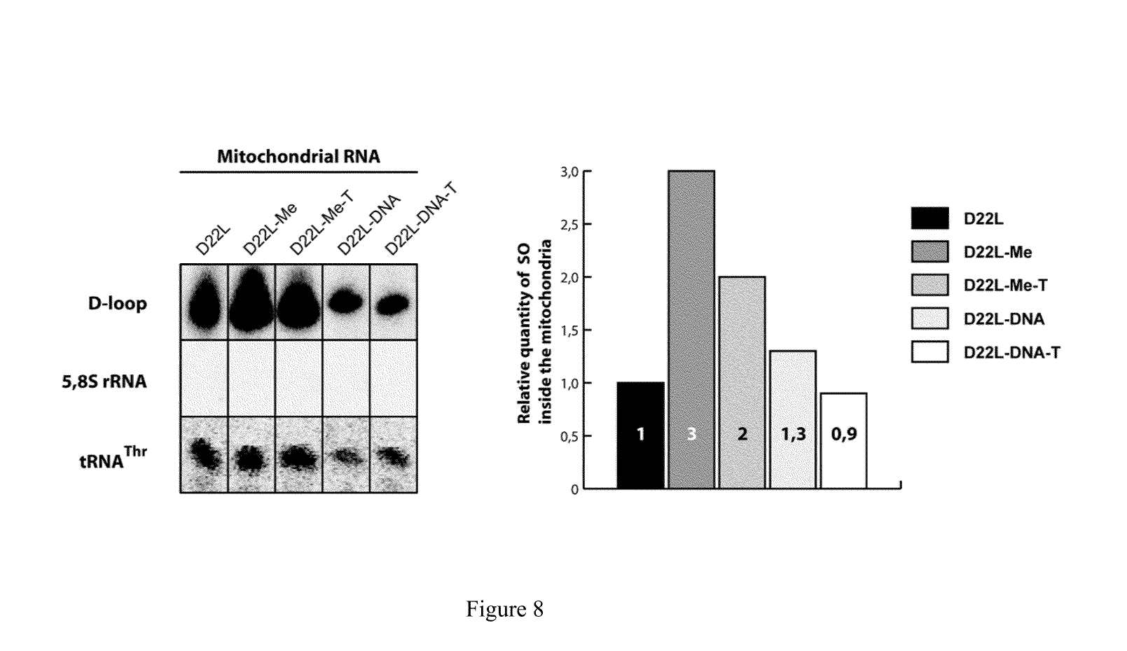

To determine the impact of modified nucleotides on the mitochondrial import of SOs, the amounts of D22, D22-Me, D22-Me-T, D22-DNA and D22-DNA-T in purified RNasetreated mitochondria 2 days after transfection (Figure. 8) were compared. Hybridization data using SO-specific probes were quantified and normalized to the amounts of loaded mtRNA, estimated by hybridization with a probe to a mitochondrial transcript (mt tRNAThr). The results demonstrate that chemical modifications do not inhibit the mitochondrial import of the molecules. Moreover, for the versions contained 2′-OMe groups, an increased efficiency of import in comparison with the non-modified RNA D22L was detected. Amounts of chimerical molecules inside mitochondria were comparable to those of non-modified RNA D22L. Therefore, the presence of inverted thymidine at the 3′ terminus and modifications of ribose in KSS-part of SO had no negative effect on the mitochondrial uptake of these molecules, probably indicating a correct interaction of the modified SO with protein import factors and mitochondrial membrane receptors and channels.

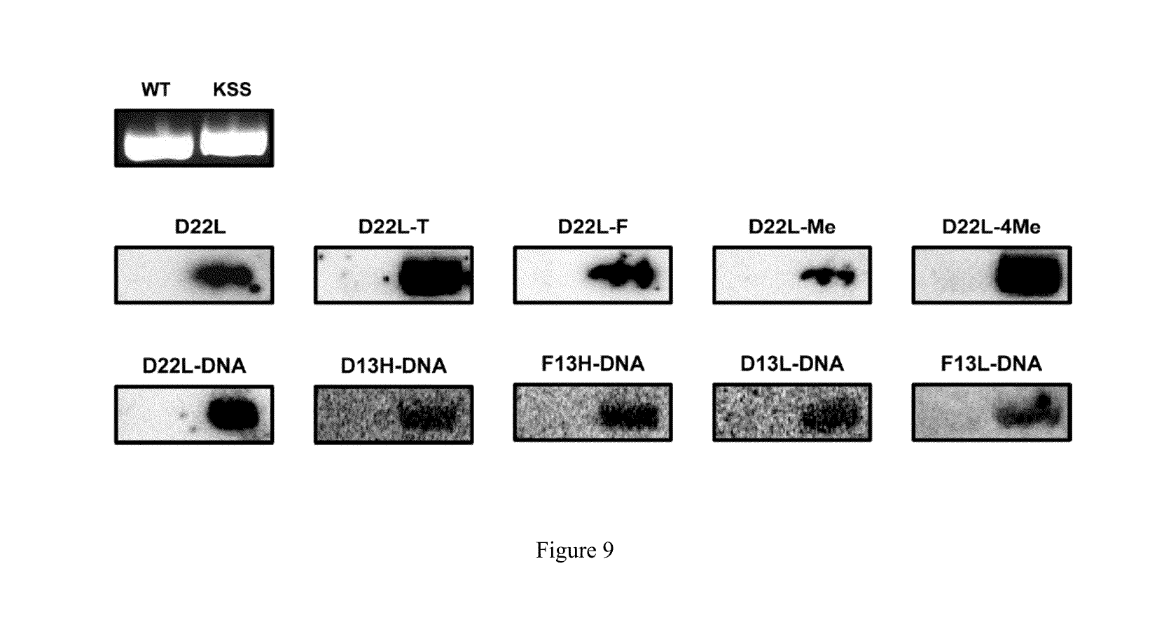

To induce a shift of the mutant mtDNA proportion, the therapeutic anti-replicative molecules should anneal in a very specific manner to mutant mitochondrial genomes, but not to wild-type ones. Predicted and experimentally measured melting temperature values for the hybrids between the SO and mutant KSS mtDNA show the increased stability of the duplex for chimerical and 2′-O modified oligonucleotides compared to D22 (Table 1). To assay the specificity of modified SO annealing to mtDNA, Inventorsperformed Southern blot-hybridization of labelled SO with fragments of mutant and wild-type mtDNA at 37° C. in a buffer containing physiological concentrations of NaCl (150 mM) (FIG. 9). The results clearly demonstrate that all the SO analysed could hybridize only with mtDNA fragment containing the deletion boundaries, but not with the wild-type mtDNA fragments.

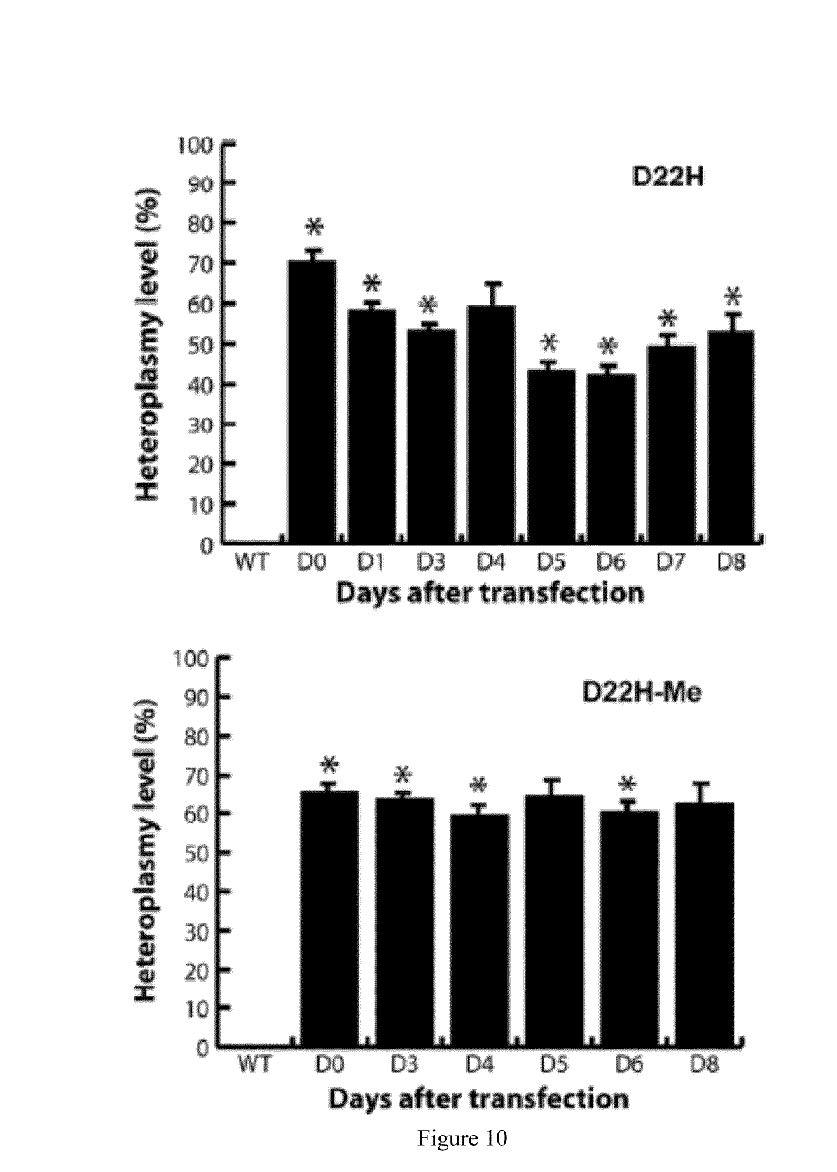

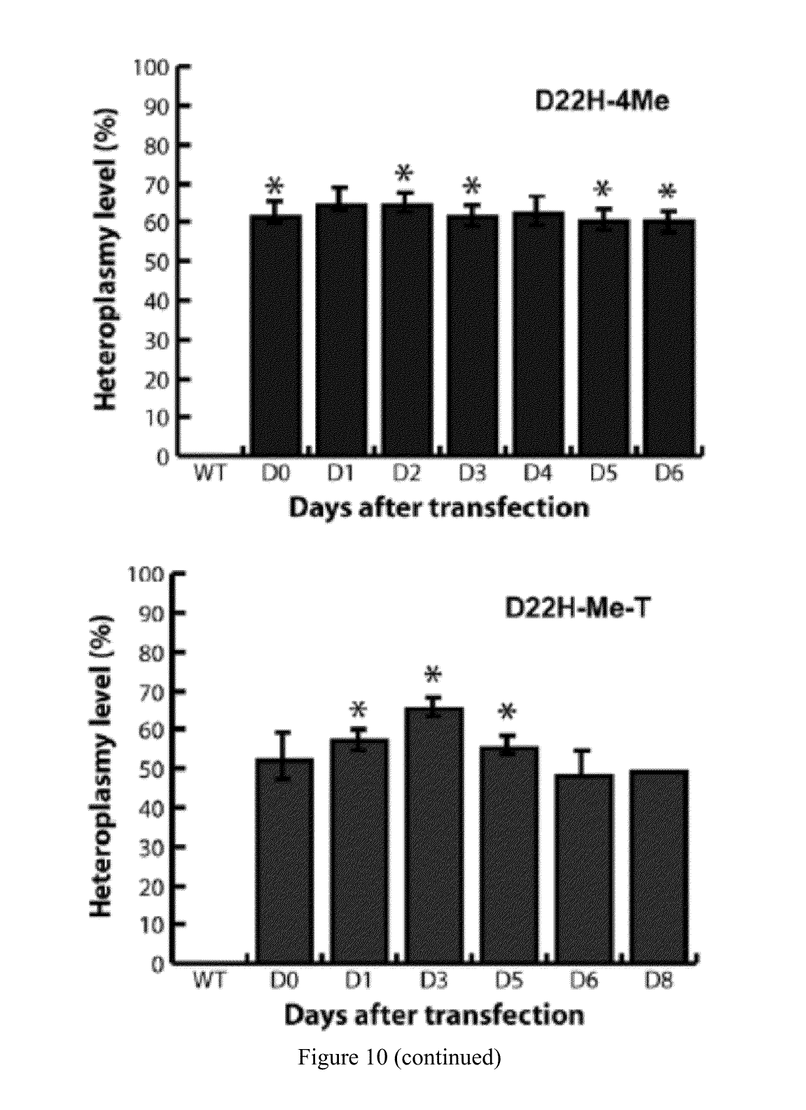

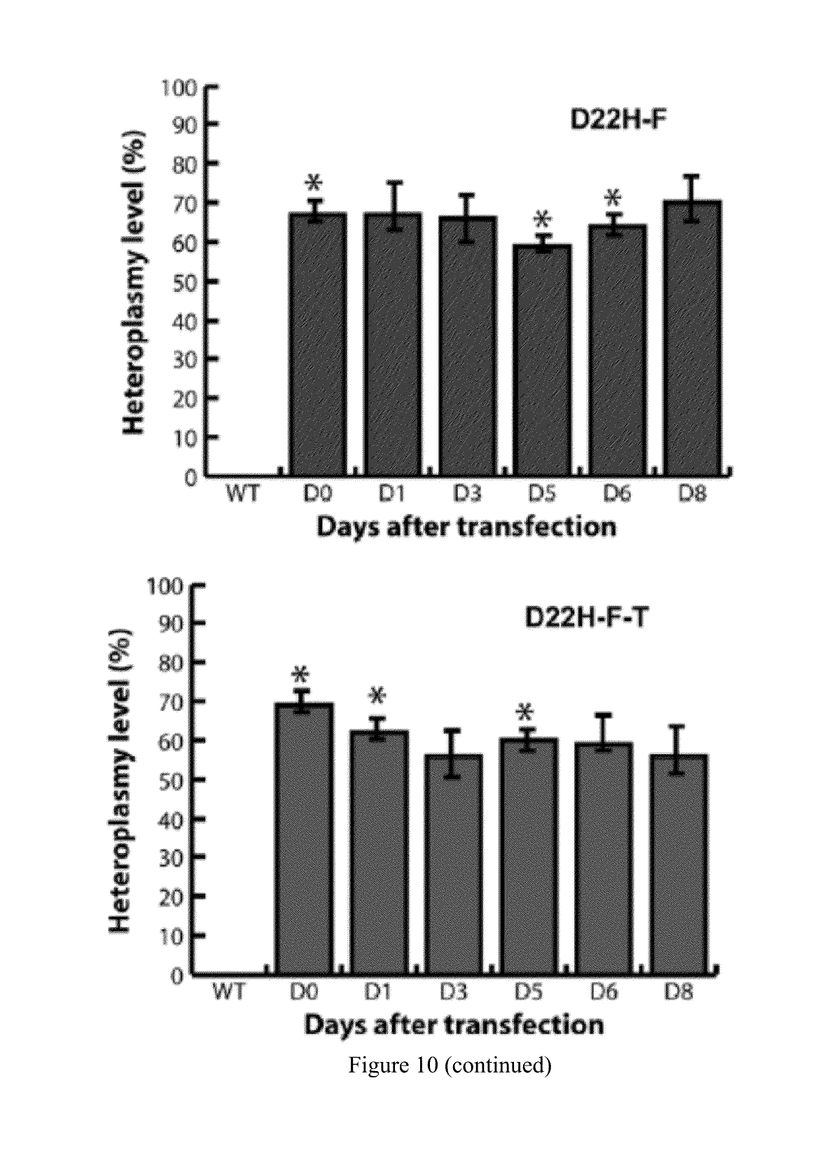

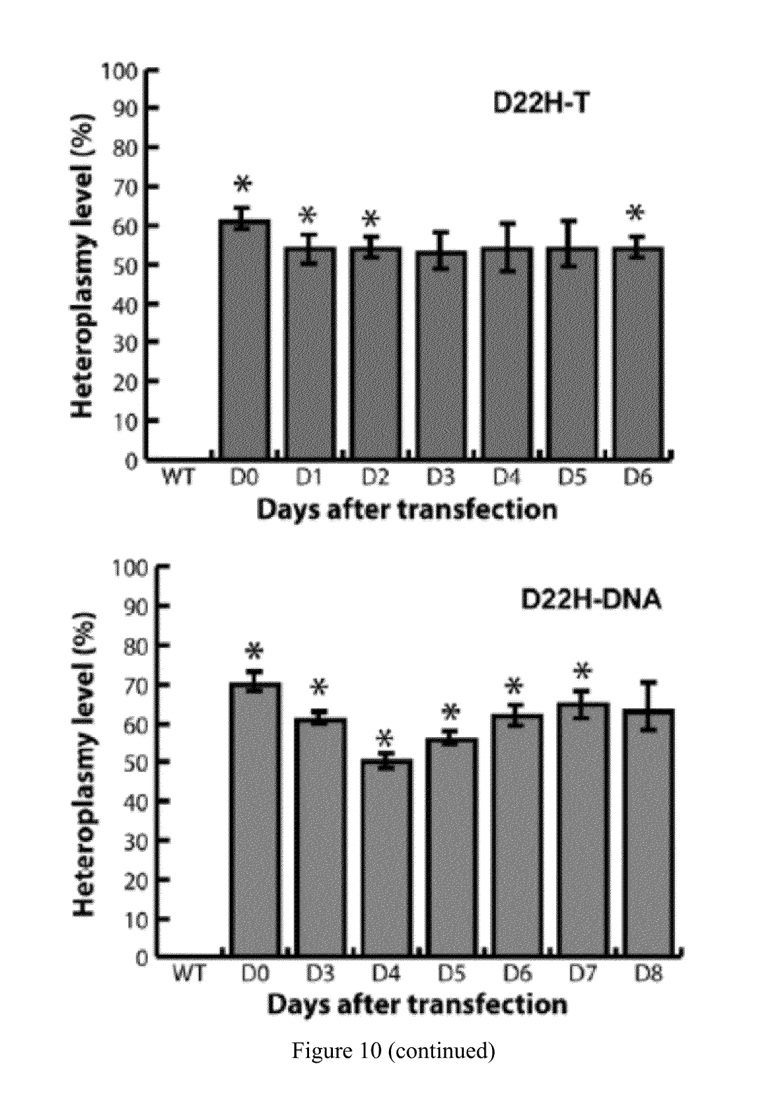

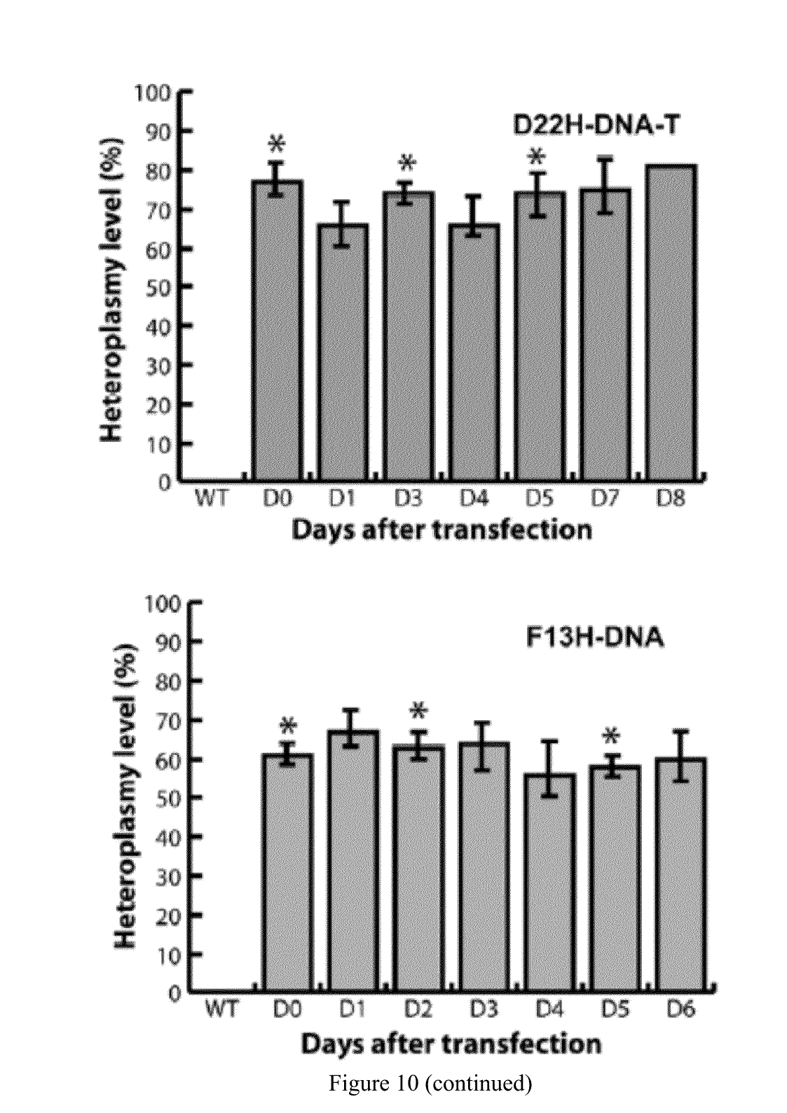

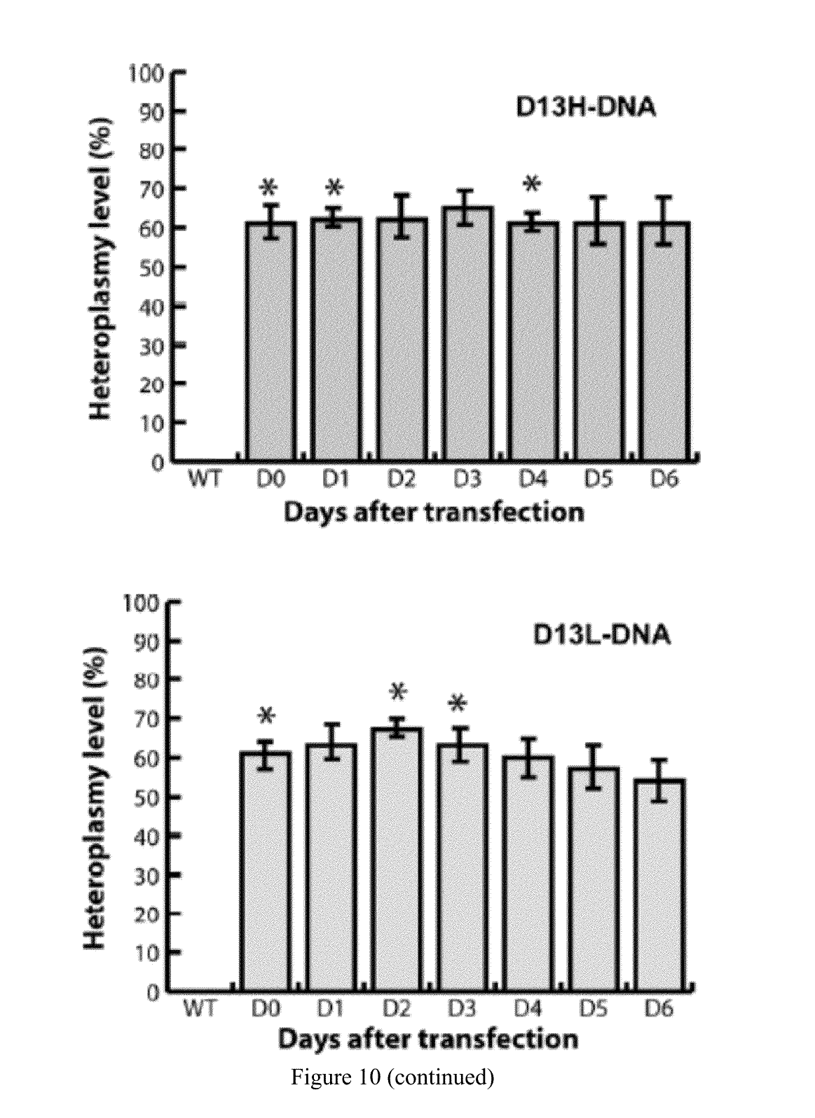

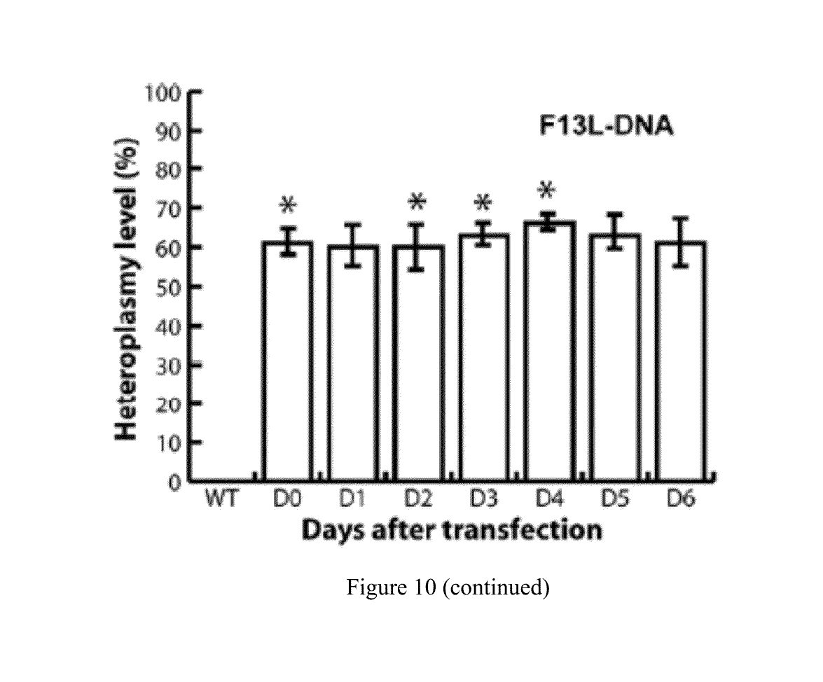

Modified SOs are thus capable of being targeted into human mitochondria in living cells and to anneal with the mutant form of mtDNA, so the inventors anticipated to detect their effect on the KSS deletion heteroplasmy level. They therefore measured the percentage of mutant KSS mtDNA to all mtDNA molecules by real-time qPCR in cybrid cells transfected with various SO at different time points spanning a 6-8 day period after transfection (FIG. 10). Contrary to our expectations, only non-modified RNA D22H induced a reproducible decrease of heteroplasmy level by 20+/−2% with a 5-6 day delay in respect to the cell transfection. The effect had a temporal character, since the proportion of mutant mtDNA reincreased within 7-8 days after transfection, as it was previously shown for molecules containing two import determinants. Among all the modified SO tested, only chimerical molecule D22-DNA induced a 15% shift of the heteroplasmy in 4 days after cell transfection. The other versions did not cause any significant effect on the heteroplasmy level. Therefore, the modifications of 2′-OH ribose group, as well as inverted thymidine residue added to the 3′ terminus, had a negative effect on the anti-replicative abilities of the SO, most probably due to the inability of the modified molecules to form correct and stable complex with mutant mtDNA in vivo.

Conclusion 2: Characterization of Chemically Modified Oligonucleotides Targeting a Pathogenic Mutation in Human Mitochondrial DNA

Stability of the Modified Oligonucleotides in Human Cells

Serum and cell homogenates are known to contain ribonucleases that belong to the RNase A-, 3′-exonuclease-, and phosphatase families, which cleave RNA more readily at sites within single strand regions and loops. Modifications at the 2′ position of the sugar ring, including 2′-OMe and 2′-F, confer to the oligonucleotide the capacity to adopt an RNA-like C3′-endo (N-type) sugar pucker, which is the most energy-favourable conformation of RNA. Thus, such modifications increase Watson-Crick binding affinity and, due to the proximity of the 2′-substituent and the 3′-phosphate, improve nuclease resistance. In full agreement with the expectation, the degradation rate of chimeric RNAeDNA molecules in human cells was much lower than that of non-modified RNA D22L. Protection of the 3′ terminus by 3′-3′ inverted thymidine was also very efficient for all the versions, indicating that 3′-exonucleolitic degradation has a very important impact on the SO decay in cells.

Surprisingly, it detected only a very slight increase in stability for the molecule D22-Me, in which all the pyrimidine residues bore 2′-OMe groups, and no effect of 2′—F groups in D22-F version. At the same time, methylation of four predicted nuclease-sensitive sites resulted in a very important increase of a half-life time of the D22-4Me molecule. To explain these data, one can hypothesize that modification of the 16 pyrimidine nucleotides of the 22-nucleotide anti-mtDNA part of the molecule might induce substantial changes in the geometry of RNA molecule, the instability of the D-helix and, therefore, the vulnerability of the 5′-part of the molecule. Noteworthy, modification of only 4 nucleotides did not disturb the structure of the D-arm, thus improving the stability of the whole molecule.

Mitochondrial Import of Modified Oligonucleotides:

To target SO molecules into mitochondria, inventors used the native RNA mitochondrial import, a pathway that appears to be the unique natural mechanism of nucleic acids targeting into these organelles. While protein mitochondrial import mechanisms are well studied, RNA trafficking into mitochondria is not totally understood due to the complexity of its mechanism and the variability between species. This pathway has been detected in phylogenetic groups as diverse as protozoan, plants, fungi and animals. Several teams studied RNA structural determinants necessary for mitochondrial import and their implications for therapeutic purposes. Studies of tRNA import into mitochondria of Leishmania tropica resulted in a model of RNA targeting into mammalian mitochondria by use of the tag sequence comprising the 23-nucleotide D-arm of tRNATyr and a protein complex RIC, detected only in this particular protozoan organism. Another team proposed to use the 2′-nucleotide stem-loop sequence of the RNase P RNA component (H1 RNA) as a signal, that enables longer fusion RNAs to be imported into human mitochondria.

Inventors have developed several successful therapeutic cellular models based on import determinants of a yeast tRNA, tRK1. Inventors demonstrated that anti-replicative ribonucleotides flanked by only one stem-loop structure, D or F, can be efficiently imported into human mitochondria in vitro and in vivo. Furthermore, inventors show that not only RNA, but also DNA oligonucleotides as well as sequences contained modified nucleotides can be efficiently imported into mitochondria of living human cells if flanked by an RNA stem-loop structure. It can hypothesize that such small hairpin RNA domains might be recognized by mitochondrial membrane proteins allowing subsequent translocation of RNA molecules into the organelles. Mammalian polynucleotide phosphorylase (PNPase), an enzyme at least partially localized in the mitochondrial intermembrane space, may function as such an RNA receptor to recognize these import signals and to allow the translocation of correctly tagged “importable” RNA molecules.

The Low Anti-Replicative Capacity of Modified Oligonucleotides

The data show that all of the ribose modifications used in the study, as well as inverted thymidine residue added to the 3′ terminus, led to a loss of the anti-replicative activity of the oligonucleotides. This may be explained in several ways. First, the increased stability of the RNA-DNA duplex might have a negative effect. To check this possibility, a set of RNA-DNA chimerical molecules with a shorter, 13-nucleotide KSS-part (D13 and F13 versions, FIG. 6) was synthesized. The choice of the length was driven by melting temperature prediction (Table 1), aiming to adjust the Tm of the chimerical molecules to those of non-modified RNA D22L. Contrary to D22L and D22L-DNA, the chimerical molecules of the D13 and F13 series had no effect on the heteroplasmy level, indicating that there is no direct correlation between the melting temperature of the duplex of SO with mutant mtDNA and the anti-replicative effect of SO.

The second possible explanation consists of the non-specific annealing of SO not only to mutant mitochondrial genomes, but also to wild-type ones. Even if in vitro hybridization of all the modified SO with mutant mtDNA fragment was very specific, the situation in vivo might be different due to specific ionic conditions in the mitochondrial matrix and the implication of proteins. So far, the version D22-DNA, characterized by a high Tm of duplex with mutant mtDNA and also by rather high (>37) Tm values for the 5′ and 3′ boundaries of KSS deletion (Table 1), was the only one among the modified SO that demonstrated the expected effect on the heteroplasmy level. This indicates that the predicted annealing with wild-type mtDNA does not correlate with the absence of the specific stalling of mutant mtDNA replication.

All these considerations led us to a conclusion that the most important feature could be the nature of the anti-replicative nucleic acid. Apparently, only oligoribonucleotide stretches complementary to the mutated region of mtDNA can significantly influence its replication by stalling the replisome progression. Deoxyoligonucleotides are much less efficient, probably due to the ability of the mitochondrial replisome helicase to denaturate DNA-DNA hybrids, but not the regions of short RNA-DNA duplexes [30]. All the SO versions bearing ribose 2′-OH modifications and inverted thymidine residues revealed the total loss of anti-replicative efficiency. One can hypothesize that the C3′-endo sugar conformation and 3′-3′ inverted nucleotides might be recognized by the replisome or by other mitochondrial nucleoid proteins as non-natural and quickly eliminated. The RITOLS model of mtDNA replication suggests the presence of displaced H-strands not in the single stranded form, but essentially in the form of RNA-DNA hybrid. Therefore, short RNA-DNA duplexes will not be eliminated by mitochondrial proteins and could cause a stall of the mutant mtDNA replication, leading to a shift in proportion between mutant and wild-type mitochondrial genomes.

The data presented here show that various nucleotide modifications protect RNA molecules introduced into human cells against nucleolitic degradation. Modified oligonucleotides fused to import determinants can be imported into mitochondria in living cells and can specifically anneal with mutant mtDNA. However, despite their increased stability, such modified oligonucleotides are not likely to be used as anti-replicative therapeutic agents. Thus, the problem of the transient effect of short RNA molecules on the heteroplasmy level should be resolved by other means. For instance, a stable expression of specific RNA molecules in the nucleus, or the use of a non-toxic transfection procedure, allowing several consecutive transfections of human cells with therapeutic RNA, may prove promising for the future progress of RNA-based approaches of the therapy of mitochondrial diseases.

### Modelling of Antigenomic Therapy of Mitochondrial Diseases by Mitochondrially Addressed RNA Targeting a Pathogenic Point Mutation in mtDNA

### Example 7

### Design of Anti-Replicative RNA Molecules

As it was reported previously, the helix-loop domains of FD RNA can serve as signals for RNA mitochondrial import, and this molecule can be used as a vector to deliver various sequences into the organelles. Previously inventors also demonstrated that only one D-ARN structure can be sufficient for mitochondrial targeting of recombinant molecules to mitochondria in living cells.

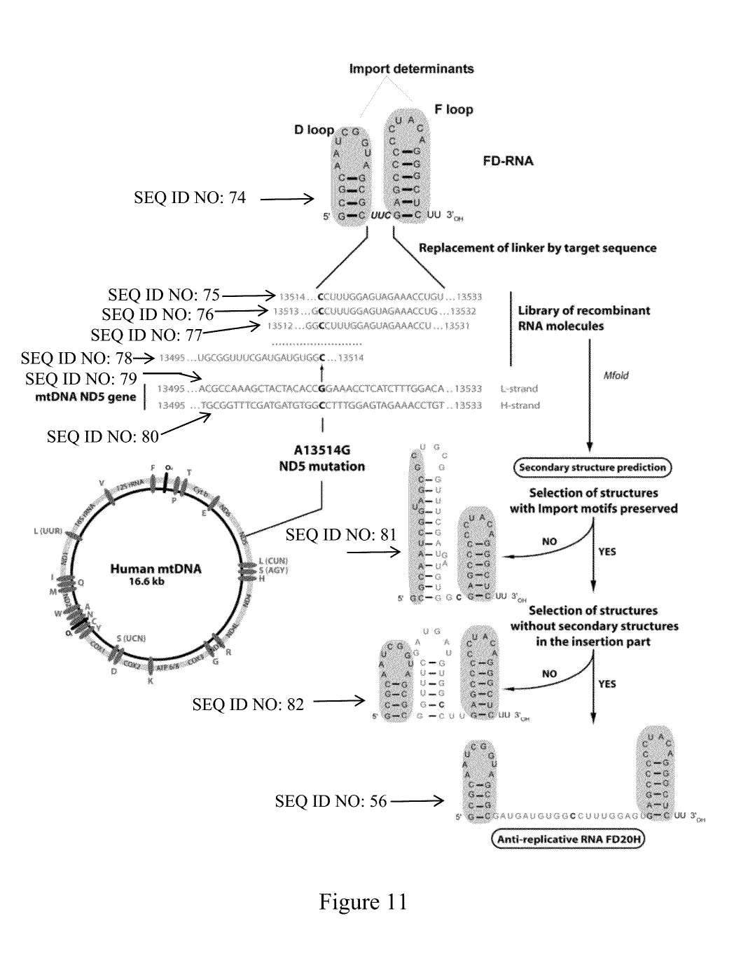

To apply the anti-genomic strategy to point mutations, the inventors use as a model the A13514>G mutation inducing amino acid replacement D393>G in the ND5 gene of human mtDNA, which encodes one of the membrane domain subunits of the respiratory complex I (29). This mutation was initially found in two unrelated patients with MELAS-like syndrome (18). To target the mutant mitochondrial genome, a series of RNA molecules were constructed, bearing sequences complementary to a mutated region of the ND5 gene inserted between two helix-loop domains of a short artificial FD RNA (FIG. 11).

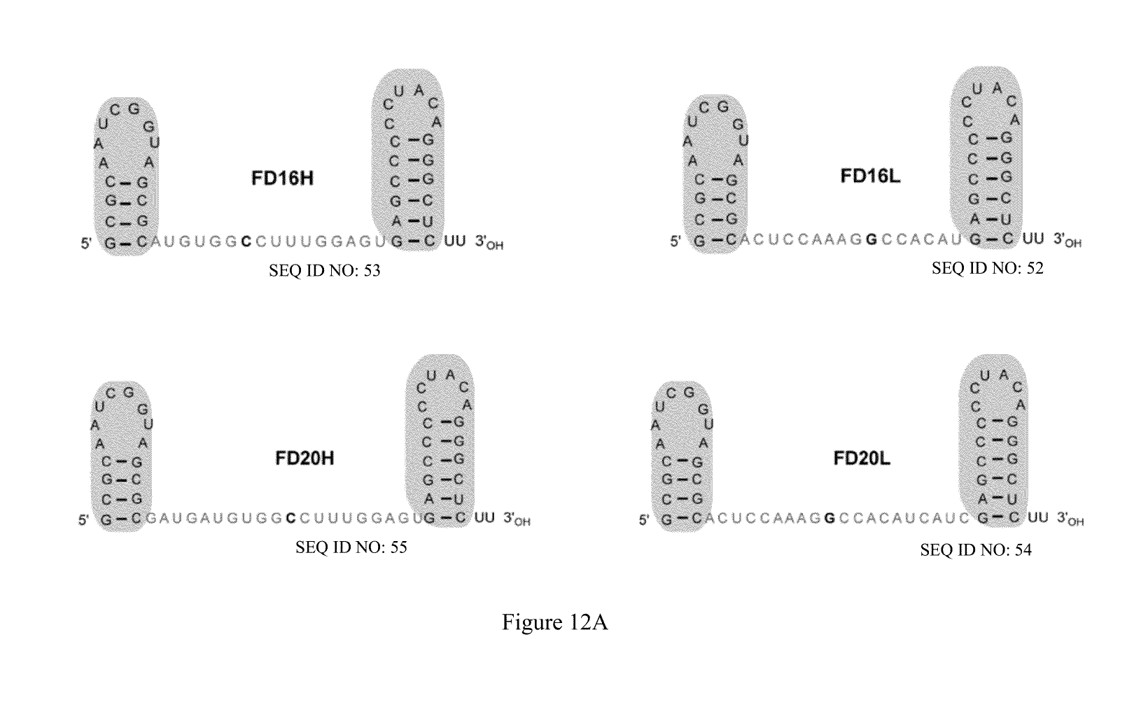

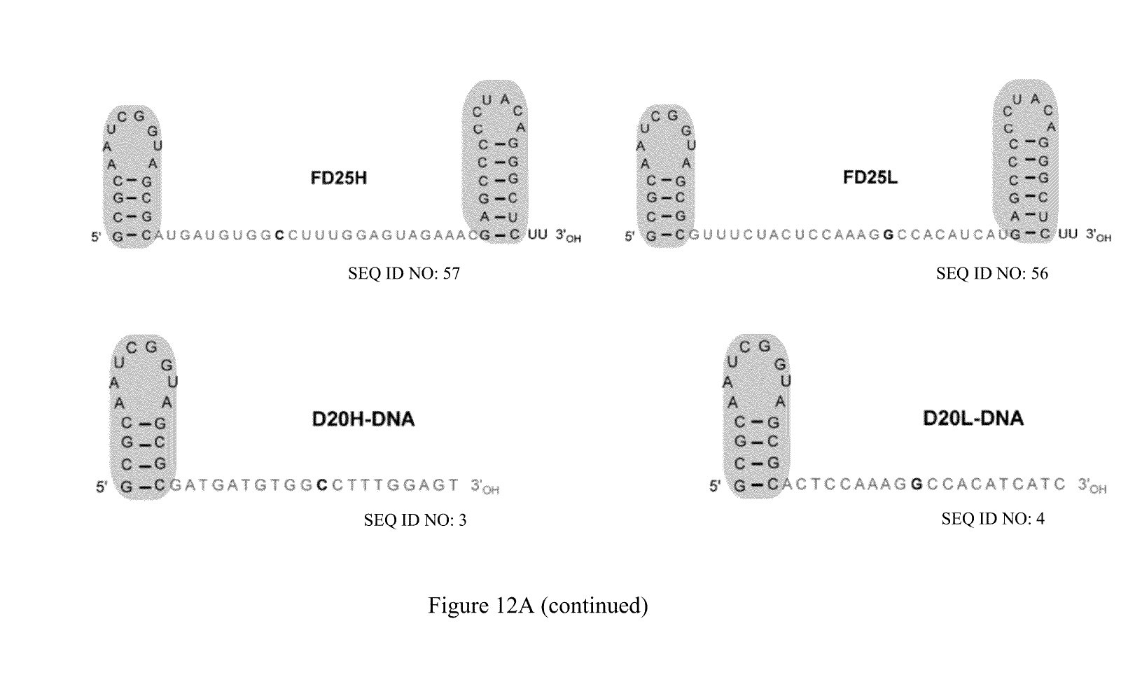

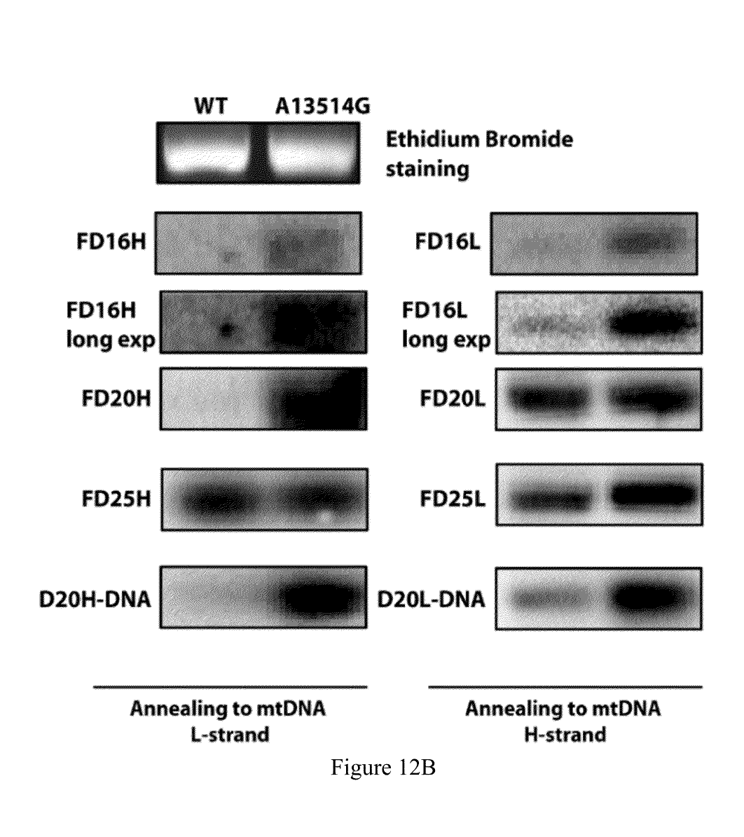

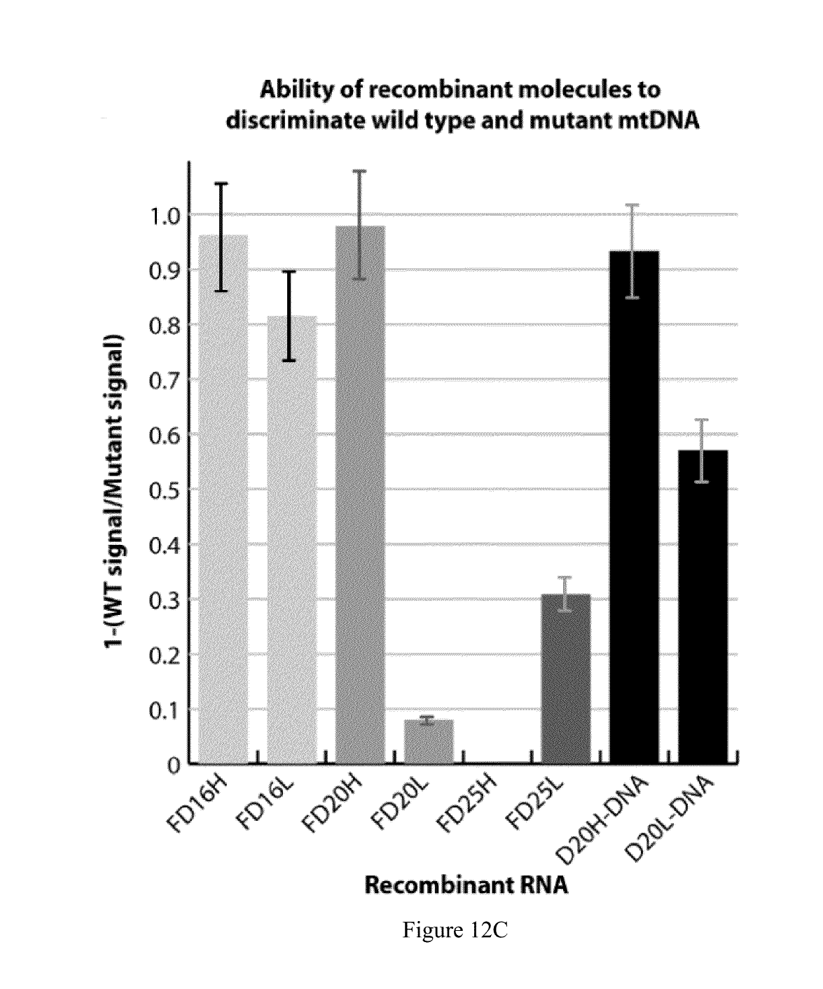

Firstly, the inventors analysed the secondary structure predictions for FD RNA molecules bearing insertions of 16, 20 and 25 nucleotides corresponding to a fragment of the ND5 gene, referred to as FD16L, FD16H, FD20L, FD20H, FD25L and FD25H; R for sequence of H-strand and S- for L-strand of mtDNA. For each sequence, a series of insertions bearing nucleotide G or C corresponding to the mutation A13514>G (for either H or L strand) in various positions (FIG. 11) were analyzed by several softwares (see Materials and Methods) with similar results. Only molecules with a low probability of alternative folding were retained for further studies (FIG. 10). To check the ability of the selected versions for a specific annealing with mutated, but not wild-type mtDNA, labelled recombinant RNAs were hybridized under physiological conditions with PCR-amplified mtDNA fragments either containing the mutation or not (FIG. 11). Versions FD25L and FD25H were not able to discriminate between mutant and wild-type mtDNA; the shorter RNA molecule FD20H (but not FD20L) demonstrated specific annealing with the A13514G bearing mtDNA but not with the wild-type one Annealing of molecules FD16H and FD16L was rather specific for mutant mtDNA; however, the efficiency of their hybridization at 37° C. was reproducibly lower when compared to FD20H. Inventors also tested chimeric molecules containing a stem-loop RNA import determinant and DNA-inserts corresponding to FD20H and FD20L sequences, referred to as D20H-DNA and D20L-DNA (FIG. 12). Chimeric RNA-DNA molecule D20L-DNA demonstrated a higher ability to discriminate between mutant and wild-type mtDNA when compared to FD20L.

Thus, the recombinant molecules with 20 nucleotide insertions (FD20L, FD20H, D20L-DNA and D20H-DNA) have been selected for the further analysis.

### Example 8

### Mitochondrial Import of Recombinant RNA

To study the localization of recombinant RNA molecules in cultured human cells, an approach was developed consisting of cell transfection with Alexa Fluor-488 labelled RNA FD20H and its subsequent co-localization with the mitochondrial network by means of fluorescent confocal microscopy (FIG. 13). RNA molecules (green fluorescence) were detectable in cells 24 h after transfection (Day 1) as green dots, most probably representing the RNA-Lipofectamine complexes, with only slight co-localization with the mitochondria (red fluorescence). In 2-4 days after transfection, the distribution of the green label drastically changed, the amount of dots was reduced, and RNA molecules were now mostly dispersed within the cell, displaying clear partial co-localization with the mitochondrial network (FIG. 3B and Supplementary video material).

To perform quantitative co-localization analysis of confocal microscopy images, the inventors estimated the values of Pearson's correlation coefficient and Manders' overlap coefficients M1 and M2 (25). For control RNA which is not imported into mitochondria (27), all the coefficients' values were very low (FIG. 12A), thus excluding the coincidental overlap of green and red signals. For FD20H RNA, the M1 values indicated that 2 days post transfection, approximately 50% of green fluorescence (RNA) have been co-localized with the red one (mitochondria), and this level did not significantly change between 2 and 4 days post transfection. M2 values, representing the percentage of the red fluorescence (mitochondria) overlapping with the green one (RNA), indicate that only 10% of mitochondria contained RNA 2 days post transfection. This degree has been increased in 3 and 4 days up to 70%, probably due to the mitochondrial dynamic events, fusion and fission, resulting in more homogeneous RNA distribution. These data clearly indicate mitochondrial targeting of FD20H RNA in living human cells.

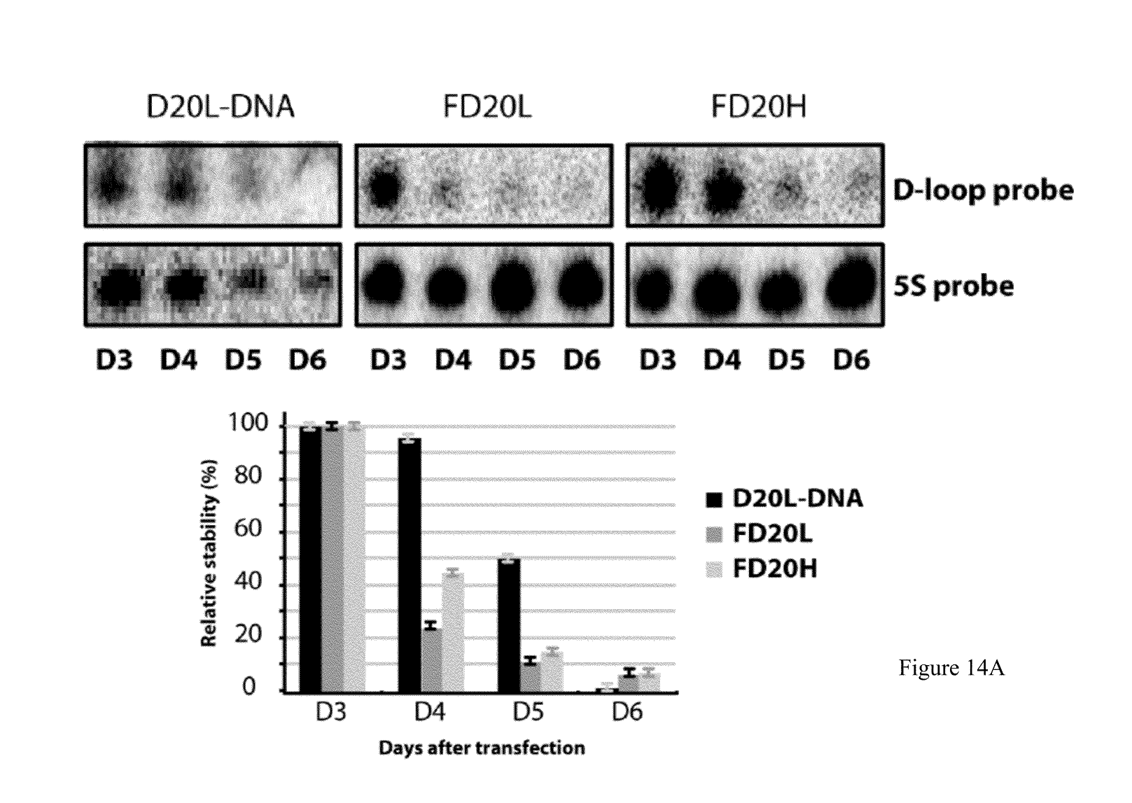

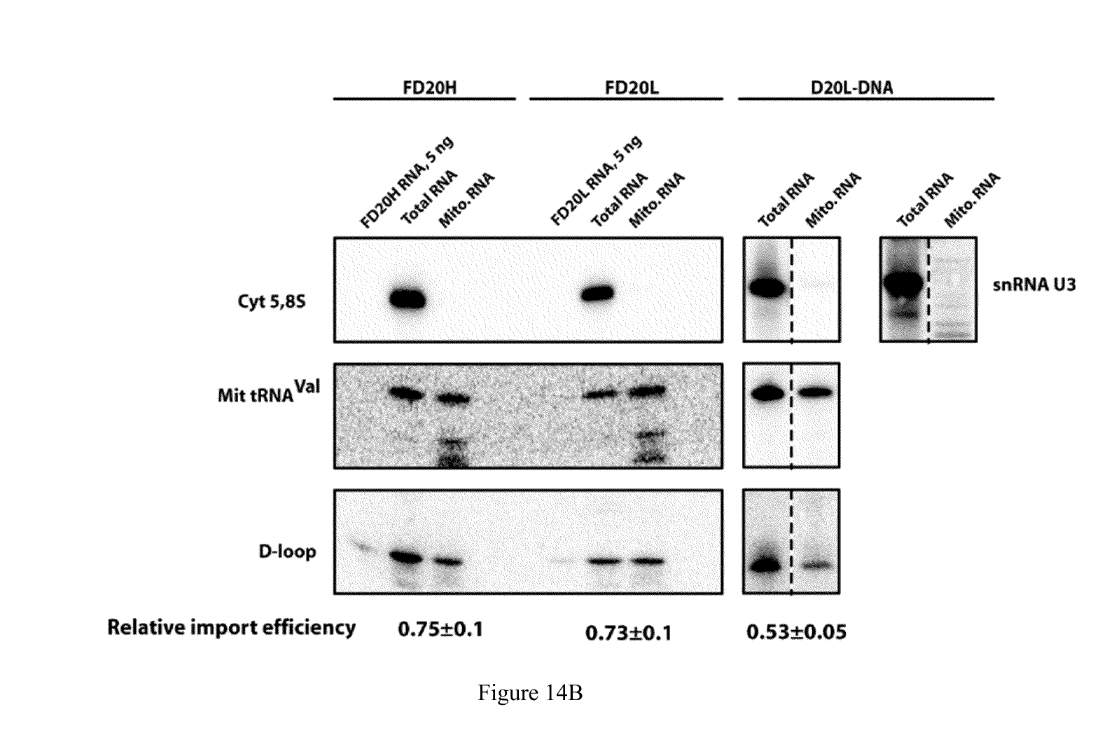

To measure the recombinant RNAs' stability in cultured cells and the efficiency of their import into the mitochondrial matrix, cybrid cells were transfected with in vitro synthesised recombinant RNAs and chimeric molecules. Their degradation rate in transfected cells was evaluated by Northern hybridization of total cellular RNA at different time points spanning a 6-day period after transfection (FIG. 14A). Recombinant molecules were detectable in cybrid cells at least 6 days after transfection. The chimeric version D20L-DNA revealed to be more stable than FD20L and FD20H. Such result suggests that the “insertion” part, lacking strong secondary structures, gives a prominent impact on the degradation of recombinant molecules. Thus, recombinant molecules in which RNA insertions were replaced by DNA sequences demonstrated improved stability in the living cells.

Since the microscopy data can give only a rough indication of the mitochondrial import of RNA molecules, Northern blot hybridization experiments were performed. For this, cells were transfected with purified recombinant molecules. The mitochondrial import was analyzed by hybridization of the whole cell RNA and RNA isolated from purified and RNase treated mitoplasts (mitochondria where the outer membrane was removed by digitonin) as described previously (14,21,28). The absence of signal in the mitochondrial RNA preparation after hybridization with the probe against the cytoplasmic 5.8S rRNA indicates that the treatment of mitochondria with ribonuclease and digitonin removed all contamination by cytoplasmic RNA (FIG. 14B). Hybridization signals obtained with the D-loop probe specific for all the recombinant molecules were normalized to the amounts of loaded RNA, estimated by hybridisation with a probe to a mitochondrial transcript (mit tRNAVa1). The data show that the import efficiencies of RNA molecules FD20H and FD20L were close to each other; import of the chimeric molecule D20L-DNA was only slightly decreased (FIG. 14B).

### Example 9

### Imported RNA can Shift Point Mutation Heteroplasmy Level in Cybrid Cells

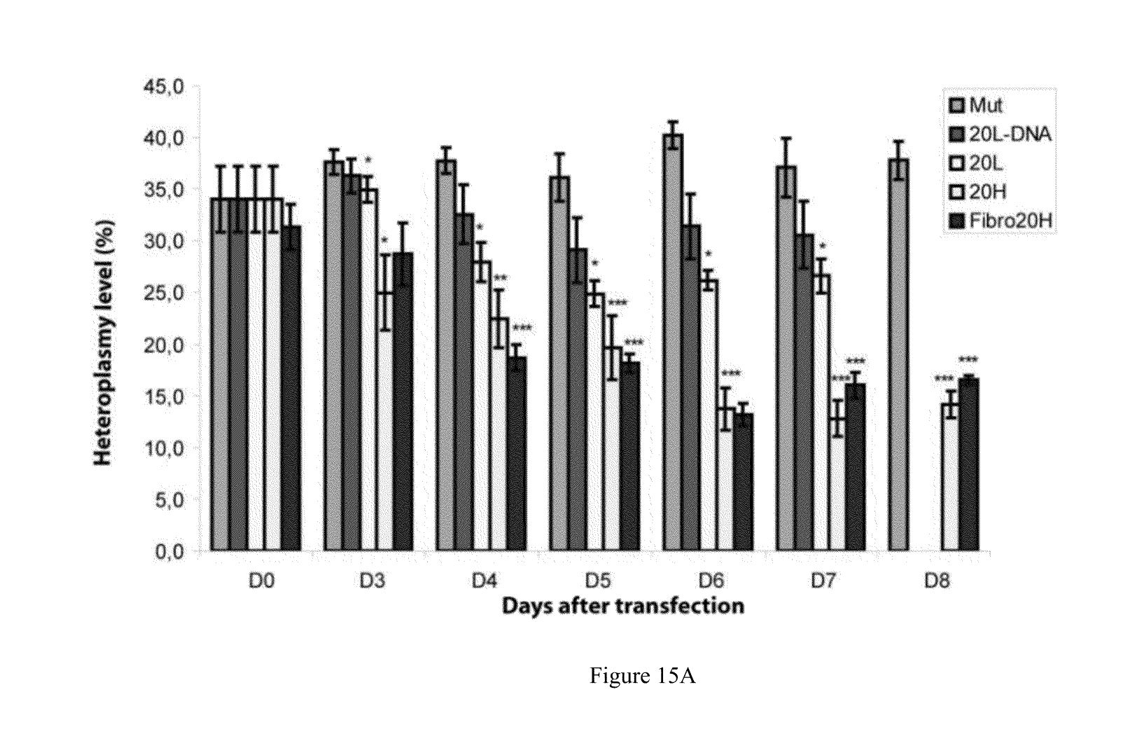

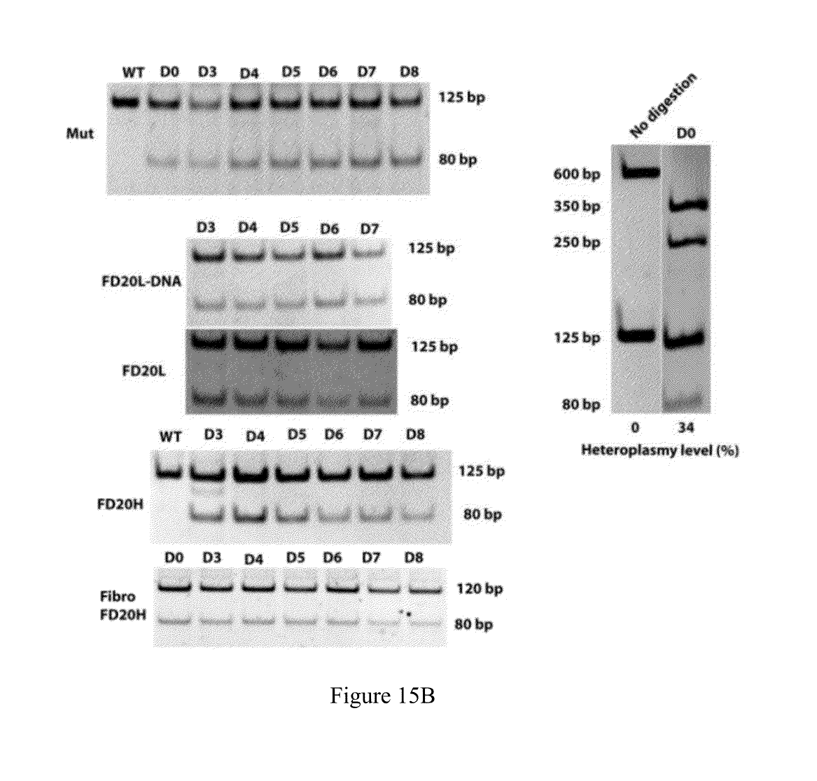

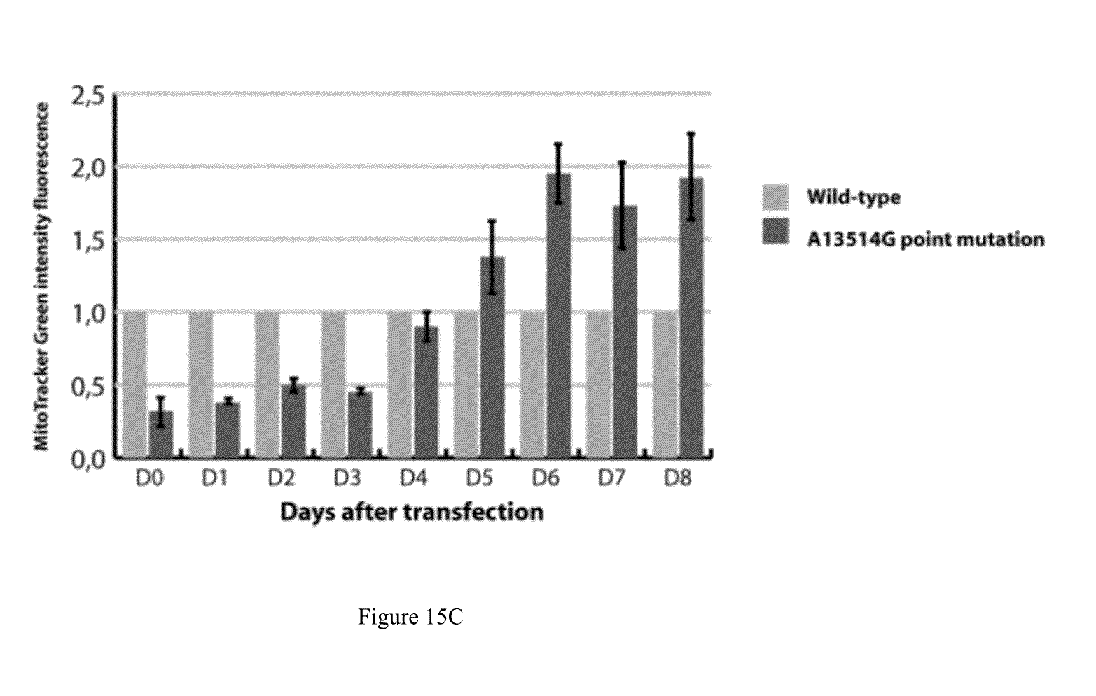

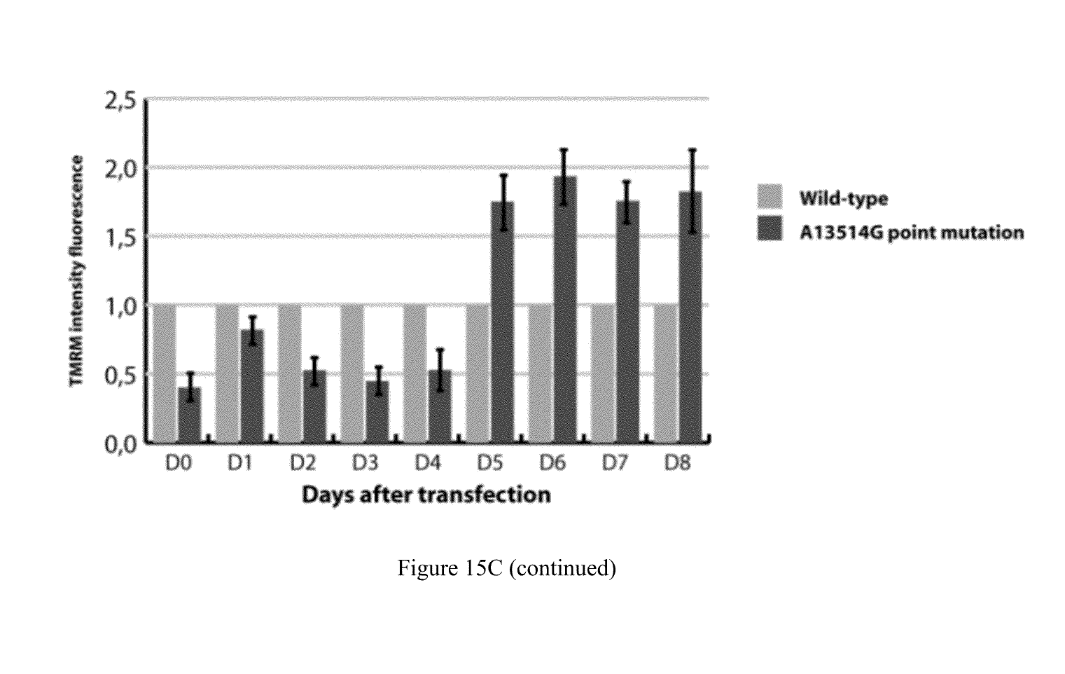

Transmitochondrial cybrid cell lines containing patient's mitochondria with 35% of mtDNA molecules bearing A13514G mutation (referred thereafter as ND5 mutation) were transfected with in vitro synthesised recombinant RNA and chimeric RNA-DNA molecules. Heteroplasmy levels (percentage of mutant ND5 mtDNA to all mtDNA molecules) were measured at different time points after transfection by RFLP analysis of PCR-amplified mtDNA fragment (FIG. 15A,B). The inventors observed a reproducible decrease of the proportion of mutant mtDNA in cells transfected by recombinant RNA containing FD20H inserts. The heteroplasmy shift became visible 3 days after transfection, then the heteroplasmy continued to decrease and reached the stable level of 13±2% in 6 days post transfection. To affirm these data, the inventors performed transfection of patient's fibroblasts, harbouring the m.13514A>G mutation, with FD20H RNA (FIGS. 15A,B). This completely independent experiment on primary human cells gave the same result of heteroplasmy shift, as obtained on the cybrid cells model.

Remarkably, in cells transfected with FD20H RNA, the decrease of mutant mtDNA correlated to an increased amount of mitochondria per cell measured as MitoTracker Green fluorescence (FIG. 15C). Moreover, the same increase of the fluorescence was detected by TMRM dye that is readily sequestered by active mitochondria, indicating a fully energized state of mitochondrial membranes (30).

In parallel experiments, mock-transfected cybrid cells demonstrated no heteroplasmy shift (FIGS. 15A,B). Cell transfection with FD20L RNA led to a very small heteroplasmy decrease, reaching the level of 27±5% in 7 days (FIG. 15A). Different effects of FD20H and FD20L RNA molecules are in perfect correlation with the more specific hybridization of FD20H RNA with mutant mtDNA (FIG. 12). Surprisingly, chimeric molecule D20L-DNA, characterized by a rather specific hybridization with mutant mtDNA in vitro, high stability in cells and efficient import into mitochondria, was not able to influence the heteroplasmy level.

Our data show that mitochondrially imported RNA (but not DNA) molecules can function as anti-genomic agents in human cells, affecting the amount of mitochondrial genomes bearing a point mutation.

Conclusion 3: Modelling of Antigenomic Therapy of Mitochondrial Diseases by Mitochondrially Addressed RNA Targeting a Pathogenic Point Mutation in mtDNA

Specific Annealing of RNA to mtDNA Bearing a Point Mutation

The anti-replicative approach aiming to shift heteroplasmy levels in mtDNA below the pathogenic threshold has been recently applied to cells containing a large 7 kb deletion in mtDNA (14). In this case, a new sequence generated at the fusion of the deletion boundaries has been inserted into RNA vector molecules, and specific annealing only with the mutant but not with wild-type mtDNA was demonstrated for recombinant RNA molecules. The inventors investigated whether this strategy can be extended to point mutations in the mitochondrial genome. The first question we addressed was: could a recombinant RNA importable into human mitochondria anneal in a specific way with only the mutant mtDNA containing a point mutation, but not with wild-type one?

Using Southern hybridization under conditions designed to approximate the intracellular ionic environment, the inventors demonstrated that the sequence of 20 nucleotides corresponding to H-strand of mutated (A13514G) region of mtDNA (FD20H) can discriminate between fragments of mutant and wild-type mtDNA. This was rather surprising, since the melting temperature predictions for hybrids between recombinant RNAs and mutated or wild-type mtDNA (Table 2) show Tm values above 37° C. Given that, all RNA molecules tested are likely to anneal to mutant and wild-type mtDNA fragments in the permissive conditions applied. According to the Tm predictions, we started our experiments with shorter insertions of 8-10 nucleotides, which should anneal to mutant but not to wild-type mtDNA. Surprisingly, we were not able to detect any signal with these RNA molecules used as probes for Southern blot hybridization, and no effect on heteroplasmy level in transfected cells (not shown).

Then, we gradually increased the length of the complementary part from 11 to 25 nt and obtained specific hybridization (FIG. 12) for molecules containing 16 and 20 nt insertions. We suppose that the discrepancy between predictions and experimental data is due to the absence of software adapted to calculate the Tm of RNA-DNA duplexes containing a mismatch. Another possible explanation consists of formation of alternative secondary structures in RNA molecules which cannot be predicted by available software.

Another interesting issue: hybridization of RNA containing an insertion of the same length corresponding to the complementary L-strand of mtDNA (FD20L) was much less selective. This discrepancy can be explained by different base pairing: for FD20H, mismatch C*A decreases the Tm of hybridization with wild-type mtDNA by 10° C. (Table 2) compared to mutant mtDNA (predicted by IDT Sci-Tools OligoAnalyser 3.1 software for mismatch-containing hybrids). In contrast, for FD20L, a corresponding mismatch is G-T, which decreases Tm by only 5.5° C., allows annealing of FD20L RNA with both mtDNA fragments at 37° C. The above data show that a point mutation can be selectively addressed by RNA containing a 20 nucleotide stretch; however, for transitions A->G (in L-strand of mtDNA), sequences corresponding to H-strand would have a more selective effect and vice versa, for G->A transitions, only L-strand sequences should be used.

Pathogenic Mutation in ND5 Gene

mtDNA mutation A13514>G, which we use as a model in the present study, is localized in NADH dehydrogenase ND5 subunit of the respiratory complex I. This is the first and largest enzyme of the respiratory chain (45 subunits, total size are about 1 MDa), coupling electron transfer between NADH and ubiquinone to the translocation of four protons across the inner mitochondrial membrane (31). The L-shaped complex consists of hydrophilic and membrane domains. The three largest transmembrane subunits ND2, 4 and 5 at the far end of the membrane arm are homologous to each other and likely participate in the conformation-coupled proton translocation (32). Mutation A13514>G induces amino acid replacement D393>G localized in a very conserved region of ND5 transmembrane helix 12 (33,34), forming the second half of the proton channel, and involved in a cascade of conformational changes leading to proton translocation. Therefore, any mutation exchanging the negatively charged D393 to uncharged amino acid residue could disturb the transport of the proton through the ND5 subunit channel of respiratory complex I.

The ND5 gene turned out to be frequently mutated (29). Point mutation A13514>G inducing amino acid replacement D393>G has been detected in four unrelated patients with MELAS-like and Leigh syndromes (18,35,36). Another transition, the frequently reported and well-documented G13513>A, affects the same D393 residue in the ND5 gene, but the amino acid replacement is different, D393>N (reviewed in (29)). A prominent clinical feature detected in patients with these mutations was a visual loss due to optic atrophy, indicating an exquisite sensitivity of the optic nerve to damage caused by alteration of the ND5 D393 residue (37). Thus, search for D393 mutations has been proposed to be a part of the routine screening for mitochondrial disorders (18).

More recently, among the patients with Leigh-like syndrome and D393>N mutation in ND5, several cases were reported characterized by normal complex I activity in muscle (35,38). The authors suggested that the relatively low mutant heteroplasmy and normal respiratory chain activities in muscle do not necessarily represent the situation in affected tissue such as brain and/or optic nerve. Thus, even low mutation load in D393 in the presence of normal respiratory chain analysis may be considered as pathogenic (35), illustrating the complexities of correlating A13514>G heteroplasmy levels with biochemical phenotype in a patient's muscle and fibroblasts. This can be a characteristic feature of NADH dehydrogenase complex, since it was recently demonstrated that even a low dose of wild-type ND6 gene is sufficient to drive assembly of near normal levels of complex I (1). All these data taken together indicate that a low mutant load of mutation m.13514A>G might cause a functional defect of ND5 protein, but this defect would be very low and hardly noticeable. This is exactly the case of cells used in the present study.

Effect of Imported RNAs on mtDNA Heteroplasmy

As we have shown previously, mitochondrially imported anti-replicative RNAs can cause a replication stalling at the site of RNA annealing to the mutant mtDNA due to impairing of the replication fork progression (14). Nevertheless, we obtained only a transient shift of heteroplasmy in cells bearing a large deletion in mtDNA, the initial heteroplasmy level being restored in 6-8 days after cell transfection with anti-replicative RNA. This can be explained by recombinant RNA degradation, followed by preferential replication of shorter mitochondrial genomes bearing deletion, due to the previously reported effect of “replicative advantage of deleted mtDNA” (39). In the present study, we successfully obtained a stable decrease of the proportion of mtDNA molecules containing ND5 point mutations after transfection of cybrid cells and primary fibroblasts with anti-replicative RNA FD20H (FIG. 15). Since no difference was expected in the rate of replication for wild-type and mutant mtDNA, we can suggest that even after degradation of recombinant RNA, the induced shift in ND5 point mutation heteroplasmy level may become stable. The maintenance of wild-type mtDNA enrichment was previously reported for several mutations (40,41).

Among several anti-replicative recombinant molecules characterised by comparable stability in cells and mitochondrial import efficiency (FIG. 14), only one, FD20H, was able to induce a prominent heteroplasmy shift. This RNA molecule demonstrated the highest ability to discriminate between mutant and wild-type mtDNA (FIG. 11). Chimeric molecule, containing the same sequence of the insertion part as FD20H, but in the form of DNA, was not capable to shift the heteroplasmy level (FIG. 14). These data provide an additional proof of recombinant RNA action at the level of mtDNA replication, since it was suggested that mitochondrial replisome helicase can separate DNA-DNA hybrids, but not regions of short RNA-DNA hybrids in the mtDNA D-loop (42). Thus, only mitochondrially imported RNA (but not DNA) molecules are likely to function as anti-replicative agents in human mitochondria.

Cybrid cells used in the present study, containing 35% of mtDNA molecules bearing the A13514>G mutation, were almost asymptomatic, characterized by a very slight decrease of all the measurable parameters as oxygen consumption, levels of ATP and ROS and complex I enzymatic activity compared to wild-type 143B cell line (not shown). So far, it observed a prominent decrease of amount of mitochondria in cybrid cells, measured by use of MitoTracker Green, a mitochondria-selective fluorescent label, accumulating in the matrix (FIG. 5C) (43). Notably, transfection with anti-replicative RNA FD20H, leading to an important decrease of the level of ND5 point mutations, caused, at the same time, a recovery of mitochondrial content. Unexpectedly, if the MitoTracker Green fluorescence increased gradually from day 4 to day 6 post transfection and then reached a plateau, the shift of the TMRM fluorescence between days 4 and 5 was rather sharp. Thus, the inventors observed some temporary hyperpolarization of mitochondria only at day 5 post transfection. We can hypothesise that following the heteroplasmy decrease observed for ND5 cybrid cells in 3-4 days post transfection, the synthesis of increased amounts of normal, fully active ND5 protein might create a temporary unbalance in the assembly of respiratory complexes resulting in a rise of the basal production of ROS (at the 5th day) and increasing mitochondrial biogenesis (days 6-8).

All data indicate the possibility to obtain a curative effect of mitochondrial dysfunctions in human cells by a heteroplasmy shift induced by short RNA molecules targeted into mitochondria. Although the inhibitory effect was partial, it may have a long term therapeutic interest, since only high levels of mutations in human mtDNA become pathogenic. The validation of the anti-replicative RNA strategy for a point mutation in mtDNA in cultured cybrid cells can be considered as an important step to further develop an efficient therapy of mitochondrial diseases.

TABLE 1
Predicted and measured melting temperatures for hybrids between synthetic
oligonucleotides (SO) and mutant (KSS) or wild-type (WT mtDNA regions (SD = 2.7°).

| | | | | Homology | | Homology | |
|---|---|---|---|---|---|---|---|
| | | | | with WT | | with WT | |
| | | Measured | Predicted | mtDNA | | mtDNA | |
| | Homology | Tₘ for | Tₘ for | (5′ | Tₘ for 5′ | (3′ | Tₘ for 3′ |
| | with KSS | KSS | KSS | deletion | boundary | deletion | boundary |
| | mtDNA | mtDNA | mtDNA | boundary) | deletion | boundary) | deletion |
| SO | (b) | (° C.) | (° C.) | (b) | (° C.) | (b) | (° C.) |
| D22L | 22 | 50 | 52.1 | 12 | 34.4 | 11 | 33.5 |
| D22L- | 22 | 56 | 52.1 | 12 | 34.4 | 11 | 33.5 |
| Me | | | | | | | |
| D22L- | 22 | ND | 52.1 | 12 | 34.4 | 11 | 33.5 |
| 4Me | | | | | | | |
| D22L- | 22 | 56.5 | ND | 12 | ND | 11 | ND |
| Me-T | | | | | | | |
| D22L-F | 22 | 62 | ND | 12 | ND | 11 | ND |
| D22L- | 22 | 62.5 | ND | 12 | ND | 11 | ND |
| F-T | | | | | | | |
| D22L- | 22 | 61.5 | 62.8 | 12 | 45.3 | 11 | 43.5 |
| DNA | | | | | | | |
| D22L- | 22 | 61.5 | ND | 12 | ND | 11 | ND |
| DNA-T | | | | | | | |
| D13H- | 13 | ND | 50.8 | 7 | 21.2 | 7 | 20.2 |
| DNA | | | | | | | |
| D13L- | 13 | ND | 49.6 | 8 | 26.9 | 6 | 12.4 |
| DNA | | | | | | | |
| F13H- | 13 | ND | 49.6 | 6 | 12.4 | 8 | 26.9 |
| DNA | | | | | | | |
| F13L- | 13 | ND | 49.6 | 8 | 26.9 | 6 | 12.4 |
| DNA | | | | | | | |

TABLE 2
Melting temperature predictions for hybrids between recombinant
RNAs and mutated or wild-type (WT) mtDNA regions.

| | Insert | | | |
|---|---|---|---|---|
| RNA | (bp) | Tm for mutated DNA | Tm for WT DNA* | Δ Tm |
| FD16L | 16 | 48.4° C. | 40.2° C. | 8.2° C. |
| FD16H | 16 | 48.4° C. | 35.0° C. | 13.4° C. |
| FD20L | 20 | 54.9° C. | 49.3° C. | 5.6° C. |
| FD20H | 20 | 52.8° C. | 42.9° C. | 9.9° C. |
| FD25L | 25 | 59.0° C. | 54.9° C. | 4.1° C. |
| FD25H | 25 | 59.0° C. | 52.2° C. | 6.8° C. |

*predicted by IDT Sci-Tools OligoAnalyser 3.1 software for mismatch containing DNA-DNA hybrids. Δ Tm, predicted decrease of Tm for wild-type mtDNA comparing to mutated mtDNA.

### Materials and Methods

Plasmids and Antibodies

Plasmid pDEST17 expressing human mitochondrial lysyl-tRNA synthetase (KARS2) was kindly provided by M. Sissler (IBMC, Strasbourg). To produce the precursor of mitochondrial KARS2 (preKARS2) protein, the Quick change mutagenesis kit (Stratagene) was used to insert the mitochondrial targeting sequence at the N-terminus of the mitochondrial KARS2 protein. A thrombin cleavage site and a 6×histidine tag at the N-terminus were deleted and a 6×histidine tag was inserted at the C-terminus using the same approach. For this, the following oligonucleotides were used:

Mitochondrial Targeting Sequence Insertion
(SEQ ID NO: 18)
5′GCCACGCGGTTCTTTGACGCAAGCTGCTGTAAGGCTTGTTAGGGGGTC
CCTGCGCAAAACCTCCTGGGCAG3′
(SEQ ID NO: 19)
5′CTGCCCAGGAGGTTTTGCGCAGGGACCCCCTAACAAGCCTTACAGCAG
CTTGCGTCAAAGAACCGCGTGGC3′
Thrombin Cleavage Site and 6XHis Tag Deletion
(SEQ ID NO: 20)
5′CTTTAAGAAGGAGATATACATATGTTGACGCAAGCTGCTGTAAGG3′
(SEQ ID NO: 21)
5′CCTTACAGCAGCTTGCGTCAACATATGTATATCTCCTTCTTAAAG3′
His Tag Insertion at the C-terminus of preKARS2
(SEQ ID NO: 22)
5′CAACAGTTGGCAGTTCTGTCCACCATCACCATCACCATTGAGACCCA
GCTTTCTTGTAC3′
(SEQ ID NO: 23)
5′GTACAAGAAAGCTGGGTCTCAATGGTGATGGTGATGGTGGACAGAAC
TGCCAACTGTTG3′

pTRE2hyg plasmid expressing preKARS2 and antibodies directed against the residues 25 to 42 of the preKARS2 protein described in [25] were kindly provided by Marc Mirande (Gif-sur-Yvette, France). Polyclonal antibodies against human actin were from Santa Cruz Biotechnology.

Purification of the Recombinant preKARS2 Protein

To obtain the recombinant KARS2 and preKARS2 proteins, Escherichia coli strain BL21 codon plus (DE3)-RIL cells (Stratagene) were transformed with the pDEST17 plasmid. The transformed cells were grown in 500 ml of LB medium to a cell density corresponding to OD600=0.6, then the protein expression was induced for 2 h at 37° C. by addition of 0.5 mM isopropyl b-D-1-thiogalactopyranoside (IPTG) to the bacterial culture. The cells were harvested by centrifugation at 6000 g for 10 min, lysed with 1 mg/ml of lysozyme on ice for 30 min and then sonicated thrice for 20 sec in the buffer consisting of 50 mM NaH2PO4, 300 mM NaCl and 20 mM imidazole. The cell lysate was centrifuged at 10,000 g for 15 min and the pellet was solubilized in the denaturing buffer consisting of 100 mM Tris-HCl (pH 8), 100 mM NaH2PO4, 10 mM imidazole and 8 M urea. This was followed by centrifugation at 12000 g for 15 min and the supernatant was applied to a Ni-NTA column (Qiagen) for 2 h at 4° C. After binding, the column was washed three times with the denaturing buffer containing 20 mM imidazole to eliminate weakly bound bacterial proteins. The recombinant preKARS2 protein was eluted from the column with 200 mM imidazole, refolded by stepwise elimination of urea and finally dialyzed against 50 mM Tris-HCl (pH 8), 300 mM NaCl and 40% glycerol and stored at −20° C. The purity of the protein was checked by sodium dodecyl sulfate-polyacrylamide gel electrophoresis (SDSPAGE) with Coomassie blue staining The recombinant yeast enolase Eno2p was isolated as described previously [10]. Enolase from rabbit muscles was from Sigma-Aldrich.

Recombinant RNA Modelling

To predict secondary structures of recombinant RNA molecules and estimate their free energies (dG), the Mfold program [46,47] and IDT Sci-Tools OligoAnalyser 3.1 software [48] were used.

RNA Synthesis and Purification

The yeast tRK1 T7-transcript was obtained as described [32]. For small artificial RNAs, PCR amplification of the following oligonucleotides containing a T7 promoter at the 59-end (underlined) was performed:

T7 + FD-L
(SEQ ID NO: 24)
TAATACGACTCACTATAGCGCAATCGGTAGCGCCTCTTTACAGTGCTTAG
TTCTCGAGCCCCCTACAGGGCTCTT
FD-L
(SEQ ID NO: 28)
GCGCAAUCGGUAGCGCCUCUUUACAGUGCUUAGUUCUCGAGCCCCCUACA
GGGCUCUU
T7 + FD-H
(SEQ ID NO: 25)
TAATACGACTCACTATAGCGCAATCGGTAGCGCAGTAAGCACTGTAAATG
AGCCCCCTACAGGGCTCTT
FD-H
(SEQ ID NO: 29)
GCGCAAUCGGUAGCGCAGUAAGCACUGUAAAUGAGCCCCCUACAGGGCUC
UU
T7 + HD:
(SEQ ID NO: 26)
TAATACGACTCACTATAGCGCAATCGGTAGCGCCTCTTTACAGTGCTTAG
TTCTC
HD
(SEQ ID NO: 30)
GCGCAAUCGGUAGCGCCUCUUUACAGUGCUUAGUUCUC
T7 + HF:
(SEQ ID NO: 27)
TAATACGACTCACTATAGGTCTTTACAGTGCTTACTTCTCGAGCCCCCTA
CAGGGCTCCA
HF
(SEQ ID NO: 31)
GGUCUUUACAGUGCUUACUUCUCGAGCCCCCUACAGGGCUCCA

RNA transcripts were obtained in vitro using the Ribomax kit (Promega). Following transcription, the DNA template was removed by digestion with RQ1 RNase-Free DNase (Promega). RNAs were purified by 12% PAGE with 8 M urea and eluted from the gel with the RNA extraction buffer containing 0.5 M CH3COONH4, 10 mM Mg(CH3COO)₂, 0.1 mM EDTA and 0.1% SDS. The eluted RNA was precipitated with ethanol.

Electrophoretic Mobility Shift Assay (EMSA)

Purified RNA was dephosphorylated with alkaline phosphatase (Boehringer Mannheim) and labeled at the 59-end with c-32P-ATP using T4 polynucleotide kinase (Promega). The labeled RNA was denatured at 100° C. and then slowly cooled down to the room temperature. For RNA binding assays, the appropriate amount of protein and labeled RNA were mixed in 20 ml of the buffer containing 20 mM Tris-HCl pH 7.5, 150 mM NaCl, 10 mM MgCl2, 5 mM DTT, 10% glycerol, 0.1 mg/ml BSA and incubated at 30° C. for 15 min. The mixture was separated by native 8% PAGE in 0.56Tris-borate buffer (pH 8.3) and 5% glycerol, followed by Typhoon-Trio (GE Healthcare) scanning and quantification as described in.

North-Western Blot Hybridisation

Recombinant pre-KARS2 was loaded on 10% SDS-PAAG and blotted to nitocellulose membrane. The membrane was incubated in 0.1 M Tris-HCl, 20 mM KCl, 2.5 mM MgCl2, 0.1% Nonidet P40, pH7.5, at 4° C. for 1 h with stirring, then washed several times with the same solution and blocked in 10 mM Tris-HCl, pH7.5, 5 mM Mg(CH3COO)₂, 2 mM dithiothreitol, 2% BSA, 0.01% Triton X-100 for 5 min at 25° C. Then, the membrane was incubated for 2 h at 4° C. in the import buffer without sorbitol, containing 1 nM [32P]-labelled RNA, as in, washed with the same buffer without RNA and analysed by Typhoon-Trio (GE Healthcare) scanning and quantification.

In Vitro Import Assay

Mitochondria were isolated and verified for intactness as described. The standard in vitro import assay into isolated mitochondria was performed as in Entelis et al., J. Biol. Chem., 276, 45642-45653. For this, purified HepG2 mitochondria were incubated with radioactively labeled RNA and purified proteins in the import buffer: 0.6 M sorbitol, 20 mM HEPES-KOH (pH 7), 10 mM KCl, 2.5 mM MgCl2, 5 mM DDT and 2 mM ATP. For a standard in vitro assay, we added 3 pmoles of labelled RNA per 0.1 ml of the reaction mixture containing 0.1 mg of mitochondria (measured by the amount of mitochondrial protein). This corresponds to the 100% RNA input. After incubation for 15 min at 34° C., 50 mg/ml of RNase A (Sigma) was added and the reaction was incubated for additional 15 min to digest all unimported RNA. The mitochondria were washed three times with the buffer containing 0.6 M sorbitol, 10 mM HEPES-KOH (pH 6.7) and 4 mM EDTA, then resuspended in 100 ml of the same buffer and treated with an equal volume of 0.2% digitonin (Sigma) solution to disrupt the mitochondrial outer membrane, followed by purification of mitoplasts. The mitoplast pellet was resuspended in the solution containing 100 mM CH3COONa, 10 mM MgCl2, 1% SDS and 0.05% diethylpyrocarbonate (DEPC), boiled for 1 min and RNA was extracted at 50° C. with water-saturated phenol. RNA was precipitated with ethanol and separated by 12% PAGE containing 8 M urea, followed by quantification with the Typhoon-Trio scanner using the Image Quant-Tools software (GE Healthcare). The amount of the imported RNA was determined by comparison of the band density of the protected full-sized RNA isolated from the mitoplasts after the import assay with an aliquot (2-5%) of the RNA input.

Human Cell Culture, Overexpression and Downregulation of preKARS2

HeLa Tet-Off cells stably expressing the tetracycline-controlled transactivator (tTA) were purchased from Clontech Laboratories Inc. The HepG2 and HeLa Tet-Off cells were maintained in the Dulbecco modified Eagle's medium (DMEM, Invitrogen) with high glucose (4.5 g/l) supplemented with 10% fetal calf serum, 100 mg/ml of streptomycin and 100 mg/ml of penicillin (Gibco). For induction of protein expression in HeLa Tet-Off cells, the Tet system approved fetal bovine serum from Clontech was used. The cells were cultivated in a humidified atmosphere at 37° C. and 5% of CO2.

For overexpression of preKARS2, HeLa Tet-Off cells were grown to the 60% confluency and transfected with the pTRE2hyg plasmid expressing preKARS2 using Lipofectamin 2000 (Sigma) according to the manufacturer's protocol. At the same time, the cells were transiently transfected with mitochondrially importable RNAs. After 48 h, the cells were analysed for the preKARS2 overexpression by Western blotting and for the RNA import by Northern hybridization. To downregulate preKARS2, two 21-mer siRNAs corresponding to the mitochondrial targeting sequence of the human preKARS2 mRNA were synthesized. The sequences of the sense strands of these siRNAs are as follows: siRNA1: 5′CAACTTGCTCCTTTCACAGCG 3′ (SEQ ID NO: 32) and siRNA2:5′ AAGGACAAGTCATTTTCTGAT3′ (SEQ ID NO: 33). As a negative control, a nonsilencing siRNA (Ref: SR-CL000-005, Eurogentec) was used. Our optimized protocol consisted of two subsequent transfections: firstly, HepG2 cells were transfected in suspension with 40 nM of each siRNA using the RNAiMax transfection reagent (Invitrogen), according to the manufacturer's protocol. 24 h later, the cells formed a monolayer and were transfected again with 40 nM of each siRNA using Lipofectamine 2000 (Invitrogen). The cells were grown for 40 h after the second siRNA transfection and then transfected with one of the mitochondrially importable RNAs. 3 days after the second siRNA transfection, the downregulation was analysed by Western blotting, and the RNA import by Northern hybridization.

RNA Import Assay In Vivo

For transfection of HepG2 and HeLa Tet-Off cells, 3 mg of RNA per 75 cm2 flask were used. Transfection was performed with the Lipofectamine 2000 reagent (Invitrogen), according to the manufacturer's protocol. After 48 h, the cells were detached, mitochondria were isolated and purified as described above. The total and mitochondrial RNA were isolated with the TRIzol reagent (Invitrogen), separated by 12% PAGE containing 8 M urea and analysed by Northern blot hybridization with 5′-3′ P labelled oligonucleotide probes: anti-tRK1 (1-34): GAGTCATACGCGCTACCGATTGCGCCAACAAGGC (SEQ ID NO: 35) to detect tRK1, FD-L, FD-H and HD RNA; anti-HF RNA probe: TGGAGCCCTGTAGGG (SEQ ID NO: 36); anti-mt tRNAVa1 probe: GTTGAAATCTCCTAAGTG (SEQ ID NO: 37) and anti-cyt 5.8S rRNA probe: AAGTGACGCTCAGACAGGCA (SEQ ID NO: 38). After quantification with the Typhoon-Trio scanner, the relative efficiency of the RNA import into mitochondria was calculated as a ratio between the signal obtained with the anti-tRK1 probe and that obtained with the probe against the host mitochondrial tRNAVa1, as described previously. Because it is rather difficult to normalize exactly the amount of mitoplasts isolated from various cell lines, we loaded on the gel the mitochondrial RNA isolated from the same number of cells, and then used the hybridization signals corresponding to the mitochondrial tRNAVa1 as a loading control. Thus, we take into account not the absolute intensity of hybridization signals but the ratios between the signals corresponding to the imported into mitochondria tRK1 (or FD-RNAs) and the host mitochondrial valine tRNA's gene transcript. To calculate the absolute import efficiencies for various RNAs, the total level of the RNAs in the transfected cells was taken into account. For this the relative import efficiencies were divided by the ratios calculated in the same way but for the total RNA preparations.

Immunoblotting

For Western immunodecoration, cells were lysed in the Laemmli buffer (50 mM Tris-HCl, pH 6.8, 2% SDS, 0.1% bmercaptoethanol, 0.01% bromophenol blue and 10% glycerol) for 10 min at 90° C., and 30 mg of protein was separated by 10% SDSPAGE. The proteins were electroblotted onto a nitrocellulose membrane and probed with a primary polyclonal antibody against preKARS2 and a commercially available polyclonal antibody against actin (G2308, Santa Cruz Biotechnology). Bands were visualized with anti-rabbit or anti-goat secondary antibodies conjugated with horseradish peroxidase using the ECL Plus Western Blotting detection reagent (GE Healthcare).

Transmitochondrial Cybrid Cell Line, Transient Transfection with RNA

Cultured skin fibroblasts derived from a patient diagnosed with a Kearns Sayre Shy syndrome were fused to a human rho0 osteosarcoma cell line (143B) as described. Cybrid cells used in this study contained 65±2% mutant mtDNA bearing a large deletion spanned from nucleotide 8363 to 15,438 thus removing 7075 base pairs including 9 structural genes and 6 tRNA genes. Transient transfection with synthetic RNAs was performed as described in state of arts with minor modification: for 2 cm² wells of 80%-confluent cells we used 0.25 mg of RNA. Transfections were performed with Lipofectamin-2000 in OptiMEM medium (Invitrogen), as described in the supplier's protocol. OptiMEM was changed to a standard DMEM medium the following day after the transfection. Transfection procedure did not lead to any detectable decrease in viability of the cells or significant change of the overall mtDNA amount (measured by qPCR) suggesting the absence of synthetic RNAs toxicity.

Mitochondria Isolation, Assays for RNA Stability and Mitochondrial Import

Mitochondria from cultured cybrid human cells were isolated as described previously by several rounds of high (20,000 g) and low (4000 g) centrifugations. After the second round of high-speed centrifugation of mitochondria, they were re-suspended in the breakage buffer (0.44M mannitol, 20 mM Tris HCl (pH 7.0), 20 mM NaCl, 1 mM EDTA) and centrifuged through two layers of sucrose (0.5 M and 1.5 M). Mitochondria were collected on the top of the 1.5 M layer and harvested by high-speed centrifugation. Mitochondria were treated with RNase A and digitonin to get rid of nonspecifically attached RNA. Total and mitochondrial RNA were isolated with TRIzol reagent (Invitrogen). Stability and mitochondrial import of synthetic RNA molecules were analysed by Northern hybridization of total and mitochondrial RNA with 32P-labelled oligonucleotide probes against synthetic RNAs or against control cytosolic and mitochondrial RNA. The following probes were used: D-loop (5′-GAGTCATACGCGCTACCGATTGCGCCAACAAGGC-3′) (SEQ ID NO: 39); 5,8S (5′-GGCCGCAAGTGCGTTCGAAG-3′) (SEQ ID NO: 40); 5S (5′-CATCCAAGTACTACCAGGCCC-3′) (SEQ ID NO: 41); tRNAThr (5′-TCTCCGGTTTACAAGAC-3′) (SEQ ID NO: 42); and KSS part (5′-GCTAAGTAAGCACTGTA-3′) (SEQ ID NO: 43). To compare the stability of different recombinant RNA, the relative concentration (R0, Rt) of each RNA in various time periods (t) after transfection was calculated as a ratio between the specific probe signal and the signal for 5S rRNA probe. The half-life period of RNA was calculated as t1/2=ln 2/k, where the degradation constant k can be estimated according to the formula: ln Rt/R0=kt.

Relative amounts of each imported RNA inside mitochondria were estimated after quantification in the Typhoon-Trio scanner as a ratio between the signal obtained after hybridization with a specific probe and the signal obtained after hybridization with a probe against the mitochondrial tRNAThr in the same RNA preparation, as described previously.

Mutant mtDNA Heteroplasmy Level Analysis

Heteroplasmy level was measured by real-time PCR using SYBR Green (iCycler, MyiQ Real-Time Detection System, BioRad) as described previously. Two pairs of primers were used: (1) amplifying the 210 by fragment of 12S rRNA gene region (nucleotides 1095-1305 in mtDNA) not touched by the KSS deletion as a value showing all mtDNA molecules, and (2) amplifying the 164 by fragment of the deleted region (nucleotides 11,614-11,778) as a value showing wild-type mtDNA molecules. All reactions were performed in a 20 ml volume in triplicates. PCR using water instead of template was used as a negative control. PCR was performed by initial denaturation at 95° C. for 10 min, followed by 40 cycles of 30 s at 95° C., 30 s at 60° C., and 30 s at 72° C. Specificity was verified by melting curve analysis and gel electrophoresis.

Chemical Synthesis of Modified Oligonucleotides

All the oligonucleotides (FIG. 5) were synthesized on an automatic ASM-800 RNA/DNA synthesizer (Biosset, Novosibirsk, Russia) at 0.4 mmol scale using solid phase phosphoramidite synthesis protocols optimized for this instrument. The DNA phosphoramidites, 2′-O-TBDMS-protected RNA phosphoramidites, 2′-O-Me pyrimidine RNA phosphoramidites, 2′-F-pyrimidine RNA phosphoramidites and appropriate supports were purchased from Glen Research (USA). Attachment of 3′-O-DMTr-thymidine to the CPG-500 solid carrier (SigmaeAldrich) containing carboxylic groups was carried out as in the state of the art. A 5-ethylthio-H-tetrazole has been used as activator with 5, 10, 6 and 10 min coupling steps respectively for the phosphoramidites mentioned above. After standard deprotection, oligonucleotides were isolated via preparative electrophoresis in 12% polyacrylamide/8 M urea gel, followed by elution with 0.3 M NaOAc (pH 5.2)/0.1% SDS solution and precipitated with ethanol as Na+salts. Purified oligonucleotides were characterized by electrophoretic mobility in 12% denaturing polyacrylamide gel and by MALDI-TOF mass spectrometry (Autoflex III, Bruker Daltonics, Germany).

Synthetic RNA Hybridization and Thermal Denaturation Assays

To predict melting temperatures for DNA-DNA and RNA-DNA duplexes, we used IDT Sci-Tools OligoAnalyser 3.1 software. Thermal denaturation experiments were performed on a Cary 300 BioMelt Spectrophotometer equipped with Temperature Probes Accessory Series II (Varian Inc., Australia) in a buffer containing 10 mM sodium cacodylate, pH 7.4, 150 mM NaCl and 2 mM MgCl2. The oligonucleotides (2.0 mM each strand) were mixed, denatured by heating and subsequently cooled to the starting temperature of the experiment. The rate of temperature change was 0.5° C./min. Duplex melting temperatures (Tm) were calculated using the thermodynamic parameters DH and DS that have been obtained by fitting procedure of UV-melting curves registered at two different wavelengths (260 and 270 nm) according to the two-state model. The parameter determination error did not exceed 5-10%. Specific hybridization with target mtDNA was tested using Southern-blot hybridization of wild-type (nucleotides 15,251-15,680 of mtDNA) and KSS (nucleotides 8099-8365/15,438-15,680 of mutant mtDNA) fragments with 32P-labelled synthetic RNAs in 1×PBS at 37° C. as described previously.

Recombinant RNA Modelling and Synthesis

To predict secondary structures of recombinant RNA and estimate their free energy (dG), the Mfold (15), IDT Sci-Tools OligoAnalyser 3.1 and ViennaRNA platforms were used. To estimate melting temperatures for DNA-DNA and RNA-DNA duplexes, we used IDT Sci-Tools OligoAnalyser 3.1 software (16).

The recombinant RNAs were obtained by T7 transcription using the T7 RiboMAX Express Large Scale RNA Production System (Promega) on the corresponding PCR products and gel-purified. For PCR, the following oligonucleotides containing a T7 promoter (underlined) were used:

(SEQ ID NO: 44)
T7+FD16L (5′-TAA TAC GAC TCA CTA TAG CGC AAT CGG
TAG CGC ACT CCA AAG GCC ACA TGA GCC CCC TAC AGG
GCT C-3′);
(SEQ ID NO: 45)
T7+FD16H (5′-TAA TAC GAC TCA CTA TAG CGC AAT CGG
TAG CGC ATG TGG CCT TTG GAG TGA GCC CCC TAC AGG
GCT C-3′);
(SEQ ID NO: 46)
T7+FD20L (5′-TAA TAC GAC TCA CTA TAG CGC AAT CGG
TAG CGC ACT CCA AAG GCC ACA TCA TCG AGC CCC CTA
CAG GGC TC-3′);
(SEQ ID NO: 47)
T7+FD20H (5′-TAA TAC GAC TCA CTA TAG CGC AAT CGG
TAG CGC GAT GAT GTG GCC TTT GGA GTG AGC CCC CTA
CAG GGC TC-3′);
(SEQ ID NO: 48)
T7+FD25L (5′-TAA TAC GAC TCA CTA TAG CGC AAT CGG
TAG CGC GTT TCT ACT CCA AAG GCC ACA TCA TGA GCC
CCC TAC AGG GCT C-3′);
(SEQ ID NO: 49)
T7+FD25H (5′-TAA TAC GAC TCA CTA TAG CGC AAT CGG
TAG CGC ATG ATG TGG CCT TTG GAG TAG AAA CGA GCC
CCC TAC AGG GCT C-3′)
with primers T7
(SEQ ID NO: 50)
(5′-GGG ATC CAT AAT ACG ACT CAC TAT A-3′)
(SEQ ID NO: 51)
and FD-rev (5′-AAG AGC CCT GTA GGG-3′);
(SEQ ID NO: 52)
FD16L (5′-G CGC AAU CGG UAG CGC ACU CCA AAG GCC
ACA UGA GCC CCC UAC AGG GCU CUU-3′);
(SEQ ID NO: 53)
FD16H (5′-G CGC AAU CGG UAG CGC AUG UGG CCU UUG
GAG UGA GCC CCC UAC AGG GCU CUU-3′);
(SEQ ID NO: 54)
FD20L (5′-G CGC AAU CGG UAG CGC ACU CCA AAG GCC
ACA UCA UCG AGC CCC CUA CAG GGC UCU U-3′);
(SEQ ID NO: 55)
FD20H (5′-G CGC AAU CGG UAG CGC GAU GAU GUG GCC
UUU GGA GUG AGC CCC CUA CAG GGC UCUU-3′);
(SEQ ID NO: 56)
FD25L (5′-G CGC AAU CGG UAG CGC GUU UCU ACU CCA
AAG GCC ACA UCA UGA GCC CCC UAC AGG GCU CUU-3′);
(SEQ ID NO: 57)
FD25H (5′-G CGC AAU CGG UAG CGC AUG AUG UGG CCU
UUG GAG UAG AAA CGA GCC CCC UAC AGG GCU CUU-3′).

To test the recombinant RNA annealing to target mtDNA, fragments of wild-type and mutant mtDNA were amplified using primers hmtGluRT (5′-GTT CTT GTA GTT GAA ATA C-3′) (SEQ ID NO: 58) and CRC-F (5′-CAT ACC TCT CAC TTC AAC CTC C-3′) (SEQ ID NO: 59), separated on 1% agarose gel, blotted to Hybond-N membrane and hybridized with 32P-labelled recombinant RNAs in 1×PBS at 37° C. After PhosphorImager quantification (Typhoon-Trio, GE Healthcare), the hybridisation signals were normalized to amounts of corresponding mtDNA fragments calculated after ethidium bromide staining of the agarose gel (before the transfer to Hybond membrane) by densitometry using G-Box and GeneTools analysis software. Thereafter, hybridization specificity was calculated for each RNA as 1−(ratio between normalized signals for wild-type and mutated DNA fragments). Thus, for recombinant RNA annealed only to mutant DNA fragment, the hybridization specificity value reached 1, and for RNA annealed equally to mutant and wild-type fragments, this value was close to 0. At least three independent experiments were performed for each RNA.

Chimeric oligonucleotide synthesis—Chimeric oligonucleotides D20L-DNA and D20H-DNA were synthesized on an automatic ASM-800 RNA/DNA synthesizer (Biosset, Russia) at 0.4 μmol scale using solid phase phosphoramidite synthesis protocols (17) optimized for this instrument. The DNA and 2′-O-TBDMS-protected RNA phosphoramidites and appropriate supports were purchased from Glen Research (USA). A 5-ethylthio-H-tetrazole has been used as activator with 5 and 10 min coupling steps, respectively. After standard deprotection, oligonucleotides were purified by 12% polyacrylamide/8M urea gel and characterized by MALDI-TOF mass spectrometry (Autoflex III, Bruker Daltonics).

Synthesis of fluorescently labeled RNA transcript—Alexa Fluor 488-5 UTP (Molecular Probes) was incorporated into RNA during 2 h T7-transcription by T7-RNA polymerase (Promega). Reaction mixture of total volume 20 μl contained 0.5 μg of DNA template, 80 u of T7-RNA polymerase (2 additions of 40 u each), 0.5 mM of ATP, 0.5 mM CTP, 0.5 mM GTP, 0.37 mM UTP, 0.125 mM Alexa Fluor 488-5-UTP, 10 mM DTT and 40 u of RNaseOUT (Invitrogen). T7-transcript was purified by PAGE. To check the incorporation of the label in purified transcript, the dye absorbance at 492 nm was compared with the absorbance at 260 nm using NanoDrop ND1000 V3.5.2 Microarray software. The efficiency of labelling obtained was approximately two labeled uridines per one RNA molecule.

RNA FD20H, used for the microscopy, contains 13 uridine residues (FIG. 2): one at the end of F-stem region, three in the loops and nine in the anti-replicative part of the molecule, which is not responsible for the mitochondrial import efficiency. Therefore, the labelling should not significantly alter the secondary structure and mitochondrial import of the RNA molecule.

Transient transfection of cybrid cells—Transmitochondrial cybrid cell lines obtained by fusion of fibroblast-derived cytoplast from patient and a 143B rho° cell line (18) were kindly provided by M. Zeviani (National Neurological Institute ‘Carlo Besta’ of Milan).

Primary skin fibroblasts were from Patient 1-a boy born to non-consanguineous healthy parents. He was completely normal until 13 years of age when he developed dystonia of the left hand that progressively worsened. Brain MRI at 14 years of age revealed hypersignal of the basal ganglia and abnormalities of brain system consistent with the diagnosis of Leigh syndrome. Two years after he had tonicoclonic seizures with nystagmus and dysarthria. Stroke-like was detected by brain MRI. Measurement of respiratory chain activities detected an isolated complex I deficiency related to a mitochondrial DNA mutation in ND5 gene (13514A>G). Fibroblasts were characterised by 30% heteroplasmy level. Cells were cultivated at 37° C. and 5% CO2 in DMEM (Sigma) containing 4.5 g/L of glucose supplemented with fetal calf serum (Gibco), penicillin/streptomycin, uridine (50 mg/L) and fungizone (2.50 mg/L) (Gibco).

Transient transfection of cells with RNA and chimeric molecules was performed as described in (19,20) with some modifications: for 2 cm2 well of 80% confluent cells, 0.25 μg RNA was used and 1 μl of Lipofectamine 2000 in OptiMEM medium (Invitrogen). OptiMEM was changed to a standard DMEM medium 8 hours after the transfection. In these conditions, about 80% of fibroblasts and 95% of cybrid cells were transfected with RNA (evaluated by fluorescent microscopy and by flow cytometry using CyFlow FACS 24 h post transfection). By Northern hybridisation of total cellular RNA comparing to the signals of the known amounts of T7-transcript loaded on the same gel, the inventors could estimate that about 15% of RNA added to cybrid cells, and 20% RNA for fibroblasts, were internalised and can be detected in full-size form 24 h post transfection (not shown). The transfection procedure did not lead to a detectable decrease in viability of the cells or to a significant change of the overall mtDNA amount verified by real-time qPCR as described (14).

RNA stability and mitochondrial import in vivo—Mitochondria were isolated from the cybrid cells as described earlier (21,22), treated with digitonin to generate mitoplasts (mitochondria devoid of the outer membrane) and RNase A to get rid of non-specifically attached RNA. Such a treatment allows to obtain mitochondria free of cytosolic RNA contamination, which was tested using a probe for cytosolic 5.8S rRNA, normally strongly attached to the outer mitochondrial membrane upon cell disruption. Total and mitochondrial RNA were isolated with TRIzol reagent (Invitrogen).

Stability and mitochondrial import of recombinant molecules were analyzed by Northern hybridization of total and mitochondrial RNA with 32P-labelled oligonucleotide probes followed by PhosphorImager quantification (Typhoon-Trio, GE Healthcare).

The probes used: “D-loop” probe specific for recombinant molecules (see FIG. 1), (5′-GAG TCA TAC GCG CTA CCG ATT GCG CCA ACA AGG C-3′) (SEQ ID NO: 39), hybridization temperature 50° C.; cytosolic 5.8S rRNA probe, (5′-GGC CGC AAG TGC GTT CGA AG-3′) (SEQ ID NO: 40), hybridization temperature 50° C.; probe against the mitochondrial tRNAVa1, (5′-GAA CCT CTG ACT GTA AAG-3′) (SEQ ID NO: 60), hybridization temperature 45° C.; probe against the nuclear snRNA U3, (5′-CGC TAC CTC TCT TCC TCG TGG-3′) (SEQ ID NO: 61), hybridization temperature 50° C.

To compare the stability of different recombinant RNA, the relative concentration of each RNA in various time periods after transfection was calculated as a ratio between the D-loop probe signal and the signal for cytosolic 5.8S rRNA.

The relative amount of each imported RNA inside the mitochondria was estimated as a ratio between the signal obtained after hybridization with the D-loop probe and the signal with a probe against the mitochondrial tRNAVa1 in the same RNA preparation. To compare import efficiencies of different RNA molecules, the total level of each RNA molecule in transfected cells was taken into account and normalized as described previously (21). At least three independent experiments were performed for each RNA.

Confocal microscopy—Cybrid cells cultivated in 2 cm2 chambers slide (Lab-Tek) were transfected with Alexa Fluor 488-5-UTP labelled RNA. At different time periods after transfection, living cells were stained with TMRM (Tetramethylrhodamine Methyl Ester), 5 μM for 15 min at 37° C., washed and imaged in DMEM without red phenol. The LSM 700 confocal microscope (Zeiss) was used in conjunction with Zen imaging software and images acquired with a Zeiss 63×/1.40 oil immersion objective with resolution 1024×1024. The excitation/emission laser wavelengths were 488 nm (green channel) and 555 nm (red channel). Images were analysed using ImageJ (23) and JACoP plugin (24). Co-localization analysis were performed on multiple cells and optical sections. Pearson's and Manders' coefficients were estimated (25,26). Pearson's correlation coefficient provides a measure of correlation between the intensities of each channel in each pixel. The overlap coefficients according to Manders indicate an actual overlap of the signals and represent the true degree of co-localization.

In control experiments, cells were transfected with Alexa Fluor 488-5-UTP labelled RNA, which is not imported into mitochondria (27).

MtDNA heteroplasmy level analysis—To isolate total DNA from transfected cells, 1 cm2 of cells were solubilized in 0.5 ml of buffer containing 10 mM Tris-HCl, pH 7.5, 10 mM NaCl, 25 mM Na-EDTA and 1% SDS, then 10 μl of proteinase K solution (20 mg/ml) was added and the mixture incubated for 2 h at 50° C.; 50 μl of 5M NaCl was added, the DNA was precipitated with isopropanol and used for PCR amplification. Heteroplasmy level was analysed by restriction fragment length polymorphism (RFLP) on a 125-bp PCR fragment encompassing nucleotides 13430 to 13555 of mtDNA obtained with primers CRC-F 5′-CAT ACC TCT CAC TTC AAC CTC C-3′ (SEQ ID NO: 62) and CRC-R 5′-AGG CGT TTG TGT ATG ATA TGT TTG C-3′ (SEQ ID NO: 63) (18). The A13514G mutation creates an HaeIII-specific cleavage site, giving two fragments of 80 and 45 bp. The HaeIII-digested fragments were separated on a 10% PAGE and stained with ethidium bromide. The proportion of mutant versus total mtDNA was calculated by densitometry using G-Box and GeneTools analysis software (Syngene). At least four independent transfections were performed with each recombinant RNA. Each DNA sample was analysed twice by PCR amplification followed by at least three gel separation and quantification experiments.

The digestion control was performed for each reaction. For this, a 600-bp PCR fragment encompassing nucleotides 1216 to 1813 of mtDNA obtained with primers CRS-F 5′-CGA TAA ACC CCG ATC AAC CTC-3′ (SEQ ID NO: 64) and CRS-R 5′-GGT TAT AAT TTT TCA TCT TTC CC-3′ (SEQ ID NO: 65) was cleaved by HaeIII to 350 and 250-bp fragments in the same tube as a 125-bp PCR fragment containing the A13514G mutation site. Mitochondria quantification—2 cm2 wells of 80% confluent cybrid cells and control wild-type 143B cells, transfected with FD20H RNA, were stained with 5 μM TMRM and 200 nM MitoTracker® Green during 15 min in culture media. The intensity of fluorescence was directly measured in a culture plate in DMEM without red phenol by VICTOR™ ×3 multilabel plate reader (Perkin-Elmer) at 485 nm (green channel) and 555 nm (red channel), in triplicate for each sample. After fluorescence measurement, the cells were detached and quantified by flow cytometry using CyFlow FACS and FloMax software (Partec). Activity of complex I was measured using a kit MitoSciences® (Abcam).

Statistical analyses—Pairwise comparisons were performed using two-tailed Student's t-test, Excel software (Microsoft). Data is expressed as mean±s.d.

### REFERENCES

1. Sepuri, N. B., et al. (2012) PLoS One 7, e35321
2. Ruiz-Pesini, E., et al. (2007) Nucleic Acids Res 35, D823-828
3. Wallace, D. C. (2010) Environmental and molecular mutagenesis 51, 440-450
4. Rustin, P., H, T. J., et al. (2007) Med Sci (Paris) 23, 519-525
5. Manfredi, G., et al. (2002) Nat Genet 30, 394-399
6. Schneider, A. (2011) Annu Rev Biochem 80, 1033-1053
7. Karicheva, 0. Z., et al. (2011) Nucleic Acids Res 39, 8173-8186
8. Kolesnikova, 0. A., et al. (2004) Hum Mol Genet 13, 2519-2534
9. Bacman, S. R., et al. (2010) Gene Ther 17, 713-720
10. Bacman, S. R., et al. (2007) Gene Ther 14, 1309-1318
11. Taylor, R. W., et al. (1997) Nat Genet 15, 212-215
12. Muratovska, A., et al. (2001) Nucleic Acids Res 29, 1852-1863.
13. Kolesnikova, O., et al. (2010) RNA 16, 926-941
14. Comte, C., T et al. (2013) Nucleic Acids Res 41, 418-433
15. Zuker, M. (2003) Nucleic Acids Res 31, 3406-3415
16. Sugimoto, N., et al. (1995) Biochemistry 34, 11211-11216
17. Bellon, L. (2001) Curr Protoc Nucleic Acid Chem, Chapter 3, Unit 3 6
18. Corona, P., et al. (2001) Ann Neurol 49, 106-110
19. Smirnov, A. V., et al. (2008) Biochemistry (Mosc) 73, 1418-1437
20. Kohrer, C., et al. (2001) I Proc Natl Acad Sci USA 98, 14310-14315
21. Smirnov, A., et al. (2008) T RNA 14, 749-759
22. Mager-Heckel, A. M., et al. (2007) Methods Mol Biol 372, 235-253
23. Schneider, C. A., et al. (2012) Nat Methods 9, 671-675
24. Bolte, S., and Cordelieres, F. P. (2006) J Microsc 224, 213-232
25. Zinchuk, V., et al. (2007) Acta Histochem Cytochem 40, 101-111
26. Zinchuk, V., and Grossenbacher-Zinchuk, O. (2009) Prog Histochem Cytochem 44, 125-172
27. Gowher, A., et al. (2013) PLoS One 8, e66228
28. Tonin, Y., et al. (2014) Biochimie 100, 195-199
29. Blok, M. J., et al. (2007) J Med Genet 44, e74
30. Davidson, S. M., et al. (2007) Methods Mol Biol 372, 421-430
31. Efremov, R. G., and Sazanov, L. A. (2012) Biochim Biophys Acta 1817, 1785-1795
32. Ohnishi, T., O et al. (2012) Biochim Biophys Acta 1817, 1803-1809
33. Baradaran, R., et al. (2013) Nature 494, 443-448
34. Efremov, R. G., and Sazanov, L. A. (2011) Nature 476, 414-420
35. Brautbar, A., et al. (2008) Molecular genetics and metabolism 94, 485-490
36. Bugiani, M., J et al. (2004) Biochim Biophys Acta 1659, 136-147
37. Chol, M., L et al. (2003) J Med Genet 40, 188-191
38. Sudo, A., et al. (2004) J Hum Genet 49, 92-96
39. Diaz, F., et al. (2002) Nucleic Acids Res 30, 4626-4633
40. Bayona-Bafaluy, M. P., et al. (2005) Proc Natl Acad Sci USA 102, 14392-14397
41. Suen, D. F., et al. (2010) Proc Natl Acad Sci USA 107, 11835-11840
42. Bowmaker, M., et al. (2003) J Biol Chem 278, 50961-50969
43. Presley, A. D., et al. (2003) J Chromatogr B Analyt Technol Biomed Life Sci 793, 141-150

1. A mitochondrial vector comprising or consisting essentially of a first nucleic acid sequence linked to a second nucleic acid sequence,
wherein the first nucleic acid sequence is the sequence of D-arm as disclosed in SEQ ID NO: 1 or a sequence having at least 75% sequence identity with SEQ ID NO: 1 or the sequence of F-hairpin as disclosed in SEQ ID NO: 5 or a sequence having at least 75% sequence identity with SEQ ID NO: 5;
wherein the second nucleic acid sequence is a sequence of interest; and
wherein the mitochondrial vector does not comprise both SEQ ID NOs: 1 and 2 or sequences having at least 75% sequence identity with SEQ ID NOs: 1 and 5.

2. The mitochondrial vector according to claim 1, wherein the first nucleic acid sequence is a sequence having at least 90% sequence identity with SEQ ID NOs: 1 or 5.

3. The mitochondrial vector according to claim 1, wherein the first nucleic acid sequence is a sequence having at least 95% sequence identity with SEQ ID NOs: 1 or 5.

4. The mitochondrial vector according to claim 1, wherein the second nucleic acid sequence is a wild-type mitochondrial DNA sequence or an altered mitochondrial DNA sequence or a complementary sequence thereof.

5. The mitochondrial vector according to claim 1, wherein the second nucleic acid sequence is an altered mitochondrial DNA sequence or a complementary sequence thereof.

6. The mitochondrial vector according to claim 1, wherein the second nucleic acid sequence of interest is between 10 to 30 nucleotides in length, preferably between 15 to 25 nucleotides in length.

7. The mitochondrial vector according to claim 1, wherein the mitochondrial vector is between 26 to 48 nucleotides in length.

8. The mitochondrial vector according to claim 1, wherein the sequence of D-arm as disclosed in SEQ ID NO: 1 or a sequence having at least 75% sequence identity with SEQ ID NO: 1 is linked at the 5′ end of the second nucleic acid sequence.

9. The mitochondrial vector according to claim 8, wherein the sequence of D-arm as disclosed in SEQ ID NO: 1 or a sequence having at least 75% sequence identity with SEQ ID NO: 1 is at the 5′ end of the mitochondrial vector.

10. The mitochondrial vector according to claim 1, wherein the sequence of F-hairpin as disclosed in SEQ ID NO: 5 or a sequence having at least 75% sequence identity with SEQ ID NO: 5 is linked at the 3′ end of the second nucleic acid sequence.

11. The mitochondrial vector according to claim 10, wherein the sequence of F-hairpin as disclosed in SEQ ID NO: 5 or a sequence having at least 75% sequence identity with SEQ ID NO: 5 is at the 3′ end of the mitochondrial vector or is located before the 1-5 nucleotides of the 3′ end of the mitochondrial vector.

12. The mitochondrial vector according to claim 1, wherein the first nucleic acid sequence is made of ribonucleotides.

13. The mitochondrial vector according to claim 1, wherein the second nucleic acid sequence comprises deoxyribonucleotides, ribonucleotides or a mixture thereof, and/or 2′-O-methylated ribonucleotide(s) and/or inverted 3′-3′ or 5′-5′ nucleotides.

14. The mitochondrial vector according to claim 1, wherein the mitochondrial vector comprises or essentially consists of a sequence selected from SEQ ID NOs: 2-4.

15. The mitochondrial vector according to claim 1, wherein the mitochondrial vector is conjugated with a delivery system such as a cholesterol.

16. A pharmaceutical composition comprising a mitochondrial vector according to claim 1 and a pharmaceutically acceptable carrier or excipient.

17. A method of treating in a subject a mitochondrial disease caused by a mutation in a gene in the mitochondrial genome which comprises administering to the subject the mitochondrial vector of claim 1, wherein the second nucleic acid sequence is a sequence comprising the mutation of the gene or its complementary sequence.

18. The method of claim 17, wherein the mutation in a gene in the mitochondrial genome is selected from the group consisting of: tRNAleu-A3243G, A3251 G, A3303G, T3250C, T3271 C and T3394C; tRNAlys-A8344G, G11778A, G8363A, and T8356C; ND1-G3460A; ND4-A10750G and G14459A; ND6-T14484A; 12S rRNA-A1555G; MTTS2-C12258A; ATPase 6-T8993G, and T8993C; tRNASer(UCN)-T7511C; 11778 and 14484, LHON mutations and mutations or deletions in ND2, ND3, NDS, cytochrome b, cytochrome oxidase MIL and ATPase 8.

19. A kit comprising a mitochondrial vector according to claim 1.
